(19) United States
(12) Patent Application Publication
(10) Pub. No.: US 2010/0323348 A1
(43) Pub. Date: Dec. 23, 2010

(54) Methods and Compositions for Using Error-Detecting and/or Error-Correcting Barcodes in Nucleic Acid Amplification Process

(75) Inventors: Micah L. Hamady, Berkeley, CA (US); Robin D. Knight, Lafayette, CO (US)

Correspondence Address: MEDLEN & CARROLL, LLP, 101 HOWARD STREET, SUITE 350, SAN FRANCISCO, CA, 94105, US

(73) Assignee: The Regents of the University of Colorado, a body corporate, Denver, CO (US)

(21) Appl. No.: 12/693,612

(22) Filed: Jan. 26, 2010

Related U.S. Application Data
(60) Provisional application No. 61/148,931, filed on Jan. 31, 2009.

(51) Int. Cl. C12Q 1/68; C07H 21/00

(52) U.S. Cl. 435/6; 536/24.33

(57) ABSTRACT

The present invention provides methods and compositions for detecting and correcting errors in nucleic acid amplification processes, and methods for using the same. In particular, barcode amplification errors are detected and corrected such that integrity in sample assignment is maintained. The methods are compatible with high throughput sequencing techniques as some of the barcodes are based upon Hamming codes, thereby allowing self-correction for single bit errors. Some methods and compositions of the invention allow characterization (e.g., sequencing) of a plurality of nucleic acid samples simultaneously within a single sequencing reaction.

### STATEMENT REGARDING FEDERALLY FUNDED RESEARCH This invention was made with government support under Grant Nos. T32GM065103 and P01DK078669 awarded by the National Institutes of Health. The government has certain rights in the invention.

### FIELD OF THE INVENTION

The present invention relates to nucleic acid sequencing. In particular, the invention relates to methods and compositions for detecting errors and correcting such errors during nucleic acid amplification such that accurate sample identification may be maintained. The combination of the methods and compositions described herein allow characterization of a plurality of nucleic acid samples simultaneously when using high throughput amplification and/or sequencing technologies.

### BACKGROUND OF THE INVENTION

DNA barcodes were first developed as a tool for species-level identifications. Consequently, there is a rapidly growing database of these short sequences from a wide variety of taxa. Correlations have also been drawn between the nucleotide content of the short DNA barcode sequences and the genomes from which they are derived. Consequently, short nucleotide sequences can reliably track information about the composition of the entire genome. Min et al.,. “DNA barcodes provide a quick preview of mitochondrial genome composition” PLoS One 2(3):e325 (2007).

In the past several years, microarray technologies based on whole genome analysis have been applied to the study of gene expression and/or amplification. Microarrays arose out of the development of large-scale sequencing approaches and generate a far greater volume of data than the data representing the sequences themselves. Ghosh D., “High throughput and global approaches to gene expression” Comb Chem High Throughput Screen 3:411-20 (2000). The current state of development of microarray expression and/or amplification has overshadowed conventional sequencing methods and the associated approaches to manage and analyze the information they generate.

What is needed in the art is an efficient, low cost method for tracking and identifying specific nucleic acids during polymerase chain reaction amplification that is compatible with conventional high throughput data generation technology.

### SUMMARY OF THE INVENTION

The present invention relates to nucleic acid sequencing. In particular, the invention relates to methods and compositions for detecting errors and correcting such errors during nucleic acid amplification such that accurate sample identification may be maintained. The combination of the methods and compositions described herein allow characterization of a plurality of nucleic acid samples simultaneously when using high throughput amplification and/or sequencing technologies.

In one embodiment, the present invention contemplates methods and compositions comprising primers encoding error-correcting sequence tags and/or error-detecting sequence tags (i.e., for example, error-correcting barcodes and/or error-detecting barcodes).

In one embodiment, the present invention contemplates a pyrosequencing compatible primer comprising a first region containing a unique error-detecting/correcting hamming barcode. In one embodiment, the primer further comprises a second region complementary to a bacterial 16S rRNA gene. In one embodiment, the barcode is attached to the 3′ end of the primer. In one embodiment, the barcode is attached to the 5′ end of the primer. In one embodiment, the barcode is attached to the 3′ end and the 5′ end of the primer.

In one embodiment, the present invention contemplates a method of assigning sequence data to individual samples from a mixture of samples, comprising: a) providing: i) a pyrosequencing compatible primer comprising a first region containing a unique error-detecting/correcting barcode and a second region complementary to a target nucleic acid molecule and, and ii) a target nucleic acid molecule, b) amplifying said target nucleic acid molecule with said primer, c) pooling a plurality of said amplification product, and d) pyrosequencing said pooled amplification products to determine their respective nucleotide sequences. In one embodiment, the plurality of amplification products are pooled in equimolar ratios. In one embodiment, the unique error-detecting/correcting barcode is a Hamming code. In one embodiment, the target nucleic acid molecule comprises a portion of the 16S rRNA gene. In one embodiment, the barcode is attached to the 3′ end of the primer. In one embodiment, the barcode is attached to the 5′ end of the primer. In one embodiment, the barcode is attached to the 3′ end and the 5′ end of the primer. In one embodiment, the method further comprises identifying amplification products with unique barcode sequence errors. In one embodiment, the compositions are used in parallel sequencing runs, wherein a plurality of sequencing assays are performed simultaneously. In one embodiment, the sequencing assay comprises pyrosequencing wherein nucleic acid sequences from many samples may be characterized simultaneously in a nucleic acid amplification process. In one embodiment, the method further comprising correcting the unique barcode sequence of amplification products containing correctable unique barcode sequence errors. In one embodiment, the method further comprises discarding the nucleotide sequence of amplification products containing non-correctable unique barcode sequence errors. In one embodiment, the method further comprises aligning the nucleotide sequences of said amplification products to generate a phylogenetic tree.

In one embodiment, the present invention contemplates a method comprising: a) providing: i) a plurality of samples comprising nucleic acid sequences; i) a plurality of primers error correcting and/or error-detecting sequence tags (i.e., for example, ‘barcodes’), wherein said primers are at least partially complementary to said nucleic acid sequences: ii) a parallel sequencing technique (i.e., for example, pyrosequencing) capable of simultaneously characterizing said nucleic acid sequences from said plurality of samples; b) amplifying said plurality of nucleic acid samples using said plurality of primers; and c) analyzing said sequence tags of said amplified nucleic acids. In one embodiment, the sequence tag identifies a sample assignment thereby identifying one of said samples from which said nucleic acid was derived. In one embodiment, the sequence tag identifies the presence of an error in said nucleic acid, thereby establishing a probability that said sample assignment is incorrect. In one embodiment, the sequence tag identifies the absence of any error in said nucleic acid, thereby establishing a probability that said sample assignment is correct.

### DEFINITIONS

The term “parity bit” as used herein, refers to any bit that is added to a bit-coded string (i.e., for example, a series of “ones” and zeros”) to ensure that the number of bits with the value one in a set of bits is even or odd. Parity bits are used as the simplest form of error detecting code. For example, two variants of parity bits may include, but are not limited to, an even parity bit and an odd parity bit. When using even parity, the parity bit is set to 1 if the number of ones in a given set of bits (not including the parity bit) is odd, making the entire set of bits (including the parity bit) even. When using odd parity, the parity bit is set to 1 if the number of ones in a given set of bits (not including the parity bit) is even, making the entire set of bits (including the parity bit) odd. In other words, an even parity bit will be set to “1” if the number of 1's+1 is even, and an odd parity bit will be set to “1” if the number of 1's+1 is odd.

The term “parallel sequencing technique” as used herein, refers to any method capable of sequencing multiple templates at one time (i.e., for example, simultaneously). Usually, such techniques are performed by immobilizing either a template or primer on a solid support (i.e., for example, a microarray) configured to support a high throughput process. Pyrosequencing is compatible with most parallel, or massively parallel, sequencing technologies. Fuller C. W., “Rapid parallel nucleic acid analysis” U.S. Pat. No. 7,264,934 (herein incorporated by reference).

The term “pyrosequencing” as used herein, refers to any pyrophosphate-based nucleic acid sequencing method. Hyman U.S. Pat. No. 4,971,903 (herein incorporated by reference). This technique is based on the observation that pyrophosphate (PPi) is released upon incorporation of the next correct nucleotide 3′ of the primer sequence. For example, when only one of the four nucleotides (i.e., for example, A, T, G, C) is introduced into the reaction at a time, PPi is generated only when the correct nucleotide is introduced. Thus, the production of PPi reveals the identity of the next correct base. Using this process in an iterative fashion results in the identification of the template nucleotide sequence. Pyrosequencing is compatible with most high throughput sequencing techniques, such as using template carrying microbeads deposited in microfabricated picoliter-sized reaction wells. Margulies et al., Nature E-Pub 31 Jul. 2005.

The term “simultaneously” as used herein refers to any two or more processes that are occurring more or less at the same time. It is not intended that each process begin and end precisely together, but only that their respective durations overlap.

The term “pyrosequencing compatible primer” as used herein, refers to any primer, or primer pair, that is capable of supporting nucleic acid amplification using any pyrosequencing technology.

The term “unique error-detecting/correcting Hamming barcode” or “Hamming sequence tag” as used herein, refers to any nucleic acid barcode having a unique sequence identified by the concepts and algorithms associated with Hamming codes (infra).

The term “Hamming code” as used herein, refers an arithmetic process that identifies unique binary codes based upon inherent redundancy that are capable of correcting single bit errors. For example, a Hamming code can be matched with a nucleic acid barcode in order to screen for single nucleotide errors occurring during nucleic acid amplification. The identification of a single nucleotide error by using a Hamming code, thereby allows for the correction of the nucleic acid barcode.

The term “sample assignment” as used herein, refers to any established relationship between the source of a specific nucleotide and an attached barcode. For example, when a unique barcode is cross-referenced with a specific geographic location as to where the nucleotide was obtained, the nucleotide has a sample assignment of that specific geographic location.

The term “equimolar ratios” as used herein, refers to any mixture comprising at least two components, wherein the concentration of each component is the same.

The term “amplification products” as used herein, refers to any nucleotide produced by the replication and/or amplification of DNA or RNA. For example, mRNA may be amplified into cDNA by reverse transcriptase. Alternative, a DNA template may undergo amplification of at least one of its strands during a polymerase chain reaction (PCR) thereby producing amplification products whose composition is dependent upon the primer pair.

The term “unique barcode sequence error” as used herein, refers to any alteration in a barcode nucleic acid sequence occurring during amplification.

The term “correctable unique barcode sequence error” as used herein, refers to any single bit error occurring in a barcode nucleic acid sequence during amplification.

The term “uncorrectable unique barcode sequence error” as used herein, refers to any bit error that is greater than an single bit error (i.e., for example, a two bit, three bit, four bit etc) error occurring during amplification.

The term “discarding” as used herein, refers to any process that does not rely on a barcode nucleic acid sequence comprising an uncorrectable unique barcode sequence error. Such an error results in an improper sample assignment for the coded nucleic acid thereby resulting in a mis-classification.

The term “phylogenetic tree” as used herein, refers to any diagram or other similar representation showing the evolutionary relationships among various biological species or other entities that are known to have a common ancestor. For example, a phylogenetic tree may comprise nodes with descendants representing the most recent common ancestor of the descendants, and the edge lengths in some trees may correspond to time estimates.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). For example, a pulmonary sample may be collected by bronchoalveolar lavage (BAL) which comprises fluid and cells derived from lung tissues. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “derived from” as used herein, refers to the source of a compound or sequence. In one respect, a compound or sequence may be derived from an organism or particular species. In another respect, a compound or sequence may be derived from a larger complex or sequence. “Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

As used herein the term “portion” or “region” when in reference to a protein (as in “a portion or region of a given protein”) refers to fragments of that protein. The fragments may range in size from four amino acid residues to the entire amino acid sequence minus one amino acid.

The term “portion” or “region” when used in reference to a nucleotide sequence refers to fragments of that nucleotide sequence. The fragments may range in size from 5 nucleotide residues to the entire nucleotide sequence minus one nucleic acid residue.

The term “functionally equivalent codon”, as used herein, refers to different codons that encode the same amino acid. This phenomenon is often referred to as “degeneracy” of the genetic code. For example, six different codons encode the amino acid arginine.

A “variant” of a protein is defined as an amino acid sequence which differs by one or more amino acids from a polypeptide sequence or any homolog of the polypeptide sequence. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Similar minor variations may also include amino acid deletions or insertions (i.e., additions), or both. Guidance in determining which and how many amino acid residues may be substituted, inserted or deleted without abolishing biological or immunological activity may be found using computer programs including, but not limited to, DNAStar® software.

A “variant” of a nucleotide is defined as a novel nucleotide sequence which differs from a reference oligonucleotide by having deletions, insertions and substitutions. These may be detected using a variety of methods (e.g., sequencing, hybridization assays etc.).

A “deletion” is defined as a change in either nucleotide or amino acid sequence in which one or more nucleotides or amino acid residues, respectively, are absent.

An “insertion” or “addition” is that change in a nucleotide or amino acid sequence which has resulted in the addition of one or more nucleotides or amino acid residues.

A “substitution” results from the replacement of one or more nucleotides or amino acids by different nucleotides or amino acids, respectively.

The term “derivative” as used herein, refers to any chemical modification of a nucleic acid or an amino acid. Illustrative of such modifications would be replacement of hydrogen by an alkyl, acyl, or amino group. For example, a nucleic acid derivative would encode a polypeptide which retains essential biological characteristics.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which is a “homolog” is defined herein as an oligonucleotide sequence which exhibits greater than or equal to 50% identity to a sequence, when sequences having a length of 100 bp or larger are compared.

Low stringency conditions comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent {50×Denhardt's contains per 500 ml: 5 g Ficoll (Type 400, Pharmacia), 5 g BSA (Fraction V; Sigma)} and 100 μg/ml denatured salmon sperm DNA followed by washing in a solution comprising 5×SSPE, 0.1% SDS at 42° C. when a probe of about 500 nucleotides in length. is employed. Numerous equivalent conditions may also be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) may also be used.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” is used in reference to the “melting temperature.” The melting temperature is the temperature at which a population of double-stranded nucleic acid molecules becomes half dissociated into single strands. As indicated by standard references, a simple estimate of the Tₘ value may be calculated by the equation: Tₘ=81.5+0.41 (% G+C), when a nucleic acid is in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tₘ.

As used herein, the term “stringency” is used in reference to the conditions of temperature, ionic strength, and the presence of other compounds such as organic solvents, under which nucleic acid hybridizations are conducted. “Stringency” typically occurs in a range from about Tₘ to about 20° C. to 25° C. below Tₘ. A “stringent hybridization” can be used to identify or detect identical polynucleotide sequences or to identify or detect similar or related polynucleotide sequences. For example, when fragments are employed in hybridization reactions under stringent conditions the hybridization of fragments which contain unique sequences (i.e., regions which are either non-homologous to or which contain less than about 50% homology or complementarity are favored. Alternatively, when conditions of “weak” or “low” stringency are used hybridization may occur with nucleic acids that are derived from organisms that are genetically diverse (i.e., for example, the frequency of complementary sequences is usually low between such organisms).

As used herein, the term “amplifiable nucleic acid” is used in reference to nucleic acids which may be amplified by any amplification method. It is contemplated that “amplifiable nucleic acid” will usually comprise “sample template.”

As used herein, the term “sample template” refers to nucleic acid originating from a sample which is analyzed for the presence of a target sequence of interest. In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

“Amplification” is defined as the production of additional copies of a nucleic acid sequence and is generally carried out using polymerase chain reaction. Dieffenbach C. W. and G. S. Dveksler (1995) In: PCR Primer, a Laboratory Manual, Cold Spring Harbor Press, Plainview, N.Y.

As used herein, the term “polymerase chain reaction” (“PCR”) refers to the method of K. B. Mullis U.S. Pat. Nos. 4,683,195 and 4,683,202, herein incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxy-ribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers; to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “restriction endonucleases” and “restriction enzymes” refer to bacterial enzymes, each of which cut double-stranded DNA at or near a specific nucleotide sequence.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription. Maniatis, T. et al., Science 236:1237 (1987). Promoter and enhancer elements have been isolated from a variety of eukaryotic sources including genes in plant, yeast, insect and mammalian cells and viruses (analogous control elements, i.e., promoters, are also found in prokaryotes). The selection of a particular promoter and enhancer depends on what cell type is to be used to express the protein of interest.

The presence of “splicing signals” on an expression vector often results in higher levels of expression of the recombinant transcript. Splicing signals mediate the removal of introns from the primary RNA transcript and consist of a splice donor and acceptor site. Sambrook, J. et al., In: Molecular Cloning: A Laboratory Manual, 2nd ed., Cold Spring Harbor laboratory Press, New York (1989) pp. 16.7-16.8. A commonly used splice donor and acceptor site is the splice junction from the 16S RNA of SV40.

The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is isolated from one gene and placed 3′ of another gene. Efficient expression of recombinant DNA sequences in eukaryotic cells involves expression of signals directing the efficient termination and polyadenylation of the resulting transcript. Transcription termination signals are generally found downstream of the polyadenylation signal and are a few hundred nucleotides in length.

The term “transfection” or “transfected” refers to the introduction of foreign DNA into a cell.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “Southern blot” refers to the analysis of DNA on agarose or acrylamide gels to fractionate the DNA according to size, followed by transfer and immobilization of the DNA from the gel to a solid support, such as nitrocellulose or a nylon membrane. The immobilized DNA is then probed with a labeled oligodeoxyribonucleotide probe or DNA probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists. J. Sambrook et al. (1989) In: Molecular Cloning: A Laboratory Manual, Cold Spring Harbor Press, NY, pp 9.31-9.58.

The term “Northern blot” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled oligodeoxyribonucleotide probe or DNA probe to detect RNA species complementary to the probe used. Northern blots are a standard tool of molecular biologists. J. Sambrook, J. et al. (1989) supra, pp 7.39-7.52.

The term “reverse Northern blot” as used herein refers to the analysis of DNA by electrophoresis of DNA on agarose gels to fractionate the DNA on the basis of size followed by transfer of the fractionated DNA from the gel to a solid support, such as nitrocellulose or a nylon membrane. The immobilized DNA is then probed with a labeled oligoribonuclotide probe or RNA probe to detect DNA species complementary to the ribo probe used.

As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

As used herein, the term “structural gene” refers to a DNA sequence coding for RNA or a protein. In contrast, “regulatory genes” are structural genes which encode products which control the expression of other genes (e.g., transcription factors).

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “label” or “detectable label” are used herein, to refer to any composition detectable by spectroscopic, photochemical, biochemical, immunochemical, electrical, optical or chemical means. Such labels include biotin for staining with labeled streptavidin conjugate, magnetic beads (e.g., Dynabeads®), fluorescent dyes (e.g., fluorescein, texas red, rhodamine, green fluorescent protein, and the like), radiolabels (e.g., ³H, ¹²⁵I, ³⁵ S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference). The labels contemplated in the present invention may be detected by many methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

The term “binding” as used herein, refers to any interaction between an infection control composition and a surface. Such as surface is defined as a “binding surface”. Binding may be reversible or irreversible. Such binding may be, but is not limited to, non-covalent binding, covalent bonding, ionic bonding, Van de Waal forces or friction, and the like. An infection control composition is bound to a surface if it is impregnated, incorporated, coated, in suspension with, in solution with, mixed with, etc.

### BRIEF DESCRIPTION OF THE DRAWINGS

The file of this patent contains at least one drawing executed in color. Copies of this patent with color drawings will be provided by the Patent and Trademark Office upon request and payment of the necessary fee.

FIG. 1 presents one embodiment of the concept of creating Hamming barcodes

FIG. 1A: Two representative Hamming hyperspheres (blue: center coordinates=(0, 0, 0); red: center coordinates=(1, 1, 1)).

FIG. 1B: Codeword regions comprising a length of 16 (or longer) checked by parity bits at positions 0, 1, 2, and 4: bits that are checked by each position are marked with 1.

FIG. 1C: Decoding a “received” codeword containing the binary value of 3 (0011) (n=7, k=4): Case 1: No errors. Case 2: Single-bit error at position 6 that is detected and corrected.

FIG. 2 presents exemplary data showing UniFrac clustering of samples from a cystic fibrosis lung, a Guerrero Negro microbial mat, air, and North American rivers obtained by pyrosequencing with barcodes.

FIG. 3 shows taxonomic distributions of bacteria in each of the major sample types in FIG. 2.

### DETAILED DESCRIPTION OF THE INVENTION

The present invention relates to nucleic acid sequencing. In particular, the invention relates to methods and compositions for detecting errors and correcting such errors during nucleic acid amplification (i.e., for example, a nucleic acid barcode) such that accurate sample identification may be maintained. The combination of the methods and compositions described herein allow characterization of a plurality of nucleic acid samples simultaneously when using high throughput amplification and/or sequencing technologies.

In one embodiment, the present invention contemplates a composition comprising a tagged (i.e., for example, a Hamming barcode) nucleotide sequence, wherein the nucleotide averages between approximately 270 nucleotides and 1500 nucleotides. In one embodiment, the nucleotide sequence is derived from the 16S rRNA gene. Other embodiments provide a tagged nucleotide sequence wherein the tag is attached to the 3′ or 5′ end of the nucleotide sequence. Alternatively, some embodiments of the present invention contemplate a tagged nucleotide sequence wherein the tag is attached to both the 3′ and 5′ ends of the nucleotide sequence. Although it is not necessary to understand the mechanism of an invention, it is believed that single end tags may be advantageous for sequencing because variation in the length of variable regions in different species may preclude the second tag from being read.

In one embodiment, the present invention contemplates a method comprising: a) amplifying a nucleic acid sample using a primer comprising a barcode; and b) using the barcodes to provide sample assignments to a sample from which the nucleic acid was obtained. Although it is not necessary to understand the mechanism of an invention, it is believed that such sample assignments can be done with high confidence because of the unique error-detecting/correcting barcodes that correct amplification mistakes in each respective sample, thereby maintaining the integrity of the sample assignment information.

### I. Conventional Error-Correcting Coding

The use of error-correction codes has been implemented in many different fields of art. For example, not only in biotechnology, but in information media such as cell phones and/or compact disks. R H Morelos-Zaragoza, The Art of Error-Correcting Coding. (John Wiley & Sons, Hoboken, N.J., (2006). As discussed below, these conventional techniques did not recognize, or employ, the advantages of Hamming barcodes (infra).

A. Cell Culture Assays

Quantitative and highly parallel methods for analyzing deletion mutants using barcodes in Saccharomyces cerevisiae have been reported. Shoemaker et al., “Quantitative Phenotypic Analysis of Yeast Deletion Mutants Using a Highly Parallel Molecular Bar-Coding Strategy” Nature Genetics 14(4): 450-456 (1996). This approach uses a PCR targeting strategy to generate large numbers of deletion strains that are individually labeled with a unique 20-base tag sequence that can be detected by hybridization to a high-density oligonucleotide array. The tags serve as unique identifiers (molecular barcodes) that allow analysis of large numbers of deletion strains simultaneously through selective growth conditions.

B. Vector Analysis Assays

Methods for identifying an mRNA source pool from which individual cDNAs were derived have been tried by adding unique 6-nucleotide “bar codes” to the 3′-end of each mRNA during first-strand cDNA synthesis. Qiu et al., “DNA Sequence-Based “Bar Codes” for Tracking the Origins of Expressed Sequence Tags From a Maize Library Constructed Using Multiple mRNA Sources” Plant Physiology 133: 475-481 (2003). This method utilized an error-correcting decoding algorithm that identified a source mRNA pool for more than 97% of the expressed sequence tags (ESTs) examined. Of the 3,684 sequences examined with this decoding algorithm, 3,531 (95.8%) had exact bar code matches, 70 (1.9%) had errors in their bar codes that were decodable, and 83 (2.3%) were not decodable.

This prior method relies upon a natural metric for designing DNA bar codes known as an “edit metric” where the minimal distance between two strands of bar code DNA sequences is a single base insertion, deletion, or substitution required to transform one strand into the other. (Gusfield, 1997). This method produces a higher rate of uncorrectable errors than other barcoded libraries, thus requiring bar codes that allow for the correction of two errors (i.e., for example, being at least five edits apart). To address this problem, it is pointed out that lengthening the bar codes by just 2 by (to 8 bp) would provide 34 unique bar codes (Ashlock et al., 2002). Unlike the present invention, these bar codes are located within an EST sequence by identifying the vector and poly(T) sequences and then determining whether the bases at the approximate location of the bar code match any of the bar codes used in the construction of the library.

C. Pyrosequencing Assays

Methods of labeling and amplifying nucleic acid molecules with primers comprising unique five-nucleotide barcodes have been identified following amplification by methods that include pyrosequencing. Ronaghi et al. “Methods and Compositions for Clonal Amplification of Nucleic Acid” United States Patent Application Number 2006/008,824 (herein incorporated by reference). The described barcoded primers are attached to a solid surface (i.e., for example, a bead) such that specific nucleic acid targets may be isolated/immobilized prior to amplification with other (non-barcoded) primers. While the resulting PCR product(s) include the unique barcode sequence the barcoded PCR primer(s) are not amplified.

DNA bar codes and pyro sequencing have been used to detect minor drug resistance mutations in multidrug-resistant HIV populations. Each primer consisted of the conventional 454 A and 454 B sequences at the 5′ ends and the HIV-complementary regions at the 3′ end separated by a 4-nucleotide DNA bar code sequence. The results identified a variety of minor drug resistance alleles in patient samples and demonstrated the feasibility of using pyrosequencing for efficient HIV genotyping. Several controls were included in these experiments to allow estimations of the background error rate associated with pyrosequencing. Hoffmann et al., “DNA Barcoding and Pyrosequencing to Identify Rare HIV Drug Resistance Mutations” Nucleic Acids Research 35(13): e91 (2007).

Pyrosequencing-tailored barcoding approaches have been reported that utilize 48 reverse-forward barcode pairs that are separated by a cloning linker, and are unique with respect to at least 4 nucleotide positions. Such a configuration was believed to provide uniquely barcoded libraries from up to 48 different samples. The barcoded primers were each 45-46 nucleotides long and consisted of: i) a forward or reverse 454 sequencing primer, ii) a forward or reverse barcode and iii) a forward or reverse cloning-linker. Lengthening the barcodes and/or increasing the variation(s) in the fixed forward and reverse linkers may expand the multiplexing capacity of this approach. Parameswaran et al., “A Pyrosequencing-Tailored Nucleotide Barcode Design Unveils Opportunities For Large-Scale Sample Multiplexing” Nucleic Acids Research 35(19): e130 (2007).

Conventional PCR with 5′-nucleotide tagged primers can generate homologous DNA amplification products from multiple specimens that are then subjected to pyrosequencing. Each DNA sequence is subsequently traced back to its individual source through 5′tag-analysis. This approach enables the assignment of virtually all the generated DNA sequences to the correct source once sequencing anomalies are accounted for. Binladen et al., “The Use of Coded PCR Primers Enables High-Throughput Sequencing of Multiple Homolog Amplification Products By 454 Parallel Sequencing” PloS ONE 2: e197 (2007). Conventional primers specific for 16S mammalian mitochondrial DNA (mtDNA) were modified into sixteen unique forward, and sixteen reverse primers through the addition of 5′-dinucleotide tags. The results indicated a bias in the distribution of the differently tagged primers that is dependent on the 5′ nucleotide of the tag. Specifically, primers 5′-labeled with a cytosine were heavily overrepresented among the final sequences, while those 5′-labeled with a thymine were strongly underrepresented. A weaker bias was also reported for the distribution of sequences sorted by the second nucleotide of the dinucleotide tags. In comparison to the dinucleotide tags, the performance of tetranucleotide tagged primers was less efficient than predicted. Although the small number of tetranucleotide tagged primers tested renders statistically supported comparisons difficult, data indicate that overall the rate of sequence miss-assignment for these primers was lower than for the dinucleotide tags.

Characterization of 141,000 sequences of 16S rRNA genes obtained from 100 uncultured gastrointestinal bacterial samples from rhesus macaques was performed using primers marked with a “unique DNA bar code”. These bar codes were represented by distinctive 4 base sequences between the 16S rRNA gene complementarity region and the pyrosequencing primer binding site. McKenna et al., “The Macaque Gut Microbiome In Health, Lentiviral Infection, and Chronic Enterocolitis” PloS Pathog. 4(2): e20 (2008). The resulting error rate for the barcoding procedure was estimated by cataloging all those sequences reads with bar codes that were not among those used for labeling. The analysis indicated that only 0.01% of sequences were likely to be miscataloged due to errors parsing the bar codes.

Integration site populations have been characterized from gene transfer studies using DNA barcoding and pyrosequencing. To sequence all the samples in a single sequencing experiment, primers that contain unique 4-bp barcodes were used in the second PCR step. The PCR products were gel purified and pooled prior to pyrosequencing. Wang et al., “DNA Barcoding and Pyrosequencing to Analyze Adverse Events In Therapeutic Gene Transfer” Nucleic Acids Research 36(9): e49 (2008).

454-pyrosequencing based methods have been reported for monitoring microbial communities in which the hyper-variable region of the 16S rRNA gene is amplified using primers that target adjacent conserved regions followed by direct sequencing of individual PCR products. Andersson et al., “Comparative Analysis of Human Gut Microbiota by Barcoded Pyrosequencing, PloS ONE 3(7): e2836 (2008). Including a sample-specific four nucleotide barcode sequence on one of the primers allows multiple samples to be analyzed in parallel on a single 454-pyrosequencing plate. It was suggested that the recognized pyrosequencing error rate might potentially disturb taxonomic classifications but offered not suggestions for using error correcting and/or detecting Hamming barcodes.

Methods that couple multiplex PCR with sample-specific DNA barcodes and “next-generation sequencing” (i.e., for example, pyrosequencing) have been reported to enable mutation discovery in candidate genes for multiple samples in parallel. The final amplification step of this method relies on universal PCR primers tailed with 454 Life Sciences A or B at the 5′ end, followed by a sample-specific DNA sequence and 454 sequencing primers such that the first few bases indicate from which sample each read originated. Varley et al., “Nested Patch PCR Enables Highly Multiplexed Mutation Discovery In Candidate Genes” Genome Res. 18:1844-1850 (2008). While the method was admittedly error-prone due to the nature of 454 sequencing, there was no suggestions to use error-correcting and/or detecting Hamming barcodes.

### II. Calculation Of Hamming Code Resolution

One class of error-correcting codes that use redundancy and standard linear algebra techniques has been referred to as a Hamming code. Hamming R. W., Bell System Technical Journal 29:147 (1950). Other encoding schemes similar to Hamming codes include Golay codes. Briefly, Hamming codes, like other error-correcting codes, are based on the principle of redundancy and are constructed by adding redundant parity bits to data that is to be transmitted over a noisy medium. Such error-correcting codes encode sample identifiers with redundant parity bits, and “transmit” these sample identifiers as codewords. Although it is not necessary to understand the mechanism of an invention, it is believed that if each nucleotide base is encoded by two (2) bits, then an eight (8) nucleotide base codeword would comprise sixteen (16) bits of information for transmission.

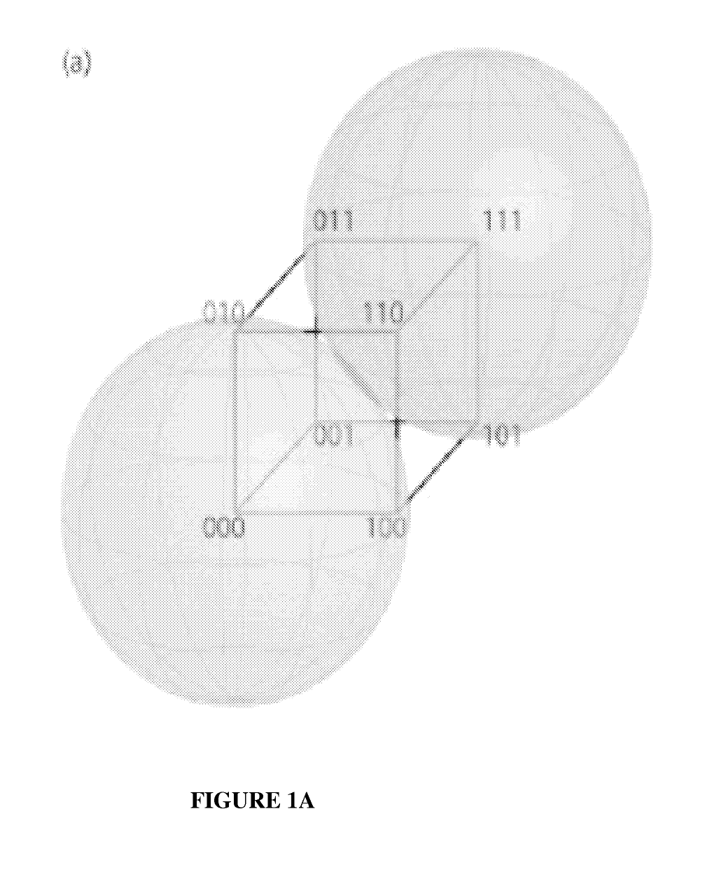

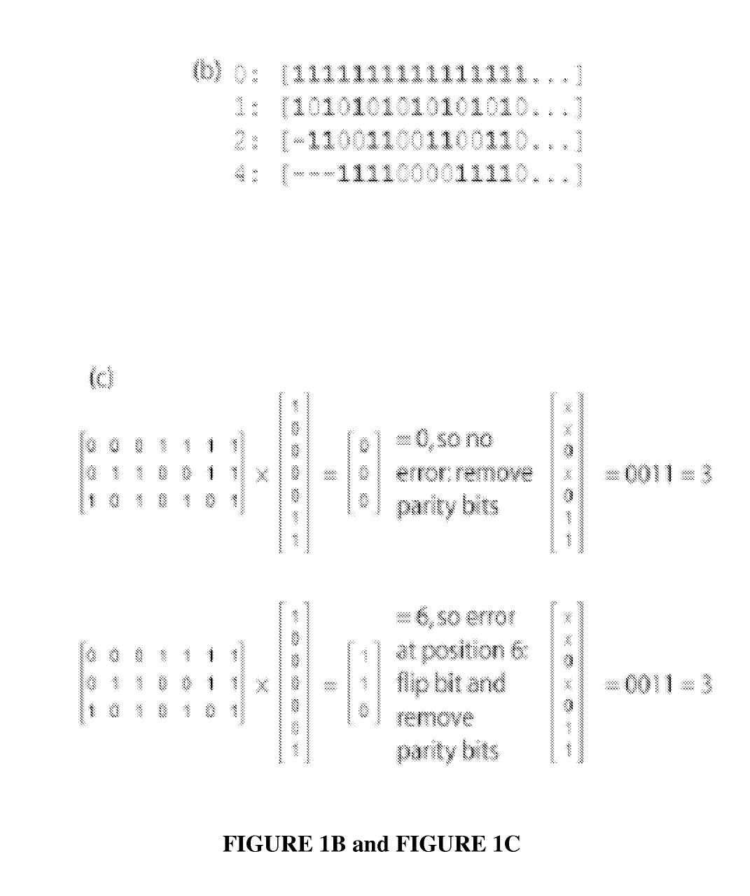

Hamming codes may be represented by a subset of the possible codewords that are chosen from the center of multidimensional spheres (i.e., for example, hyperspheres) in a binary subspace. Single bit errors may fall within hyperspheres associated with a specific codeword and can thus be corrected. On the other hand, double bit errors that do not associate with a specific codeword can be detected, but not corrected. Consider a first hypersphere centered at coordinates (0, 0, 0) (i.e., for example, using an x-y-z coordinate system), wherein any single-bit error can be corrected by falling within a radius of 1 from the center coordinates; i.e., for example, single bit errors having the coordinates of (0, 0, 0); (0, 1, 0); (0, 0, 1); (1, 0, 0), or (1, 1, 0). Likewise, a second hypersphere may be constructed wherein single-bit errors can be corrected by falling within a radius of 1 of its center coordinates (1, 1, 1) (i.e., for example, (1,1,1); (1, 0, 1); (0 ,1, 0); or (0, 1, 1). See, FIG. 1A (first hypersphere-blue; second hypersphere-red).

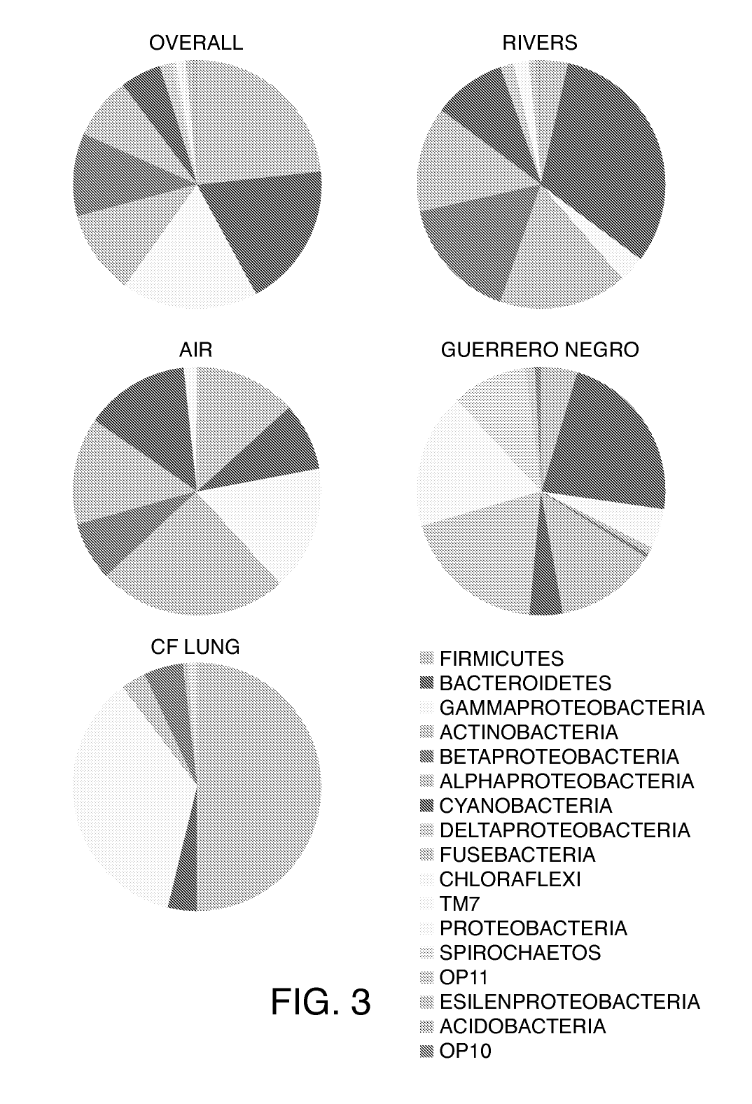

Codeword regions comprising a length of 16 or more bits may be checked by parity bits at positions 0, 1, 2, and 4, wherein the bits that are checked by each position are marked with 1. See, FIG. 1B. Consequently, a “received” codeword containing a binary value of 3 (0011) (n=7, k=4) may be decoded for possible correction. The first case contains no errors; the second contains a single-bit error at position 6 that is detected and corrected. See, FIG. 3. Note that this is an example of a Hamming error-correcting code: the method claims all error-detecting and error-correcting codes.

For example, let n be the total number of bits in the codeword being transmitted, and k be the number of bits of information to be transmitted. Hamming codes use n-k bits of redundancy, and because not all 2ⁿ possible codewords are used, there are 2^{k} valid, error-correcting codewords is 2^{k} that form a k-dimensional subspace. The Hamming distance is defined as the number of bits that differ between two vectors in this subspace, and the relevant parameter for error-correction is the minimum Hamming distance. Next, let t be the radius of a sphere in this subspace where any change within this sphere can be corrected. The error-correcting capability is the largest radius such that all Hamming spheres are disjoint: t=floor((dₘᵢₙ−1)/2), where dₘᵢₙ is the minimum Hamming distance. Thus, the minimum Hamming distance between codewords needed to correct a single error is 3.

In one embodiment, the present invention contemplates a barcode that uses Hamming codes to encode sample identifiers as DNA translations of each binary codeword using 2 bits/base. For example, 8-base codewords (n=16) use 11 bits for sample identifiers (k=11), and 5 bits of redundancy (n−k=5). There are thus 2¹¹=2048 possible 8-base codewords. Alternatively, a 4-base barcodes can encode up to 16 codewords, thereby generating 67 million 16-base codewords. One can easily using increasing base lengths to provide ready scalability.

### III. Error-Correction Hamming Codes in Pyrosequencing

Pyrosequencing may improve sequencing by eliminating the laborious step of producing clone libraries and generating hundreds of thousands of sequences in a single run. Margulies et al., Nature 437(7057):376 (2005). These improvements may include, for example, the ability to assess global microbial community diversity Huber et al., Science 318(5847):97 (2007); Roesch et al., ISME J 1:283 (2007); Sogin et al., Proc Natl Acad Sci USA 103:12115 (2006). In one embodiment, the present invention contemplates a method comprising pyrosequencing amplified nucleic acids containing Hamming barcoded error-correcting and/or error-detecting primers. In one embodiment, the method further comprises estimating the total sequencing error rate. In one embodiment, the method further comprises eliminating sample mis-assignment of the nucleic acid.

In one embodiment, the present invention contemplates a method comprising amplifying nucleic acids. In one embodiment, the amplification method may further comprise steps including, but not limited to, sequencing genes, detecting alleles, or diagnosing a medical condition. Further, a nucleic acid amplification method may comprise detecting and/or correcting nucleotide sequence errors as a research tool for understanding of microbial habitats.

The presently disclosed methods have several advantages over conventionally used pyrosequencing methods currently in use including, but not limited to: 1) the ability to detect and correct errors in the barcodes to eliminate possible mis-assignment; 2) the barcodes only require 8 nucleotides, which is important when read lengths are limited; and 3) the ability to tag only one end of the sequence (i.e., for example, tagging the reverse primer) is useful since variation in the length of variable regions in different species may preclude a second tag from being read.

Conventional culture-independent 16S rRNA-based analysis of microbial community composition through pyrosequencing has been limited by the expense of each individual run, and by the difficulty of splitting a single plate across multiple runs. N. R. Pace, Science 276(5313): 734 (1997). Several reports have suggested that a barcode (i.e., a unique tag) may be added to each primer before PCR amplification. Binladen et al., PLoS ONE 2 (2), e197 (2007): Hoffmann et al., Nucleic Acids Res 35 (13), e91 (2007); and Parameswaran et al., Nucleic Acids Res 35 (19), e130 (2007). In one embodiment, the present invention contemplates a method comprising amplifying each sample with a known tagged primer, wherein the subsequent sequencing can be performed on an equimolar mixture of PCR-amplified DNA from each sample, thereby allowing the sequences to be assigned to samples based on the unique barcode.

Disadvantages of such conventional pyrosequencing barcoding methods (supra) include, but are not limited to: i) sequencing only twenty-five samples in a single pyrosequencing run; ii) a limited number of usable unique barcodes; or iii) an ability to detect sequencing errors that change sample assignment and/or identification. Although it is not necessary to understand the mechanism of an invention, it is believed that overcoming these disadvantages by using pyrosequencing in conjunction with Hamming barcodes will create a highly robust method that maintains an error-free sample assignment code. For example, because the 5′ end of the read is generally considered more error-prone than other nucleotide regions the presently disclosed invention is believed to solve this problem. Huse et al., Genome Biol 8:R143 (2007).

A. Identifying Nucleic Acid Sequences Tagged with Bar Codes

In one embodiment, the present invention contemplates an improved method for culture-independent 16S rRNA pyrosequencing analysis that reduces both cost and error rate by processing more than 25 samples in a single pyrosequencing run. PCR amplification of each sample with unique barcode tagged primers prior to pyrosequencing permits an assignment of sequence data to individual samples from equimolar mixtures of PCR-amplified DNA.

In one embodiment, the present invention contemplates a barcode based on error-correcting Hamming codes that use a minimum amount of redundancy and are implemented using standard linear algebraic techniques. In addition to increasing the numbers of unique barcodes available, error-correcting barcodes are able to detect and/or correct sequencing errors. Although it is not necessary to understand the mechanism of an invention, it is believed that such sequencing errors occurring within a barcode are sufficient to change sample identification assignments. This technique is readily scalable, for example while an 8-base barcode upon which the present primers were created provide 2,048 possible combinations, a 4-base barcode would provide 16 possible combinations, and a 16-base barcode would provide 67 million possible combinations.

In one embodiment, the present invention contemplates using a Hamming code analysis to identify an 8-base barcode scheme using the nucleotides including but not limited to, adenosine (A), thymidine (T), cytosine (C), or guano sine (G) (i.e., for example, at least 1544 barcodes). See, Table 1.

TABLE 1
Representative 8-Nucleotide Base
Error-Correcting Barcodes And
Representative Primer Sequence

| Barcode | Primer |
|---|---|
| AACCAACC | GCTCCCTCGCGCCATCAGAACCAACCCATGCTC |
| SEQ ID NO: 1 | GCCTCCCGTAGGAGT |
| | SEQ ID NO: 2 |
| AACCAAGG | GCCTCCCTCGCGCCATCAGAACCAAGGCATGCT |
| SEQ ID NO: 3 | GCCTCCCGTAGGAGT |
| | SEQ ID NO: 4 |
| AACCATCG | GCCTCCCTCGCGCCATCAGAACCATCGCATGCT |
| SEQ ID NO: 5 | GCCTCCCGTAGGAGT |
| | SEQ ID NO: 6 |
| AACCATGC | GCCTCCCTCGCGCCATCAGAACCATGCCATGCT |
| SEQ ID NO: 7 | GCCTCCCGTAGGAGT |
| | SEQ ID NO: 8 |
| AACCGCAT | GCCTCCCTCGCGCCATCAGAACCGCATCATGCT |
| SEQ ID NO: 9 | GCCTCCCGTAGGAGT |
| | SEQ ID NO: 10 |
| AACCGCTA | GCCTCCCTCGCGCCATCAGAACCGCTACATGCT |
| SEQ ID NO: 11 | GCCTCCCGTAGGAGT |
| | SEQ ID NO: 12 |
| AACCGGAA | GCCTCCCTCGCGCCATCAGAACCGGAACATGCT |
| SEQ ID NO: 13 | GCCTCCCGTAGGAGT |
| | SEQ ID NO: 14 |
| AACCGGTT | GCCTCCCTCGCGCCATCAGAACCGGTTCATGCT |
| SEQ ID NO: 15 | GCCTCCCGTAGGAGT |
| | SEQ ID NO: 16 |
| AACCTACG | GCCTCCCTCGCGCCATCAGAACCTACGCATGCT |
| SEQ ID NO: 17 | GCCTCCCGTAGGAGT |
| | SEQ ID NO: 18 |
| AACCTAGC | GCCTCCCTCGCGCCATCAGAACCTAGCCATGCT |
| SEQ ID NO: 19 | GCCTCCCGTAGGAGT |
| | SEQ ID NO: 20 |
| AACCTTCC | GCCTCCCTCGCGCCATCAGAACCTTCCCATGCT |
| SEQ ID NO: 21 | GCCTCCCGTAGGAGT |
| | SEQ ID NO: 22 |
| AACCTTGG | GCCTCCCTCGCGCCATCAGAACCTTGGCATGCT |
| SEQ ID NO: 23 | GCCTCCCGTAGGAGT |
| | SEQ ID NO: 24 |
| AACGAACG | GCCTCCCTCGCGCCATCAGAACGAACGCATGCT |
| SEQ ID NO: 25 | GCCTCCCGTAGGAGT |
| | SEQ ID NO: 26 |
| AACGAAGC | GCCTCCCTCGCGCCATCAGAACGAAGCCATGCT |
| SEQ ID NO: 27 | GCCTCCCGTAGGAGT |
| | SEQ ID NO: 28 |
| AACGATCC | GCCTCCCTCGCGCCATCAGAACGATCCCATGCT |
| SEQ ID NO: 29 | GCCTCCCGTAGGAGT |
| | SEQ ID NO: 30 |
| AACGATGG | GCCTCCCTCGCGCCATCAGAACGATGGCATGCT |
| SEQ ID NO: 31 | GCCTCCCGTAGGAGT |
| | SEQ ID NO: 32 |
| AACGCCAT | GCCTCCCTCGCGCCATCAGAACGCCATCATGCT |
| SEQ ID NO: 33 | GCCTCCCGTAGGAGT |
| | SEQ ID NO: 34 |
| AACGCCTA | GCCTCCCTCGCGCCATCAGAACGCCTACATGCT |
| SEQ ID NO: 35 | GCCTCCCGTAGGAGT |
| | SEQ ID NO: 36 |
| AACGCGAA | GCCTCCCTCGCGCCATCAGAACGCGAACATGCT |
| SEQ ID NO: 37 | GCCTCCCGTAGGAGT |
| | SEQ ID NO: 38 |
| AACGCGTT | GCCTCCCTCGCGCCATCAGAACGCGTTCATGCT |
| SEQ ID NO: 39 | GCCTCCCGTAGGAGT |
| | SEQ ID NO: 40 |
| AACGGCAA | GCCTCCCTCGCGCCATCAGAACGGCAACATGCT |
| SEQ ID NO: 41 | GCCTCCCGTAGGAGT |
| | SEQ ID NO: 42 |
| AACGGCTT | GCCTCCCTCGCGCCATCAGAACGGCTTCATGCT |
| SEQ ID NO: 43 | GCCTCCCGTAGGAGT |
| | SEQ ID NO: 44 |
| AACGTACC | GCCTCCCTCGCGCCATCAGAACGTACCCATGCT |
| SEQ ID NO: 45 | GCCTCCCGTAGGAGT |
| | SEQ ID NO: 46 |
| AACGTAGG | GCCTCCCTCGCGCCATCAGAACGTAGGCATGCT |
| SEQ ID NO: 47 | CTCCCGTAGGAGT |
| | SEQ ID NO: 48 |
| AACGTTCG | GCCTCCCTCGCGCCATCAGAACGTTCGCATGCT |
| SEQ ID NO: 49 | GCCTCCCGTAGGAGT |
| | SEQ ID NO: 50 |
| AACGTTGC | GCCTCCCTCGCGCCATCAGAACGTTGCCATGCT |
| SEQ ID NO: 51 | GCCTCCCGTAGGAGT |
| | SEQ ID NO: 52 |
| AAGCAACG | GCCTCCCTCGCGCCATCAGAAGCAACGCATGCT |
| SEQ ID NO: 53 | GCCTCCCGTAGGAGT |
| | SEQ ID NO: 54 |
| AAGCAAGC | GCCTCCCTCGCGCCATCAGAAGCAAGCCATGCT |
| SEQ ID NO: 55 | GCCTCCCGTAGGAGT |
| | SEQ ID NO: 56 |
| AAGCATCC | GCCTCCCTCGCGCCATCAGAAGCATCCCATGCT |
| SEQ ID NO: 57 | GCCTCCCGTAGGAGT |
| | SEQ ID NO: 58 |
| AAGCATGG | GCCTCCCTCGCGCCATCAGAAGCATGGCATGCT |
| SEQ ID NO: 59 | GCCTCCCGTAGGAGT |
| | SEQ ID NO: 60 |
| AAGCCGAA | GCCTCCCTCGCGCCATCAGAAGCCGAACATGCT |
| SEQ ID NO: 61 | GCCTCCCGTAGGAGT |
| | SEQ ID NO: 62 |
| AAGCCGTT | GCCTCCCTCGCGCCATCAGAAGCCGTTCATGCT |
| SEQ ID NO: 63 | GCCTCCCGTAGGAGT |
| | SEQ ID NO: 64 |
| AAGCGCAA | GCCTCCCTCGCGCCATCAGAAGCGCAACATGCT |
| SEQ ID NO: 65 | GCCTCCCGTAGGAGT |
| | SEQ ID NO: 66 |
| AAGCGCTT | GCCTCCCTCGCGCCATCAGAAGCGCTTCATGCT |
| SEQ ID NO: 67 | GCCTCCCGTAGGAGT |
| | SEQ ID NO: 68 |
| AAGCGGAT | GCCTCCCTCGCGCCATCAGAAGCGGATCATGCT |
| SEQ ID NO: 69 | GCCTCCCGTAGGAGT |
| | SEQ ID NO: 70 |
| AAGCGGTA | GCCTCCCTCGCGCCATCAGAAGCGGTACATGCT |
| SEQ ID NO: 71 | GCCTCCCGTAGGAGT |
| | SEQ ID NO: 72 |
| AAGCTACC | GCCTCCCTCGCGCCATCAGAAGCTACCCATGCT |
| SEQ ID NO: 73 | GCCTCCCGTAGGAGT |
| | SEQ ID NO: 74 |
| AAGCTAGG | GCCTCCCTCGCGCCATCAGAAGCTAGGCATGCT |
| SEQ ID NO: 75 | GCCTCCCGTAGGAGT |
| | SEQ ID NO: 76 |
| AAGCTTCG | GCCTCCCTCGCGCCATCAGAAGCTTCGCATGCT |
| SEQ ID NO: 77 | GCCTCCCGTAGGAGT |
| | SEQ ID NO: 78 |
| AAGCTTGC | GCCTCCCTCGCGCCATCAGAAGCTTGCCATGCT |
| SEQ ID NO: 79 | GCCTCCCGTAGGAGT |
| | SEQ ID NO: 80 |
| AAGGAACC | GCCTCCCTCGCGCCATCAGAAGGAACCCATGCT |
| SEQ ID NO: 81 | GCCTCCCGTAGGAGT |
| | SEQ ID NO: 82 |
| AAGGAAGG | GCCTCCCTCGCGCCATCAGAAGGAAGGCATGCT |
| SEQ ID NO: 83 | GCCTCCCGTAGGAGT |
| | SEQ ID NO: 84 |
| AAGGATCG | GCCTCCCTCGCGCCATCAGAAGGATCGCATGCT |
| SEQ ID NO: 85 | GCCTCCCGTAGGAGT |
| | SEQ ID NO: 86 |
| AAGGATGC | GCCTCCCTCGCGCCATCAGAAGGATGCCATGCT |
| SEQ ID NO: 87 | GCCTCCCGTAGGAGT |
| | SEQ ID NO: 88 |
| AAGGCCAA | GCCTCCCTCGCGCCATCAGAAGGCCAACATGCT |
| SEQ ID NO: 89 | GCCTCCCGTAGGAGT |
| | SEQ ID NO: 90 |
| AAGGCCTT | GCCTCCCTCGCGCCATCAGAAGGCCTTCATGCT |
| SEQ ID NO: 91 | GCCTCCCGTAGGAGT |
| | SEQ ID NO: 92 |
| AAGGCGAT | GCCTCCCTCGCGCCATCAGAAGGCGATCATGCT |
| SEQ ID NO: 93 | GCCTCCCGTAGGAGT |
| | SEQ ID NO: 94 |
| AAGGCGTA | GCCTCCCTCGCGCCATCAGAAGGCGTACATGCT |
| SEQ ID NO: 95 | GCCTCCCGTAGGAGT |
| | SEQ ID NO: 96 |
| AAGGTACG | GCCTCCCTCGCGCCATCAGAAGGTACGCATGCT |
| SEQ ID NO: 97 | GCCTCCCGTAGGAGT |
| | SEQ ID NO: 98 |
| AAGGTAGC | GCCTCCCTCGCGCCATCAGAAGGTAGCCATGCT |
| SEQ ID NO: 99 | GCCTCCCGTAGGAGT |
| | SEQ ID NO: 100 |
| AAGGTTCC | GCCTCCCTCGCGCCATCAGAAGGTTCCCATGCT |
| SEQ ID NO: 101 | GCCTCCCGTAGGAGT |
| | SEQ ID NO: 102 |
| AAGGTTGG | GCCTCCCTCGCGCCATCAGAAGGTTGGCATGCT |
| SEQ ID NO: 103 | GCCTCCCGTAGGAGT |
| | SEQ ID NO: 104 |
| AATACCGC | GCCTCCCTCGCGCCATCAGAATACCGCCATGCT |
| SEQ ID NO: 104 | GCCTCCCGTAGGAGT |
| | SEQ ID NO: 106 |
| AATACGCC | GCCTCCCTCGCGCCATCAGAATACGCCCATGCT |
| SEQ ID NO: 107 | GCCTCCCGTAGGAGT |
| | SEQ ID NO: 108 |
| AATAGCGG | GCCTCCCTCGCGCCATCAGAATAGCGGCATGCT |
| SEQ ID NO: 109 | GCCTCCCGTAGGAGT |
| | SEQ ID NO: 110 |
| AATAGGCG | GCCTCCCTCGCGCCATCAGAATAGGCGCATGCT |
| SEQ ID NO: 111 | GCCTCCCGTAGGAGT |
| | SEQ ID NO: 112 |
| AATTCCGG | GCCTCCCTCGCGCCATCAGAATTCCGGCATGCT |
| SEQ ID NO: 113 | GCCTCCCGTAGGAGT |
| | SEQ ID NO: 114 |
| AATTCGCG | GCCTCCCTCGCGCCATCAGAATTCGCGCATGCT |
| SEQ ID NO: 115 | GCCTCCCGTAGGAGT |
| | SEQ ID NO: 116 |
| AATTCGGC | GCCTCCCTCGCGCCATCAGAATTCGGCCATGCT |
| SEQ ID NO: 117 | GCCTCCCGTAGGAGT |
| | SEQ ID NO: 118 |
| AATTGCCG | GCCTCCCTCGCGCCATCAGAATTGCCGCATGCT |
| SEQ ID NO: 119 | GCCTCCCGTAGGAGT |
| | SEQ ID NO: 120 |
| AATTGCGC | GCCTCCCTCGCGCCATCAGAATTGCGCCATGCT |
| SEQ ID NO: 121 | GCCTCCCGTAGGAGT |
| | SEQ ID NO: 122 |
| AATTGGCC | GCCTCCCTCGCGCCATCAGAATTGGCCCATGCT |
| SEQ ID NO: 123 | GCCTCCCGTAGGAGT |
| | SEQ ID NO: 124 |
| ACACACAC | GCCTCCCTCGCGCCATCAGACACACACCATGCT |
| SEQ ID NO: 125 | GCCTCCCGTAGGAGT |
| | SEQ ID NO: 126 |
| ACACACTG | GCCTCCCTCGCGCCATCAGACACACTGCATGCT |
| SEQ ID NO: 127 | GCCTCCCGTAGGAGT |
| | SEQ ID NO: 128 |
| ACACAGAG | GCCTCCCTCGCGCCATCAGACACAGAGCATGCT |
| SEQ ID NO: 129 | GCCTCCCGTAGGAGT |
| | SEQ ID NO: 130 |
| ACACAGTC | GCCTCCCTCGCGCCATCAGACACAGTCCATGCT |
| SEQ ID NO: 131 | GCCTCCCGTAGGAGT |
| | SEQ ID NO: 132 |
| ACACCACA | GCCTCCCTCGCGCCATCAGACACCACACATGCT |
| SEQ ID NO: 133 | GCCTCCCGTAGGAGT |
| | SEQ ID NO: 134 |
| ACACCAGT | GCCTCCCTCGCGCCATCAGACACCAGTCATGCT |
| SEQ ID NO: 135 | GCCTCCCGTAGGAGT |
| | SEQ ID NO: 136 |
| ACACCTCT | GCCTCCCTCGCGCCATCAGACACCTCTCATGCT |
| SEQ ID NO: 137 | GCCTCCCGTAGGAGT |
| | SEQ ID NO: 138 |
| ACACCTGA | GCCTCCCTCGCGCCATCAGACACCTGACATGCT |
| SEQ ID NO: 139 | GCCTCCCGTAGGAGT |
| | SEQ ID NO: 140 |
| ACACGACT | GCCTCCCTCGCGCCATCAGACACGACTCATGCT |
| SEQ ID NO: 141 | GCCTCCCGTAGGAGT |
| | SEQ ID NO: 142 |
| ACACGAGA | GCCTCCCTCGCGCCATCAGACACGAGACATGCT |
| SEQ ID NO: 143 | GCCTCCCGTAGGAGT |
| | SEQ ID NO: 144 |
| ACACGTCA | GCCTCCCTCGCGCCATCAGACACGTCACATGCT |
| SEQ ID NO: 145 | GCCTCCCGTAGGAGT |
| | SEQ ID NO: 146 |
| ACACGTGT | GCCTCCCTCGCGCCATCAGACACGTGTCATGCT |
| SEQ ID NO: 147 | GCCTCCCGTAGGAGT |
| | SEQ ID NO: 148 |
| ACACTCAG | GCCTCCCTCGCGCCATCAGACACTCAGCATGCT |
| SEQ ID NO: 149 | GCCTCCCGTAGGAGT |
| | SEQ ID NO: 150 |
| ACACTCTC | GCCTCCCTCGCGCCATCAGACACTCTCCATGCT |
| SEQ ID NO: 151 | GCCTCCCGTAGGAGT |
| | SEQ ID NO: 152 |
| ACACTGAC | GCCTCCCTCGCGCCATCAGACACTGACCATGCT |
| SEQ ID NO: 153 | GCCTCCCGTAGGAGT |
| | SEQ ID NO: 154 |
| ACACTGTG | GCCTCCCTCGCGCCATCAGACACTGTGCATGCT |
| SEQ ID NO: 155 | GCCTCCCGTAGGAGT |
| | SEQ ID NO: 156 |
| ACAGACAG | GCCTCCCTCGCGCCATCAGACAGACAGCATGCT |
| SEQ ID NO: 157 | GCCTCCCGTAGGAGT |
| | SEQ ID NO: 158 |
| ACAGACTC | GCCTCCCTCGCGCCATCAGACAGACTCCATGCT |
| SEQ ID NO: 159 | GCCTCCCGTAGGAGT |
| | SEQ ID NO: 160 |
| ACAGAGAC | GCCTCCCTCGCGCCATCAGACAGAGACCATGCT |
| SEQ ID NO: 161 | GCCTCCCGTAGGAGT |
| | SEQ ID NO: 162 |
| ACAGAGTG | GCCTCCCTCGCGCCATCAGACAGAGTGCATGCT |
| SEQ ID NO: 163 | GCCTCCCGTAGGAGT |
| | SEQ ID NO: 164 |
| ACAGCACT | GCCTCCCTCGCGCCATCAGACAGCACTCATGCT |
| SEQ ID NO: 165 | GCCTCCCGTAGGAGT |
| | SEQ ID NO: 166 |
| ACAGCAGA | GCCTCCCTCGCGCCATCAGACAGCAGACATGCT |
| SEQ ID NO: 167 | GCCTCCCGTAGGAGT |
| | SEQ ID NO: 168 |
| ACAGCTCA | GCCTCCCTCGCGCCATCAGACAGCTCACATGCT |
| SEQ ID NO: 169 | GCCTCCCGTAGGAGT |
| | SEQ ID NO: 170 |
| ACAGCTGT | GCCTCCCTCGCGCCATCAGACAGCTGTCATGCT |
| SEQ ID NO: 171 | GCCTCCCGTAGGAGT |
| | SEQ ID NO: 172 |
| ACAGGACA | GCCTCCCTCGCGCCATCAGACAGGACACATGCT |
| SEQ ID NO: 173 | GCCTCCCGTAGGAGT |
| | SEQ ID NO: 174 |
| ACAGGAGT | GCCTCCCTCGCGCCATCAGACAGGAGTCATGCT |
| SEQ ID NO: 175 | GCCTCCCGTAGGAGT |
| | SEQ ID NO: 176 |
| ACAGGTCT | GCCTCCCTCGCGCCATCAGACAGGTCTCATGCT |
| SEQ ID NO: 177 | GCCTCCCGTAGGAGT |
| | SEQ ID NO: 178 |
| ACAGGTGA | GCCTCCCTCGCGCCATCAGACAGGTGACATGCT |
| SEQ ID NO: 179 | GCCTCCCGTAGGAGT |
| | SEQ ID NO: 180 |
| ACAGTCAC | GCCTCCCTCGCGCCATCAGACAGTCACCATGCT |
| SEQ ID NO: 181 | GCCTCCCGTAGGAGT |
| | SEQ ID NO: 182 |
| ACAGTCTG | GCCTCCCTCGCGCCATCAGACAGTCTGCATGCT |
| SEQ ID NO: 183 | GCCTCCCGTAGGAGT |
| | SEQ ID NO: 184 |
| ACAGTGAG | GCCTCCCTCGCGCCATCAGACAGTGAGCATGCT |
| SEQ ID NO: 185 | GCCTCCCGTAGGAGT |
| | SEQ ID NO: 186 |
| ACAGTGTC | GCCTCCCTCGCGCCATCAGACAGTGTCCATGCT |
| SEQ ID NO: 187 | GCCTCCCGTAGGAGT |
| | SEQ ID NO: 188 |
| ACCAACCA | GCCTCCCTCGCGCCATCAGACCAACCACATGCT |
| SEQ ID NO: 189 | GCCTCCCGTAGGAGT |
| | SEQ ID NO: 190 |
| ACCAACGT | GCCTCCCTCGCGCCATCAGACCAACGTCATGCT |
| SEQ ID NO: 191 | GCCTCCCGTAGGAGT |
| | SEQ ID NO: 192 |
| ACCAAGCT | GCCTCCCTCGCGCCATCAGACCAAGCTCATGCT |
| SEQ ID NO: 193 | GCCTCCCGTAGGAGT |
| | SEQ ID NO: 194 |
| ACCAAGGA | GCCTCCCTCGCGCCATCAGACCAAGGACATGCT |
| SEQ ID NO: 195 | GCCTCCCGTAGGAGT |
| | SEQ ID NO: 196 |
| ACCACAAC | GCCTCCCTCGCGCCATCAGACCACAACCATGCT |
| SEQ ID NO: 197 | GCCTCCCGTAGGAGT |
| | SEQ ID NO: 198 |
| ACCACATG | GCCTCCCTCGCGCCATCAGACCACATGCATGCT |
| SEQ ID NO: 199 | GCCTCCCGTAGGAGT |
| | SEQ ID NO: 200 |
| ACCACTAG | GCCTCCCTCGCGCCATCAGACCACTAGCATGCT |
| SEQ ID NO: 201 | GCCTCCCGTAGGAGT |
| | SEQ ID NO: 202 |
| ACCACTTC | GCCTCCCTCGCGCCATCAGACCACTTCCATGCT |
| SEQ ID NO: 203 | GCCTCCCGTAGGAGT |
| | SEQ ID NO: 204 |
| ACCAGAAG | GCCTCCCTCGCGCCATCAGACCAGAAGCATGCT |
| SEQ ID NO: 205 | GCCTCCCGTAGGAGT |
| | SEQ ID NO: 206 |
| ACCAGATC | GCCTCCCTCGCGCCATCAGACCAGATCCATGCT |
| SEQ ID NO: 207 | GCCTCCCGTAGGAGT |
| | SEQ ID NO: 208 |
| ACCAGTAC | GCCTCCCTCGCGCCATCAGACCAGTACCATGCT |
| SEQ ID NO: 209 | GCCTCCCGTAGGAGT |
| | SEQ ID NO: 210 |
| ACCAGTTG | GCCTCCCTCGCGCCATCAGACCAGTTGCATGCT |
| SEQ ID NO: 211 | GCCTCCCGTAGGAGT |
| | SEQ ID NO: 212 |
| ACCATCCT | GCCTCCCTCGCGCCATCAGACCATCCTCATGCT |
| SEQ ID NO: 213 | GCCTCCCGTAGGAGT |
| | SEQ ID NO: 214 |
| ACCATCGA | GCCTCCCTCGCGCCATCAGACCATCGACATGCT |
| SEQ ID NO: 215 | GCCTCCCGTAGGAGT |
| | SEQ ID NO: 216 |
| ACCATGCA | GCCTCCCTCGCGCCATCAGACCATGCACATGCT |
| SEQ ID NO: 217 | GCCTCCCGTAGGAGT |
| | SEQ ID NO: 218 |
| ACCATGGT | GCCTCCCTCGCGCCATCAGACCATGGTCATGCT |
| SEQ ID NO: 219 | GCCTCCCGTAGGAGT |
| | SEQ ID NO: 220 |
| ACCTACCT | GCCTCCCTCGCGCCATCAGACCTACCTCATGCT |
| SEQ ID NO: 221 | GCCTCCCGTAGGAGT |
| | SEQ ID NO: 222 |
| ACCTACGA | GCCTCCCTCGCGCCATCAGACCTACGACATGCT |
| SEQ ID NO: 223 | GCCTCCCGTAGGAGT |
| | SEQ ID NO: 224 |
| ACCTAGCA | GCCTCCCTCGCGCCATCAGACCTAGCACATGCT |
| SEQ ID NO: 225 | GCCTCCCGTAGGAGT |
| | SEQ ID NO: 226 |
| ACCTAGGT | GCCTCCCTCGCGCCATCAGACCTAGGTCATGCT |
| SEQ ID NO: 227 | GCCTCCCGTAGGAGT |
| | SEQ ID NO: 228 |
| ACCTCAAG | GCCTCCCTCGCGCCATCAGACCTCAAGCATGCT |
| SEQ ID NO: 229 | GCCTCCCGTAGGAGT |
| | SEQ ID NO: 230 |
| ACCTCATC | GCCTCCCTCGCGCCATCAGACCTCATCCATGCT |
| SEQ ID NO: 231 | GCCTCCCGTAGGAGT |
| | SEQ ID NO: 232 |
| ACCTCTAC | GCCTCCCTCGCGCCATCAGACCTCTACCATGCT |
| SEQ ID NO: 233 | GCCTCCCGTAGGAGT |
| | SEQ ID NO: 234 |
| ACCTCTTG | GCCTCCCTCGCGCCATCAGACCTCTTGCATGCT |
| SEQ ID NO: 235 | GCCTCCCGTAGGAGT |
| | SEQ ID NO: 236 |
| ACCTGAAC | GCCTCCCTCGCGCCATCAGACCTGAACCATGCT |
| SEQ ID NO: 237 | GCCTCCCGTAGGAGT |
| | SEQ ID NO: 238 |
| ACCTGATG | GCCTCCCTCGCGCCATCAGACCTGATGCATGCT |
| SEQ ID NO: 239 | GCCTCCCGTAGGAGT |
| | SEQ ID NO: 240 |
| ACCTGTAG | GCCTCCCTCGCGCCATCAGACCTGTAGCATGCT |
| SEQ ID NO: 241 | GCCTCCCGTAGGAGT |
| | SEQ ID NO: 242 |
| ACCTGTTC | GCCTCCCTCGCGCCATCAGACCTGTTCCATGCT |

| | |
|---|---|
| SEQ ID NO: 243 | GCCTCCCGTAGGAGT |
| | SEQ ID NO: 244 |
| ACCTTCCA | GCCTCCCTCGCGCCATCAGACCTTCCACATGCT |
| SEQ ID NO: 245 | GCCTCCCGTAGGAGT |
| | SEQ ID NO: 246 |
| ACCTTCGT | GCCTCCCTCGCGCCATCAGACCTTCGTCATGCT |
| SEQ ID NO: 247 | GCCTCCCGTAGGAGT |
| | SEQ ID NO: 248 |
| ACCTTGCT | GCCTCCCTCGCGCCATCAGACCTTGCTCATGCT |
| SEQ ID NO: 249 | GCCTCCCGTAGGAGT |
| | SEQ ID NO: 250 |
| ACCTTGGA | GCCTCCCTCGCGCCATCAGACCTTGGACATGCT |
| SEQ ID NO: 251 | GCCTCCCGTAGGAGT |
| | SEQ ID NO: 252 |
| ACGAACCT | GCCTCCCTCGCGCCATCAGACGAACCTCATGCT |
| SEQ ID NO: 253 | GCCTCCCGTAGGAGT |
| | SEQ ID NO: 254 |
| ACGAACGA | GCCTCCCTCGCGCCATCAGACGAACGACATGCT |
| SEQ ID NO: 255 | GCCTCCCGTAGGAGT |
| | SEQ ID NO: 256 |
| ACGAAGCA | GCCTCCCTCGCGCCATCAGACGAAGCACATGCT |
| SEQ ID NO: 257 | GCCTCCCGTAGGAGT |
| | SEQ ID NO: 258 |
| ACGAAGGT | GCCTCCCTCGCGCCATCAGACGAAGGTCATGCT |
| SEQ ID NO: 259 | GCCTCCCGTAGGAGT |
| | SEQ ID NO: 260 |
| ACGACAAG | GCCTCCCTCGCGCCATCAGACGACAAGCATGCT |
| SEQ ID NO: 261 | GCCTCCCGTAGGAGT |
| | SEQ ID NO: 262 |
| ACGACATC | GCCTCCCTCGCGCCATCAGACGACATCCATGCT |
| SEQ ID NO: 263 | GCCTCCCGTAGGAGT |
| | SEQ ID NO: 264 |
| ACGACTAC | GCCTCCCTCGCGCCATCAGACGACTACCATGCT |
| SEQ ID NO: 265 | GCCTCCCGTAGGAGT |
| | SEQ ID NO: 266 |
| ACGACTTG | GCCTCCCTCGCGCCATCAGACGACTTGCATGCT |
| SEQ ID NO: 267 | GCCTCCCGTAGGAGT |
| | SEQ ID NO: 268 |
| ACGAGAAC | GCCTCCCTCGCGCCATCAGACGAGAACCATGCT |
| SEQ ID NO: 269 | GCCTCCCGTAGGAGT |
| | SEQ ID NO: 270 |
| ACGAGATG | GCCTCCCTCGCGCCATCAGACGAGATGCATGCT |
| SEQ ID NO: 271 | GCCTCCCGTAGGAGT |
| | SEQ ID NO: 272 |
| ACGAGTAG | GCCTCCCTCGCGCCATCAGACGAGTAGCATGCT |
| SEQ ID NO: 273 | GCCTCCCGTAGGAGT |
| | SEQ ID NO: 274 |
| ACGAGTTC | GCCTCCCTCGCGCCATCAGACGAGTTCCATGCT |
| SEQ ID NO: 275 | GCCTCCCGTAGGAGT |
| | SEQ ID NO: 276 |
| ACGATCCA | GCCTCCCTCGCGCCATCAGACGATCCACATGCT |
| SEQ ID NO: 277 | GCCTCCCGTAGGAGT |
| | SEQ ID NO: 278 |
| ACGATCGT | GCCTCCCTCGCGCCATCAGACGATCGTCATGCT |
| SEQ ID NO: 279 | GCCTCCCGTAGGAGT |
| | SEQ ID NO: 280 |
| ACGATGCT | GCCTCCCTCGCGCCATCAGACGATGCTCATGCT |
| SEQ ID NO: 281 | GCCTCCCGTAGGAGT |
| | SEQ ID NO: 282 |
| ACGATGGA | GCCTCCCTCGCGCCATCAGACGATGGACATGCT |
| SEQ ID NO: 283 | GCCTCCCGTAGGAGT |
| | SEQ ID NO: 284 |
| ACGTACCA | GCCTCCCTCGCGCCATCAGACGTACCACATGCT |
| SEQ ID NO: 285 | GCCTCCCGTAGGAGT |
| | SEQ ID NO: 286 |
| ACGTACGT | GCCTCCCTCGCGCCATCAGACGTACGTCATGCT |
| SEQ ID NO: 287 | GCCTCCCGTAGGAGT |
| | SEQ ID NO: 288 |
| ACGTAGCT | GCCTCCCTCGCGCCATCAGACGTAGCTCATGCT |
| SEQ ID NO: 289 | GCCTCCCGTAGGAGT |
| | SEQ ID NO: 290 |
| ACGTAGGA | GCCTCCCTCGCGCCATCAGACGTAGGACATGCT |
| SEQ ID NO: 291 | GCCTCCCGTAGGAGT |
| | SEQ ID NO: 292 |
| ACGTCAAC | GCCTCCCTCGCGCCATCAGACGTCAACCATGCT |
| SEQ ID NO: 293 | GCCTCCCGTAGGAGT |
| | SEQ ID NO: 294 |
| ACGTCATG | GCCTCCCTCGCGCCATCAGACGTCATGCATGCT |
| SEQ ID NO: 295 | GCCTCCCGTAGGAGT |
| | SEQ ID NO: 296 |
| ACGTCTAG | GCCTCCCTCGCGCCATCAGACGTCTAGCATGCT |
| SEQ ID NO: 297 | GCCTCCCGTAGGAGT |
| | SEQ ID NO: 298 |
| ACGTCTTC | GCCTCCCTCGCGCCATCAGACGTCTTCCATGCT |
| SEQ ID NO: 299 | GCCTCCCGTAGGAGT |
| | SEQ ID NO: 300 |
| ACGTGAAG | GCCTCCCTCGCGCCATCAGACGTGAAGCATGCT |
| SEQ ID NO: 301 | GCCTCCCGTAGGAGT |
| | SEQ ID NO: 302 |
| ACGTGATC | GCCTCCCTCGCGCCATCAGACGTGATCCATGCT |
| SEQ ID NO: 303 | GCCTCCCGTAGGAGT |
| | SEQ ID NO: 304 |
| ACGTGTAC | GCCTCCCTCGCGCCATCAGACGTGTACCATGCT |
| SEQ ID NO: 305 | GCCTCCCGTAGGAGT |
| | SEQ ID NO: 306 |
| ACGTGTTG | GCCTCCCTCGCGCCATCAGACGTGTTGCATGCT |
| SEQ ID NO: 307 | GCCTCCCGTAGGAGT |
| | SEQ ID NO: 308 |
| ACGTTCCT | GCCTCCCTCGCGCCATCAGACGTTCCTCATGCT |
| SEQ ID NO: 309 | GCCTCCCGTAGGAGT |
| | SEQ ID NO: 310 |
| ACGTTCGA | GCCTCCCTCGCGCCATCAGACGTTCGACATGCT |
| SEQ ID NO: 311 | GCCTCCCGTAGGAGT |
| | SEQ ID NO: 312 |
| ACGTTGCA | GCCTCCCTCGCGCCATCAGACGTTGCACATGCT |
| SEQ ID NO: 313 | GCCTCCCGTAGGAGT |
| | SEQ ID NO: 314 |
| ACGTTGGT | GCCTCCCTCGCGCCATCAGACGTTGGTCATGCT |
| SEQ ID NO: 315 | GCCTCCCGTAGGAGT |
| | SEQ ID NO: 316 |
| ACTCACAG | GCCTCCCTCGCGCCATCAGACTCACAGCATGCT |
| SEQ ID NO: 317 | GCCTCCCGTAGGAGT |
| | SEQ ID NO: 318 |
| ACTCACTC | GCCTCCCTCGCGCCATCAGACTCACTCCATGCT |
| SEQ ID NO: 319 | GCCTCCCGTAGGAGT |
| | SEQ ID NO: 320 |
| ACTCAGAC | GCCTCCCTCGCGCCATCAGACTCAGACCATGCT |
| SEQ ID NO: 321 | GCCTCCCGTAGGAGT |
| | SEQ ID NO: 322 |
| ACTCAGTG | GCCTCCCTCGCGCCATCAGACTCAGTGCATGCT |
| SEQ ID NO: 323 | GCCTCCCGTAGGAGT |
| | SEQ ID NO: 324 |
| ACTCCACT | GCCTCCCTCGCGCCATCAGACTCCACTCATGCT |
| SEQ ID NO: 325 | GCCTCCCGTAGGAGT |
| | SEQ ID NO: 326 |
| ACTCCAGA | GCCTCCCTCGCGCCATCAGACTCCAGACATGCT |
| SEQ ID NO: 327 | GCCTCCCGTAGGAGT |
| | SEQ ID NO: 328 |
| ACTCCTCA | GCCTCCCTCGCGCCATCAGACTCCTCACATGCT |
| SEQ ID NO: 329 | GCCTCCCGTAGGAGT |
| | SEQ ID NO: 330 |
| ACTCCTGT | GCCTCCCTCGCGCCATCAGACTCCTGTCATGCT |
| SEQ ID NO: 331 | GCCTCCCGTAGGAGT |
| | SEQ ID NO: 332 |
| ACTCGACA | GCCTCCCTCGCGCCATCAGACTCGACACATGCT |
| SEQ ID NO: 333 | GCCTCCCGTAGGAGT |
| | SEQ ID NO: 334 |
| ACTCGAGT | GCCTCCCTCGCGCCATCAGACTCGAGTCATGCT |
| SEQ ID NO: 335 | GCCTCCCGTAGGAGT |
| | SEQ ID NO: 336 |
| ACTCGTCT | GCCTCCCTCGCGCCATCAGACTCGTCTCATGCT |
| SEQ ID NO: 337 | GCCTCCCGTAGGAGT |
| | SEQ ID NO: 338 |
| ACTCGTGA | GCCTCCCTCGCGCCATCAGACTCGTGACATGCT |
| SEQ ID NO: 339 | GCCTCCCGTAGGAGT |
| | SEQ ID NO: 340 |
| ACTCTCAC | GCCTCCCTCGCGCCATCAGACTCTCACCATGCT |
| SEQ ID NO: 341 | GCCTCCCGTAGGAGT |
| | SEQ ID NO: 342 |
| ACTCTCTG | GCCTCCCTCGCGCCATCAGACTCTCTGCATGCT |
| SEQ ID NO: 343 | GCCTCCCGTAGGAGT |
| | SEQ ID NO: 344 |
| ACTCTGAG | GCCTCCCTCGCGCCATCAGACTCTGAGCATGCT |
| SEQ ID NO: 345 | GCCTCCCGTAGGAGT |
| | SEQ ID NO: 346 |
| ACTCTGTC | GCCTCCCTCGCGCCATCAGACTCTGTCCATGCT |
| SEQ ID NO: 347 | GCCTCCCGTAGGAGT |
| | SEQ ID NO: 348 |
| ACTGACAC | GCCTCCCTCGCGCCATCAGACTGACACCATGCT |
| SEQ ID NO: 349 | GCCTCCCGTAGGAGT |
| | SEQ ID NO: 350 |
| ACTGACTG | GCCTCCCTCGCGCCATCAGACTGACTGCATGCT |
| SEQ ID NO: 351 | GCCTCCCGTAGGAGT |
| | SEQ ID NO: 352 |
| ACTGAGAG | GCCTCCCTCGCGCCATCAGACTGAGAGCATGCT |
| SEQ ID NO: 353 | GCCTCCCGTAGGAGT |
| | SEQ ID NO: 354 |
| ACTGAGTC | GCCTCCCTCGCGCCATCAGACTGAGTCCATGCT |
| SEQ ID NO: 355 | GCCTCCCGTAGGAGT |
| | SEQ ID NO: 356 |
| ACTGCACA | GCCTCCCTCGCGCCATCAGACTGCACACATGCT |
| SEQ ID NO: 357 | GCCTCCCGTAGGAGT |
| | SEQ ID NO: 358 |
| ACTGCAGT | GCCTCCCTCGCGCCATCAGACTGCAGTCATGCT |
| SEQ ID NO: 359 | GCCTCCCGTAGGAGT |
| | SEQ ID NO: 360 |
| ACTGCTCT | GCCTCCCTCGCGCCATCAGACTGCTCTCATGCT |
| SEQ ID NO: 361 | GCCTCCCGTAGGAGT |
| | SEQ ID NO: 362 |
| ACTGCTGA | GCCTCCCTCGCGCCATCAGACTGCTGACATGCT |
| SEQ ID NO: 363 | GCCTCCCGTAGGAGT |
| | SEQ ID NO: 364 |
| ACTGGACT | GCCTCCCTCGCGCCATCAGACTGGACTCATGCT |
| SEQ ID NO: 365 | GCCTCCCGTAGGAGT |
| | SEQ ID NO: 366 |
| ACTGGAGA | GCCTCCCTCGCGCCATCAGACTGGAGACATGCT |
| SEQ ID NO: 367 | GCCTCCCGTAGGAGT |
| | SEQ ID NO: 368 |
| ACTGGTCA | GCCTCCCTCGCGCCATCAGACTGGTCACATGCT |
| SEQ ID NO: 369 | GCCTCCCGTAGGAGT |
| | SEQ ID NO: 370 |
| ACTGGTGT | GCCTCCCTCGCGCCATCAGACTGGTGTCATGCT |
| SEQ ID NO: 371 | GCCTCCCGTAGGAGT |
| | SEQ ID NO: 372 |
| ACTGTCAG | GCCTCCCTCGCGCCATCAGACTGTCAGCATGCT |
| SEQ ID NO: 373 | GCCTCCCGTAGGAGT |
| | SEQ ID NO: 374 |
| ACTGTCTC | GCCTCCCTCGCGCCATCAGACTGTCTCCATGCT |
| SEQ ID NO: 375 | GCCTCCCGTAGGAGT |
| | SEQ ID NO: 376 |
| ACTGTGAC | GCCTCCCTCGCGCCATCAGACTGTGACCATGCT |
| SEQ ID NO: 377 | GCCTCCCGTAGGAGT |
| | SEQ ID NO: 378 |
| ACTGTGTG | GCCTCCCTCGCGCCATCAGACTGTGTGCATGCT |
| SEQ ID NO: 379 | GCCTCCCGTAGGAGT |
| | SEQ ID NO: 380 |
| AGACACAG | GCCTCCCTCGCGCCATCAGAGACACAGCATGCT |
| SEQ ID NO: 381 | GCCTCCCGTAGGAGT |
| | SEQ ID NO: 382 |
| AGACACTC | GCCTCCCTCGCGCCATCAGAGACACTCCATGCT |
| SEQ ID NO: 383 | GCCTCCCGTAGGAGT |
| | SEQ ID NO: 384 |
| AGACAGAC | GCCTCCCTCGCGCCATCAGAGACAGACCATGCT |
| SEQ ID NO: 385 | GCCTCCCGTAGGAGT |
| | SEQ ID NO: 386 |
| AGACAGTG | GCCTCCCTCGCGCCATCAGAGACAGTGCATGCT |
| SEQ ID NO: 387 | GCCTCCCGTAGGAGT |
| | SEQ ID NO: 388 |
| AGACCACT | GCCTCCCTCGCGCCATCAGAGACCACTCATGCT |
| SEQ ID NO: 389 | GCCTCCCGTAGGAGT |
| | SEQ ID NO: 390 |
| AGACCAGA | GCCTCCCTCGCGCCATCAGAGACCAGACATGCT |
| SEQ ID NO: 391 | GCCTCCCGTAGGAGT |
| | SEQ ID NO: 392 |
| AGACCTCA | GCCTCCCTCGCGCCATCAGAGACCTCACATGCT |
| SEQ ID NO: 393 | GCCTCCCGTAGGAGT |
| | SEQ ID NO: 394 |
| AGACCTGT | GCCTCCCTCGCGCCATCAGAGACCTGTCATGCT |
| SEQ ID NO: 395 | GCCTCCCGTAGGAGT |
| | SEQ ID NO: 396 |
| AGACGACA | GCCTCCCTCGCGCCATCAGAGACGACACATGCT |
| SEQ ID NO: 397 | GCCTCCCGTAGGAGT |
| | SEQ ID NO: 398 |
| AGACGAGT | GCCTCCCTCGCGCCATCAGAGACGAGTCATGCT |
| SEQ ID NO: 399 | GCCTCCCGTAGGAGT |
| | SEQ ID NO: 400 |
| AGACGTCT | GCCTCCCTCGCGCCATCAGAGACGTCTCATGCT |
| SEQ ID NO: 401 | GCCTCCCGTAGGAGT |
| | SEQ ID NO: 402 |
| AGACGTGA | GCCTCCCTCGCGCCATCAGAGACGTGACATGCT |
| SEQ ID NO: 403 | GCCTCCCGTAGGAGT |
| | SEQ ID NO: 404 |
| AGACTCAC | GCCTCCCTCGCGCCATCAGAGACTCACCATGCT |
| SEQ ID NO: 405 | GCCTCCCGTAGGAGT |
| | SEQ ID NO: 406 |
| AGACTCTG | GCCTCCCTCGCGCCATCAGAGACTCTGCATGCT |
| SEQ ID NO: 407 | GCCTCCCGTAGGAGT |
| | SEQ ID NO: 408 |
| AGACTGAG | GCCTCCCTCGCGCCATCAGAGACTGAGCATGCT |
| SEQ ID NO: 409 | GCCTCCCGTAGGAGT |
| | SEQ ID NO: 410 |
| AGACTGTC | GCCTCCCTCGCGCCATCAGAGACTGTCCATGCT |
| SEQ ID NO: 411 | GCCTCCCGTAGGAGT |
| | SEQ ID NO: 412 |
| AGAGACAC | GCCTCCCTCGCGCCATCAGAGAGACACCATGCT |
| SEQ ID NO: 413 | GCCTCCCGTAGGAGT |
| | SEQ ID NO: 414 |
| AGAGACTG | GCCTCCCTCGCGCCATCAGAGAGACTGCATGCT |
| SEQ ID NO: 415 | GCCTCCCGTAGGAGT |
| | SEQ ID NO: 416 |
| AGAGAGAG | GCCTCCCTCGCGCCATCAGAGAGAGAGCATGCT |
| SEQ ID NO: 417 | GCCTCCCGTAGGAGT |
| | SEQ ID NO: 418 |
| AGAGAGTC | GCCTCCCTCGCGCCATCAGAGAGAGTCCATGCT |
| SEQ ID NO: 419 | GCCTCCCGTAGGAGT |
| | SEQ ID NO: 420 |
| AGAGCACA | GCCTCCCTCGCGCCATCAGAGAGCACACATGCT |
| SEQ ID NO: 421 | GCCTCCCGTAGGAGT |
| | SEQ ID NO: 422 |
| AGAGCAGT | GCCTCCCTCGCGCCATCAGAGAGCAGTCATGCT |
| SEQ ID NO: 423 | GCCTCCCGTAGGAGT |
| | SEQ ID NO: 424 |
| AGAGCTCT | GCCTCCCTCGCGCCATCAGAGAGCTCTCATGCT |
| SEQ ID NO: 425 | GCCTCCCGTAGGAGT |
| | SEQ ID NO: 426 |
| AGAGCTGA | GCCTCCCTCGCGCCATCAGAGAGCTGACATGCT |
| SEQ ID NO: 427 | GCCTCCCGTAGGAGT |
| | SEQ ID NO: 428 |
| AGAGGACT | GCCTCCCTCGCGCCATCAGAGAGGACTCATGCT |
| SEQ ID NO: 429 | GCCTCCCGTAGGAGT |
| | SEQ ID NO: 430 |
| AGAGGAGA | GCCTCCCTCGCGCCATCAGAGAGGAGACATGCT |
| SEQ ID NO: 431 | GCCTCCCGTAGGAGT |
| | SEQ ID NO: 432 |
| AGAGGTCA | GCCTCCCTCGCGCCATCAGAGAGGTCACATGCT |
| SEQ ID NO: 433 | GCCTCCCGTAGGAGT |
| | SEQ ID NO: 434 |
| AGAGGTGT | GCCTCCCTCGCGCCATCAGAGAGGTGTCATGCT |
| SEQ ID NO: 435 | GCCTCCCGTAGGAGT |
| | SEQ ID NO: 436 |
| AGAGTCAG | GCCTCCCTCGCGCCATCAGAGAGTCAGCATGCT |
| SEQ ID NO: 437 | GCCTCCCGTAGGAGT |
| | SEQ ID NO: 438 |
| AGAGTCTC | GCCTCCCTCGCGCCATCAGAGAGTCTCCATGCT |
| SEQ ID NO: 439 | GCCTCCCGTAGGAGT |
| | SEQ ID NO: 440 |
| AGAGTGAC | GCCTCCCTCGCGCCATCAGAGAGTGACCATGCT |
| SEQ ID NO: 441 | GCCTCCCGTAGGAGT |
| | SEQ ID NO: 442 |
| AGAGTGTG | GCCTCCCTCGCGCCATCAGAGAGTGTGCATGCT |
| SEQ ID NO: 443 | GCCTCCCGTAGGAGT |
| | SEQ ID NO: 444 |
| AGCAACCT | GCCTCCCTCGCGCCATCAGAGCAACCTCATGCT |
| SEQ ID NO: 445 | GCCTCCCGTAGGAGT |
| | SEQ ID NO: 446 |
| AGCAACGA | GCCTCCCTCGCGCCATCAGAGCAACGACATGCT |
| SEQ ID NO: 447 | GCCTCCCGTAGGAGT |
| | SEQ ID NO: 448 |
| AGCAAGCA | GCCTCCCTCGCGCCATCAGAGCAAGCACATGCT |
| SEQ ID NO: 449 | GCCTCCCGTAGGAGT |
| | SEQ ID NO: 450 |
| AGCAAGGT | GCCTCCCTCGCGCCATCAGAGCAAGGTCATGCT |
| SEQ ID NO: 451 | GCCTCCCGTAGGAGT |
| | SEQ ID NO: 452 |
| AGCACAAG | GCCTCCCTCGCGCCATCAGAGCACAAGCATGCT |
| SEQ ID NO: 453 | GCCTCCCGTAGGAGT |
| | SEQ ID NO: 454 |
| AGCACATC | GCCTCCCTCGCGCCATCAGAGCACATCCATGCT |
| SEQ ID NO: 455 | GCCTCCCGTAGGAGT |
| | SEQ ID NO: 456 |
| AGCACTAC | GCCTCCCTCGCGCCATCAGAGCACTACCATGCT |
| SEQ ID NO: 457 | GCCTCCCGTAGGAGT |
| | SEQ ID NO: 458 |
| AGCACTTG | GCCTCCCTCGCGCCATCAGAGCACTTGCATGCT |
| SEQ ID NO: 459 | GCCTCCCGTAGGAGT |
| | SEQ ID NO: 460 |
| AGCAGAAC | GCCTCCCTCGCGCCATCAGAGCAGAACCATGCT |
| SEQ ID NO: 461 | GCCTCCCGTAGGAGT |
| | SEQ ID NO: 462 |
| AGCAGATG | GCCTCCCTCGCGCCATCAGAGCAGATGCATGCT |
| SEQ ID NO: 463 | GCCTCCCGTAGGAGT |
| | SEQ ID NO: 464 |
| AGCAGTAG | GCCTCCCTCGCGCCATCAGAGCAGTAGCATGCT |
| SEQ ID NO: 465 | GCCTCCCGTAGGAGT |
| | SEQ ID NO: 466 |
| AGCAGTTC | GCCTCCCTCGCGCCATCAGAGCAGTTCCATGCT |
| SEQ ID NO: 467 | GCCTCCCGTAGGAGT |
| | SEQ ID NO: 468 |
| AGCATCCA | GCCTCCCTCGCGCCATCAGAGCATCCACATGCT |
| SEQ ID NO: 469 | GCCTCCCGTAGGAGT |
| | SEQ ID NO: 470 |
| AGCATCGT | GCCTCCCTCGCGCCATCAGAGCATCGTCATGCT |
| SEQ ID NO: 471 | GCCTCCCGTAGGAGT |
| | SEQ ID NO: 472 |
| AGCATGCT | GCCTCCCTCGCGCCATCAGAGCATGCTCATGCT |
| SEQ ID NO: 473 | GCCTCCCGTAGGAGT |
| | SEQ ID NO: 474 |
| AGCATGGA | GCCTCCCTCGCGCCATCAGAGCATGGACATGCT |
| SEQ ID NO: 475 | GCCTCCCGTAGGAGT |
| | SEQ ID NO: 476 |
| AGCTACCA | GCCTCCCTCGCGCCATCAGAGCTACCACATGCT |
| SEQ ID NO: 477 | GCCTCCCGTAGGAGT |
| | SEQ ID NO: 478 |
| AGCTACGT | GCCTCCCTCGCGCCATCAGAGCTACGTCATGCT |
| SEQ ID NO: 479 | GCCTCCCGTAGGAGT |
| | SEQ ID NO: 480 |
| AGCTAGCT | GCCTCCCTCGCGCCATCAGAGCTAGCTCATGCT |
| SEQ ID NO: 481 | GCCTCCCGTAGGAGT |
| | SEQ ID NO: 482 |
| AGCTAGGA | GCCTCCCTCGCGCCATCAGAGCTAGGACATGCT |
| SEQ ID NO: 483 | GCCTCCCGTAGGAGT |
| | SEQ ID NO: 484 |
| AGCTCAAC | GCCTCCCTCGCGCCATCAGAGCTCAACCATGCT |
| SEQ ID NO: 485 | GCCTCCCGTAGGAGT |
| | SEQ ID NO: 486 |
| AGCTCATG | GCCTCCCTCGCGCCATCAGAGCTCATGCATGCT |
| SEQ ID NO: 487 | GCCTCCCGTAGGAGT |
| | SEQ ID NO: 488 |
| AGCTCTAG | GCCTCCCTCGCGCCATCAGAGCTCTAGCATGCT |
| SEQ ID NO: 489 | GCCTCCCGTAGGAGT |
| | SEQ ID NO: 490 |
| AGCTCTTC | GCCTCCCTCGCGCCATCAGAGCTCTTCCATGCT |
| SEQ ID NO: 491 | GCCTCCCGTAGGAGT |
| | SEQ ID NO: 492 |
| AGCTGAAG | GCCTCCCTCGCGCCATCAGAGCTGAAGCATGCT |
| SEQ ID NO: 493 | GCCTCCCGTAGGAGT |
| | SEQ ID NO: 494 |
| AGCTGATC | GCCTCCCTCGCGCCATCAGAGCTGATCCATGCT |
| SEQ ID NO: 495 | GCCTCCCGTAGGAGT |
| | SEQ ID NO: 496 |
| AGCTGTAC | GCCTCCCTCGCGCCATCAGAGCTGTACCATGCT |
| SEQ ID NO: 497 | GCCTCCCGTAGGAGT |
| | SEQ ID NO: 498 |
| AGCTGTTG | GCCTCCCTCGCGCCATCAGAGCTGTTGCATGCT |
| SEQ ID NO: 499 | GCCTCCCGTAGGAGT |
| | SEQ ID NO: 500 |
| AGCTTCCT | GCCTCCCTCGCGCCATCAGAGCTTCCTCATGCT |
| SEQ ID NO: 501 | GCCTCCCGTAGGAGT |
| | SEQ ID NO: 502 |
| AGCTTCGA | GCCTCCCTCGCGCCATCAGAGCTTCGACATGCT |
| SEQ ID NO: 503 | GCCTCCCGTAGGAGT |
| | SEQ ID NO: 504 |
| AGCTTGCA | GCCTCCCTCGCGCCATCAGAGCTTGCACATGCT |
| SEQ ID NO: 505 | GCCTCCCGTAGGAGT |
| | SEQ ID NO: 506 |
| AGCTTGGT | GCCTCCCTCGCGCCATCAGAGCTTGGTCATGCT |
| SEQ ID NO: 507 | GCCTCCCGTAGGAGT |
| | SEQ ID NO: 508 |
| AGGAACCA | GCCTCCCTCGCGCCATCAGAGGAACCACATGCT |
| SEQ ID NO: 509 | GCCTCCCGTAGGAGT |
| | SEQ ID NO: 510 |
| AGGAACGT | GCCTCCCTCGCGCCATCAGAGGAACGTCATGCT |
| SEQ ID NO: 511 | GCCTCCCGTAGGAGT |
| | SEQ ID NO: 512 |
| AGGAAGCT | GCCTCCCTCGCGCCATCAGAGGAAGCTCATGCT |
| SEQ ID NO: 513 | GCCTCCCGTAGGAGT |
| | SEQ ID NO: 514 |
| AGGAAGGA | GCCTCCCTCGCGCCATCAGAGGAAGGACATGCT |
| SEQ ID NO: 515 | GCCTCCCGTAGGAGT |
| | SEQ ID NO: 516 |
| AGGACAAC | GCCTCCCTCGCGCCATCAGAGGACAACCATGCT |
| SEQ ID NO: 517 | GCCTCCCGTAGGAGT |
| | SEQ ID NO: 518 |
| AGGACATG | GCCTCCCTCGCGCCATCAGAGGACATGCATGCT |
| SEQ ID NO: 519 | GCCTCCCGTAGGAGT |
| | SEQ ID NO: 520 |
| AGGACTAG | GCCTCCCTCGCGCCATCAGAGGACTAGCATGCT |
| SEQ ID NO: 521 | GCCTCCCGTAGGAGT |
| | SEQ ID NO: 522 |
| AGGACTTC | GCCTCCCTCGCGCCATCAGAGGACTTCCATGCT |
| SEQ ID NO: 523 | GCCTCCCGTAGGAGT |
| | SEQ ID NO: 524 |
| AGGAGAAG | GCCTCCCTCGCGCCATCAGAGGAGAAGCATGCT |
| SEQ ID NO: 525 | GCCTCCCGTAGGAGT |
| | SEQ ID NO: 526 |
| AGGAGATC | GCCTCCCTCGCGCCATCAGAGGAGATCCATGCT |
| SEQ ID NO: 527 | GCCTCCCGTAGGAGT |
| | SEQ ID NO: 528 |
| AGGAGTAC | GCCTCCCTCGCGCCATCAGAGGAGTACCATGCT |
| SEQ ID NO: 529 | GCCTCCCGTAGGAGT |
| | SEQ ID NO: 530 |
| AGGAGTTG | GCCTCCCTCGCGCCATCAGAGGAGTTGCATGCT |
| SEQ ID NO: 531 | GCCTCCCGTAGGAGT |
| | SEQ ID NO: 532 |
| AGGATCCT | GCCTCCCTCGCGCCATCAGAGGATCCTCATGCT |
| SEQ ID NO: 533 | GCCTCCCGTAGGAGT |
| | SEQ ID NO: 534 |
| AGGATCGA | GCCTCCCTCGCGCCATCAGAGGATCGACATGCT |
| SEQ ID NO: 535 | GCCTCCCGTAGGAGT |
| | SEQ ID NO: 536 |
| AGGATGCA | GCCTCCCTCGCGCCATCAGAGGATGCACATGCT |
| SEQ ID NO: 537 | GCCTCCCGTAGGAGT |
| | SEQ ID NO: 538 |
| AGGATGGT | GCCTCCCTCGCGCCATCAGAGGATGGTCATGCT |
| SEQ ID NO: 539 | GCCTCCCGTAGGAGT |
| | SEQ ID NO: 540 |
| AGGTACCT | GCCTCCCTCGCGCCATCAGAGGTACCTCATGCT |
| SEQ ID NO: 541 | GCCTCCCGTAGGAGT |
| | SEQ ID NO: 542 |
| AGGTACGA | GCCTCCCTCGCGCCATCAGAGGTACGACATGCT |
| SEQ ID NO: 543 | GCCTCCCGTAGGAGT |
| | SEQ ID NO: 544 |
| AGGTAGCA | GCCTCCCTCGCGCCATCAGAGGTAGCACATGCT |
| SEQ ID NO: 545 | GCCTCCCGTAGGAGT |
| | SEQ ID NO: 546 |
| AGGTAGGT | GCCTCCCTCGCGCCATCAGAGGTAGGTCATGCT |
| SEQ ID NO: 547 | GCCTCCCGTAGGAGT |
| | SEQ ID NO: 548 |
| AGGTCAAG | GCCTCCCTCGCGCCATCAGAGGTCAAGCATGCT |
| SEQ ID NO: 549 | GCCTCCCGTAGGAGT |
| | SEQ ID NO: 550 |
| AGGTCATC | GCCTCCCTCGCGCCATCAGAGGTCATCCATGCT |
| SEQ ID NO: 551 | GCCTCCCGTAGGAGT |
| | SEQ ID NO: 552 |
| AGGTCTAC | GCCTCCCTCGCGCCATCAGAGGTCTACCATGCT |
| SEQ ID NO: 553 | GCCTCCCGTAGGAGT |
| | SEQ ID NO: 554 |
| AGGTCTTG | GCCTCCCTCGCGCCATCAGAGGTCTTGCATGCT |
| SEQ ID NO: 555 | GCCTCCCGTAGGAGT |
| | SEQ ID NO: 556 |
| AGGTGAAC | GCCTCCCTCGCGCCATCAGAGGTGAACCATGCT |
| SEQ ID NO: 557 | GCCTCCCGTAGGAGT |
| | SEQ ID NO: 558 |
| AGGTGATG | GCCTCCCTCGCGCCATCAGAGGTGATGCATGCT |
| SEQ ID NO: 559 | GCCTCCCGTAGGAGT |
| | SEQ ID NO: 560 |
| AGGTGTAG | GCCTCCCTCGCGCCATCAGAGGTGTAGCATGCT |
| SEQ ID NO: 561 | GCCTCCCGTAGGAGT |
| | SEQ ID NO: 562 |
| AGGTGTTC | GCCTCCCTCGCGCCATCAGAGGTGTTCCATGCT |
| SEQ ID NO: 563 | GCCTCCCGTAGGAGT |
| | SEQ ID NO: 564 |
| AGGTTCCA | GCCTCCCTCGCGCCATCAGAGGTTCCACATGCT |
| SEQ ID NO: 565 | GCCTCCCGTAGGAGT |
| | SEQ ID NO: 566 |
| AGGTTCGT | GCCTCCCTCGCGCCATCAGAGGTTCGTCATGCT |
| SEQ ID NO: 567 | GCCTCCCGTAGGAGT |
| | SEQ ID NO: 568 |
| AGGTTGCT | GCCTCCCTCGCGCCATCAGAGGTTGCTCATGCT |
| SEQ ID NO: 569 | GCCTCCCGTAGGAGT |
| | SEQ ID NO: 570 |
| AGGTTGGA | GCCTCCCTCGCGCCATCAGAGGTTGGACATGCT |
| SEQ ID NO: 571 | GCCTCCCGTAGGAGT |
| | SEQ ID NO: 572 |
| AGTCACAC | GCCTCCCTCGCGCCATCAGAGTCACACCATGCT |
| SEQ ID NO: 573 | GCCTCCCGTAGGAGT |
| | SEQ ID NO: 574 |
| AGTCACTG | GCCTCCCTCGCGCCATCAGAGTCACTGCATGCT |
| SEQ ID NO: 575 | GCCTCCCGTAGGAGT |
| | SEQ ID NO: 576 |
| AGTCAGAG | GCCTCCCTCGCGCCATCAGAGTCAGAGCATGCT |
| SEQ ID NO: 577 | GCCTCCCGTAGGAGT |
| | SEQ ID NO: 578 |
| AGTCAGTC | GCCTCCCTCGCGCCATCAGAGTCAGTCCATGCT |
| SEQ ID NO: 579 | GCCTCCCGTAGGAGT |
| | SEQ ID NO: 580 |
| AGTCCACA | GCCTCCCTCGCGCCATCAGAGTCCACACATGCT |
| SEQ ID NO: 581 | GCCTCCCGTAGGAGT |
| | SEQ ID NO: 582 |
| AGTCCAGT | GCCTCCCTCGCGCCATCAGAGTCCAGTCATGCT |
| SEQ ID NO: 583 | GCCTCCCGTAGGAGT |
| | SEQ ID NO: 584 |
| AGTCCTCT | GCCTCCCTCGCGCCATCAGAGTCCTCTCATGCT |
| SEQ ID NO: 585 | GCCTCCCGTAGGAGT |
| | SEQ ID NO: 586 |
| AGTCCTGA | GCCTCCCTCGCGCCATCAGAGTCCTGACATGCT |
| SEQ ID NO: 587 | GCCTCCCGTAGGAGT |
| | SEQ ID NO: 588 |
| AGTCGACT | GCCTCCCTCGCGCCATCAGAGTCGACTCATGCT |
| SEQ ID NO: 589 | GCCTCCCGTAGGAGT |
| | SEQ ID NO: 590 |
| AGTCGAGA | GCCTCCCTCGCGCCATCAGAGTCGAGACATGCT |
| SEQ ID NO: 591 | GCCTCCCGTAGGAGT |
| | SEQ ID NO: 592 |
| AGTCGTCA | GCCTCCCTCGCGCCATCAGAGTCGTCACATGCT |
| SEQ ID NO: 593 | GCCTCCCGTAGGAGT |
| | SEQ ID NO: 594 |
| AGTCGTGT | GCCTCCCTCGCGCCATCAGAGTCGTGTCATGCT |
| SEQ ID NO: 595 | GCCTCCCGTAGGAGT |
| | SEQ ID NO: 596 |
| AGTCTCAG | GCCTCCCTCGCGCCATCAGAGTCTCAGCATGCT |
| SEQ ID NO: 597 | GCCTCCCGTAGGAGT |
| | SEQ ID NO: 598 |
| AGTCTCTC | GCCTCCCTCGCGCCATCAGAGTCTCTCCATGCT |
| SEQ ID NO: 599 | GCCTCCCGTAGGAGT |
| | SEQ ID NO: 600 |
| AGTCTGAC | GCCTCCCTCGCGCCATCAGAGTCTGACCATGCT |
| SEQ ID NO: 601 | GCCTCCCGTAGGAGT |
| | SEQ ID NO: 602 |
| AGTCTGTG | GCCTCCCTCGCGCCATCAGAGTCTGTGCATGCT |
| SEQ ID NO: 603 | GCCTCCCGTAGGAGT |
| | SEQ ID NO: 604 |
| AGTGACAG | GCCTCCCTCGCGCCATCAGAGTGACAGCATGCT |
| SEQ ID NO: 605 | GCCTCCCGTAGGAGT |
| | SEQ ID NO: 606 |
| AGTGACTC | GCCTCCCTCGCGCCATCAGAGTGACTCCATGCT |
| SEQ ID NO: 607 | GCCTCCCGTAGGAGT |
| | SEQ ID NO: 608 |
| AGTGAGAC | GCCTCCCTCGCGCCATCAGAGTGAGACCATGCT |
| SEQ ID NO: 609 | GCCTCCCGTAGGAGT |
| | SEQ ID NO: 610 |
| AGTGAGTG | GCCTCCCTCGCGCCATCAGAGTGAGTGCATGCT |
| SEQ ID NO: 611 | GCCTCCCGTAGGAGT |
| | SEQ ID NO: 612 |
| AGTGCACT | GCCTCCCTCGCGCCATCAGAGTGCACTCATGCT |
| SEQ ID NO: 613 | GCCTCCCGTAGGAGT |
| | SEQ ID NO: 614 |
| AGTGCAGA | GCCTCCCTCGCGCCATCAGAGTGCAGACATGCT |
| SEQ ID NO: 615 | GCCTCCCGTAGGAGT |
| | SEQ ID NO: 616 |
| AGTGCTCA | GCCTCCCTCGCGCCATCAGAGTGCTCACATGCT |
| SEQ ID NO: 617 | GCCTCCCGTAGGAGT |
| | SEQ ID NO: 618 |
| AGTGCTGT | GCCTCCCTCGCGCCATCAGAGTGCTGTCATGCT |
| SEQ ID NO: 619 | GCCTCCCGTAGGAGT |
| | SEQ ID NO: 620 |
| AGTGGACA | GCCTCCCTCGCGCCATCAGAGTGGACACATGCT |
| SEQ ID NO: 621 | GCCTCCCGTAGGAGT |
| | SEQ ID NO: 622 |
| AGTGGAGT | GCCTCCCTCGCGCCATCAGAGTGGAGTCATGCT |
| SEQ ID NO: 623 | GCCTCCCGTAGGAGT |
| | SEQ ID NO: 624 |
| AGTGGTCT | GCCTCCCTCGCGCCATCAGAGTGGTCTCATGCT |
| SEQ ID NO: 625 | GCCTCCCGTAGGAGT |
| | SEQ ID NO: 626 |
| AGTGGTGA | GCCTCCCTCGCGCCATCAGAGTGGTGACATGCT |
| SEQ ID NO: 627 | GCCTCCCGTAGGAGT |
| | SEQ ID NO: 628 |
| AGTGTCAC | GCCTCCCTCGCGCCATCAGAGTGTCACCATGCT |
| SEQ ID NO: 629 | GCCTCCCGTAGGAGT |
| | SEQ ID NO: 630 |
| AGTGTCTG | GCCTCCCTCGCGCCATCAGAGTGTCTGCATGCT |
| SEQ ID NO: 631 | GCCTCCCGTAGGAGT |
| | SEQ ID NO: 632 |
| AGTGTGAG | GCCTCCCTCGCGCCATCAGAGTGTGAGCATGCT |
| SEQ ID NO: 633 | GCCTCCCGTAGGAGT |
| | SEQ ID NO: 634 |
| AGTGTGTC | GCCTCCCTCGCGCCATCAGAGTGTGTCCATGCT |
| SEQ ID NO: 635 | GCCTCCCGTAGGAGT |
| | SEQ ID NO: 636 |
| ATAACCGC | GCCTCCCTCGCGCCATCAGATAACCGCCATGCT |
| SEQ ID NO: 637 | GCCTCCCGTAGGAGT |
| | SEQ ID NO: 638 |
| ATAACGCC | GCCTCCCTCGCGCCATCAGATAACGCCCATGCT |
| SEQ ID NO: 639 | GCCTCCCGTAGGAGT |
| | SEQ ID NO: 640 |
| ATAAGCGG | GCCTCCCTCGCGCCATCAGATAAGCGGCATGCT |
| SEQ ID NO: 641 | GCCTCCCGTAGGAGT |
| | SEQ ID NO: 642 |
| ATAAGGCG | GCCTCCCTCGCGCCATCAGATAAGGCGCATGCT |
| SEQ ID NO: 643 | GCCTCCCGTAGGAGT |
| | SEQ ID NO: 644 |
| ATATCCGG | GCCTCCCTCGCGCCATCAGATATCCGGCATGCT |
| SEQ ID NO: 645 | GCCTCCCGTAGGAGT |
| | SEQ ID NO: 646 |
| ATATCGCG | GCCTCCCTCGCGCCATCAGATATCGCGCATGCT |
| SEQ ID NO: 647 | GCCTCCCGTAGGAGT |
| | SEQ ID NO: 648 |
| ATATCGGC | GCCTCCCTCGCGCCATCAGATATCGGCCATGCT |
| SEQ ID NO: 649 | GCCTCCCGTAGGAGT |
| | SEQ ID NO: 650 |
| ATATGCCG | GCCTCCCTCGCGCCATCAGATATGCCGCATGCT |
| SEQ ID NO: 651 | GCCTCCCGTAGGAGT |
| | SEQ ID NO: 652 |
| ATATGCGC | GCCTCCCTCGCGCCATCAGATATGCGCCATGCT |
| SEQ ID NO: 653 | GCCTCCCGTAGGAGT |
| | SEQ ID NO: 654 |
| ATATGGCC | GCCTCCCTCGCGCCATCAGATATGGCCCATGCT |
| SEQ ID NO: 655 | GCCTCCCGTAGGAGT |
| | SEQ ID NO: 656 |
| ATCCAACG | GCCTCCCTCGCGCCATCAGATCCAACGCATGCT |
| SEQ ID NO: 657 | GCCTCCCGTAGGAGT |
| | SEQ ID NO: 658 |
| ATCCAAGC | GCCTCCCTCGCGCCATCAGATCCAAGCCATGCT |
| SEQ ID NO: 659 | GCCTCCCGTAGGAGT |
| | SEQ ID NO: 660 |
| ATCCATCC | GCCTCCCTCGCGCCATCAGATCCATCCCATGCT |
| SEQ ID NO: 661 | GCCTCCCGTAGGAGT |
| | SEQ ID NO: 662 |
| ATCCATGG | GCCTCCCTCGCGCCATCAGATCCATGGCATGCT |
| SEQ ID NO: 663 | GCCTCCCGTAGGAGT |
| | SEQ ID NO: 664 |
| ATCCGCAA | GCCTCCCTCGCGCCATCAGATCCGCAACATGCT |
| SEQ ID NO: 665 | GCCTCCCGTAGGAGT |
| | SEQ ID NO: 666 |
| ATCCGCTT | GCCTCCCTCGCGCCATCAGATCCGCTTCATGCT |
| SEQ ID NO: 667 | GCCTCCCGTAGGAGT |
| | SEQ ID NO: 668 |
| ATCCGGAT | GCCTCCCTCGCGCCATCAGATCCGGATCATGCT |
| SEQ ID NO: 669 | GCCTCCCGTAGGAGT |
| | SEQ ID NO: 670 |
| ATCCGGTA | GCCTCCCTCGCGCCATCAGATCCGGTACATGCT |
| SEQ ID NO: 671 | GCCTCCCGTAGGAGT |
| | SEQ ID NO: 672 |
| ATCCTACC | GCCTCCCTCGCGCCATCAGATCCTACCCATGCT |
| SEQ ID NO: 673 | GCCTCCCGTAGGAGT |
| | SEQ ID NO: 674 |
| ATCCTAGG | GCCTCCCTCGCGCCATCAGATCCTAGGCATGCT |
| SEQ ID NO: 675 | GCCTCCCGTAGGAGT |
| | SEQ ID NO: 676 |
| ATCCTTCG | GCCTCCCTCGCGCCATCAGATCCTTCGCATGCT |
| SEQ ID NO: 677 | GCCTCCCGTAGGAGT |
| | SEQ ID NO: 678 |
| ATCCTTGC | GCCTCCCTCGCGCCATCAGATCCTTGCCATGCT |
| SEQ ID NO: 679 | GCCTCCCGTAGGAGT |
| | SEQ ID NO: 680 |
| ATCGAACC | GCCTCCCTCGCGCCATCAGATCGAACCCATGCT |
| SEQ ID NO: 681 | GCCTCCCGTAGGAGT |
| | SEQ ID NO: 682 |
| ATCGAAGG | GCCTCCCTCGCGCCATCAGATCGAAGGCATGCT |
| SEQ ID NO: 683 | GCCTCCCGTAGGAGT |
| | SEQ ID NO: 684 |
| ATCGATCG | GCCTCCCTCGCGCCATCAGATCGATCGCATGCT |
| SEQ ID NO: 685 | GCCTCCCGTAGGAGT |
| | SEQ ID NO: 686 |
| ATCGATGC | GCCTCCCTCGCGCCATCAGATCGATGCCATGCT |
| SEQ ID NO: 687 | GCCTCCCGTAGGAGT |
| | SEQ ID NO: 688 |
| ATCGCCAA | GCCTCCCTCGCGCCATCAGATCGCCAACATGCT |
| SEQ ID NO: 689 | GCCTCCCGTAGGAGT |
| | SEQ ID NO: 690 |
| ATCGCCTT | GCCTCCCTCGCGCCATCAGATCGCCTTCATGCT |
| SEQ ID NO: 691 | GCCTCCCGTAGGAGT |
| | SEQ ID NO: 692 |
| ATCGCGAT | GCCTCCCTCGCGCCATCAGATCGCGATCATGCT |
| SEQ ID NO: 693 | GCCTCCCGTAGGAGT |
| | SEQ ID NO: 694 |
| ATCGCGTA | GCCTCCCTCGCGCCATCAGATCGCGTACATGCT |
| SEQ ID NO: 695 | GCCTCCCGTAGGAGT |
| | SEQ ID NO: 696 |
| ATCGGCAT | GCCTCCCTCGCGCCATCAGATCGGCATCATGCT |
| SEQ ID NO: 697 | GCCTCCCGTAGGAGT |
| | SEQ ID NO: 698 |
| ATCGGCTA | GCCTCCCTCGCGCCATCAGATCGGCTACATGCT |
| SEQ ID NO: 699 | GCCTCCCGTAGGAGT |
| | SEQ ID NO: 700 |
| ATCGTACG | GCCTCCCTCGCGCCATCAGATCGTACGCATGCT |
| SEQ ID NO: 701 | GCCTCCCGTAGGAGT |
| | SEQ ID NO: 702 |
| ATCGTAGC | GCCTCCCTCGCGCCATCAGATCGTAGCCATGCT |
| SEQ ID NO: 703 | GCCTCCCGTAGGAGT |
| | SEQ ID NO: 704 |
| ATCGTTCC | GCCTCCCTCGCGCCATCAGATCGTTCCCATGCT |
| SEQ ID NO: 705 | GCCTCCCGTAGGAGT |
| | SEQ ID NO: 706 |
| ATCGTTGG | GCCTCCCTCGCGCCATCAGATCGTTGGCATGCT |
| SEQ ID NO: 707 | GCCTCCCGTAGGAGT |
| | SEQ ID NO: 708 |
| ATGCAACC | GCCTCCCTCGCGCCATCAGATGCAACCCATGCT |
| SEQ ID NO: 709 | GCCTCCCGTAGGAGT |
| | SEQ ID NO: 710 |
| ATGCAAGG | GCCTCCCTCGCGCCATCAGATGCAAGGCATGCT |
| SEQ ID NO: 711 | GCCTCCCGTAGGAGT |
| | SEQ ID NO: 712 |
| ATGCATCG | GCCTCCCTCGCGCCATCAGATGCATCGCATGCT |
| SEQ ID NO: 713 | GCCTCCCGTAGGAGT |
| | SEQ ID NO: 714 |
| ATGCATGC | GCCTCCCTCGCGCCATCAGATGCATGCCATGCT |
| SEQ ID NO: 715 | GCCTCCCGTAGGAGT |
| | SEQ ID NO: 716 |
| ATGCCGAT | GCCTCCCTCGCGCCATCAGATGCCGATCATGCT |
| SEQ ID NO: 717 | GCCTCCCGTAGGAGT |
| | SEQ ID NO: 718 |
| ATGCCGTA | GCCTCCCTCGCGCCATCAGATGCCGTACATGCT |
| SEQ ID NO: 719 | GCCTCCCGTAGGAGT |
| | SEQ ID NO: 720 |
| ATGCGCAT | GCCTCCCTCGCGCCATCAGATGCGCATCATGCT |
| SEQ ID NO: 721 | GCCTCCCGTAGGAGT |
| | SEQ ID NO: 722 |
| ATGCGCTA | GCCTCCCTCGCGCCATCAGATGCGCTACATGCT |
| SEQ ID NO: 723 | GCCTCCCGTAGGAGT |
| | SEQ ID NO: 724 |
| ATGCGGAA | GCCTCCCTCGCGCCATCAGATGCGGAACATGCT |
| SEQ ID NO: 725 | GCCTCCCGTAGGAGT |
| | SEQ ID NO: 726 |
| ATGCGGTT | GCCTCCCTCGCGCCATCAGATGCGGTTCATGCT |
| SEQ ID NO: 727 | GCCTCCCGTAGGAGT |
| | SEQ ID NO: 728 |
| ATGCTACG | GCCTCCCTCGCGCCATCAGATGCTACGCATGCT |
| SEQ ID NO: 729 | GCCTCCCGTAGGAGT |
| | SEQ ID NO: 730 |
| ATGCTAGC | GCCTCCCTCGCGCCATCAGATGCTAGCCATGCT |
| SEQ ID NO: 731 | GCCTCCCGTAGGAGT |
| | SEQ ID NO: 732 |
| ATGCTTCC | GCCTCCCTCGCGCCATCAGATGCTTCCCATGCT |
| SEQ ID NO: 733 | GCCTCCCGTAGGAGT |
| | SEQ ID NO: 734 |
| ATGCTTGG | GCCTCCCTCGCGCCATCAGATGCTTGGCATGCT |
| SEQ ID NO: 735 | GCCTCCCGTAGGAGT |
| | SEQ ID NO: 736 |
| ATGGAACG | GCCTCCCTCGCGCCATCAGATGGAACGCATGCT |
| SEQ ID NO: 737 | GCCTCCCGTAGGAGT |
| | SEQ ID NO: 738 |
| ATGGAAGC | GCCTCCCTCGCGCCATCAGATGGAAGCCATGCT |
| SEQ ID NO: 739 | GCCTCCCGTAGGAGT |
| | SEQ ID NO: 740 |
| ATGGATCC | GCCTCCCTCGCGCCATCAGATGGATCCCATGCT |
| SEQ ID NO: 741 | GCCTCCCGTAGGAGT |
| | SEQ ID NO: 742 |
| ATGGATGG | GCCTCCCTCGCGCCATCAGATGGATGGCATGCT |
| SEQ ID NO: 743 | GCCTCCCGTAGGAGT |
| | SEQ ID NO: 744 |
| ATGGCCAT | GCCTCCCTCGCGCCATCAGATGGCCATCATGCT |
| SEQ ID NO: 745 | GCCTCCCGTAGGAGT |
| | SEQ ID NO: 746 |
| ATGGCCTA | GCCTCCCTCGCGCCATCAGATGGCCTACATGCT |
| SEQ ID NO: 747 | GCCTCCCGTAGGAGT |
| | SEQ ID NO: 748 |
| ATGGCGAA | GCCTCCCTCGCGCCATCAGATGGCGAACATGCT |
| SEQ ID NO: 749 | GCCTCCCGTAGGAGT |
| | SEQ ID NO: 750 |
| ATGGCGTT | GCCTCCCTCGCGCCATCAGATGGCGTTCATGCT |
| SEQ ID NO: 751 | GCCTCCCGTAGGAGT |
| | SEQ ID NO: 752 |
| ATGGTACC | GCCTCCCTCGCGCCATCAGATGGTACCCATGCT |
| SEQ ID NO: 753 | GCCTCCCGTAGGAGT |
| | SEQ ID NO: 754 |
| ATGGTAGG | GCCTCCCTCGCGCCATCAGATGGTAGGCATGCT |
| SEQ ID NO: 755 | GCCTCCCGTAGGAGT |
| | SEQ ID NO: 756 |
| ATGGTTCG | GCCTCCCTCGCGCCATCAGATGGTTCGCATGCT |
| SEQ ID NO: 757 | GCCTCCCGTAGGAGT |
| | SEQ ID NO: 758 |
| ATGGTTGC | GCCTCCCTCGCGCCATCAGATGGTTGCCATGCT |
| SEQ ID NO: 759 | GCCTCCCGTAGGAGT |
| | SEQ ID NO: 760 |
| ATTACCGG | GCCTCCCTCGCGCCATCAGATTACCGGCATGCT |
| SEQ ID NO: 761 | GCCTCCCGTAGGAGT |
| | SEQ ID NO: 762 |
| ATTACGCG | GCCTCCCTCGCGCCATCAGATTACGCGCATGCT |
| SEQ ID NO: 763 | GCCTCCCGTAGGAGT |
| | SEQ ID NO: 764 |
| ATTACGGC | GCCTCCCTCGCGCCATCAGATTACGGCCATGCT |
| SEQ ID NO: 765 | GCCTCCCGTAGGAGT |
| | SEQ ID NO: 766 |
| ATTAGCCG | GCCTCCCTCGCGCCATCAGATTAGCCGCATGCT |
| SEQ ID NO: 767 | GCCTCCCGTAGGAGT |
| | SEQ ID NO: 768 |
| ATTAGCGC | GCCTCCCTCGCGCCATCAGATTAGCGCCATGCT |
| SEQ ID NO: 769 | GCCTCCCGTAGGAGT |
| | SEQ ID NO: 770 |
| ATTAGGCC | GCCTCCCTCGCGCCATCAGATTAGGCCCATGCT |
| SEQ ID NO: 771 | GCCTCCCGTAGGAGT |
| | SEQ ID NO: 772 |
| CAACACCA | GCCTCCCTCGCGCCATCAGCAACACCACATGCT |
| SEQ ID NO: 773 | GCCTCCCGTAGGAGT |
| | SEQ ID NO: 774 |
| CAACACGT | GCCTCCCTCGCGCCATCAGCAACACGTCATGCT |
| SEQ ID NO: 775 | GCCTCCCGTAGGAGT |
| | SEQ ID NO: 776 |
| CAACAGCT | GCCTCCCTCGCGCCATCAGCAACAGCTCATGCT |
| SEQ ID NO: 777 | GCCTCCCGTAGGAGT |
| | SEQ ID NO: 778 |
| CAACAGGA | GCCTCCCTCGCGCCATCAGCAACAGGACATGCT |
| SEQ ID NO: 779 | GCCTCCCGTAGGAGT |
| | SEQ ID NO: 780 |
| CAACCAAC | GCCTCCCTCGCGCCATCAGCAACCAACCATGCT |
| SEQ ID NO: 781 | GCCTCCCGTAGGAGT |
| | SEQ ID NO: 782 |
| CAACCATG | GCCTCCCTCGCGCCATCAGCAACCATGCATGCT |
| SEQ ID NO: 783 | GCCTCCCGTAGGAGT |
| | SEQ ID NO: 784 |
| CAACCTAG | GCCTCCCTCGCGCCATCAGCAACCTAGCATGCT |
| SEQ ID NO: 785 | GCCTCCCGTAGGAGT |
| | SEQ ID NO: 786 |
| CAACCTTC | GCCTCCCTCGCGCCATCAGCAACCTTCCATGCT |
| SEQ ID NO: 787 | GCCTCCCGTAGGAGT |
| | SEQ ID NO: 788 |
| CAACGAAG | GCCTCCCTCGCGCCATCAGCAACGAAGCATGCT |
| SEQ ID NO: 789 | GCCTCCCGTAGGAGT |
| | SEQ ID NO: 790 |
| CAACGATC | GCCTCCCTCGCGCCATCAGCAACGATCCATGCT |
| SEQ ID NO: 791 | GCCTCCCGTAGGAGT |
| | SEQ ID NO: 792 |
| CAACGTAC | GCCTCCCTCGCGCCATCAGCAACGTACCATGCT |
| SEQ ID NO: 793 | GCCTCCCGTAGGAGT |
| | SEQ ID NO: 794 |
| CAACGTTG | GCCTCCCTCGCGCCATCAGCAACGTTGCATGCT |
| SEQ ID NO: 795 | GCCTCCCGTAGGAGT |
| | SEQ ID NO: 796 |
| CAACTCCT | GCCTCCCTCGCGCCATCAGCAACTCCTCATGCT |
| SEQ ID NO: 797 | GCCTCCCGTAGGAGT |
| | SEQ ID NO: 798 |
| CAACTCGA | GCCTCCCTCGCGCCATCAGCAACTCGACATGCT |
| SEQ ID NO: 799 | GCCTCCCGTAGGAGT |
| | SEQ ID NO: 800 |
| CAACTGCA | GCCTCCCTCGCGCCATCAGCAACTGCACATGCT |
| SEQ ID NO: 801 | GCCTCCCGTAGGAGT |
| | SEQ ID NO: 802 |
| CAACTGGT | GCCTCCCTCGCGCCATCAGCAACTGGTCATGCT |
| SEQ ID NO: 803 | GCCTCCCGTAGGAGT |
| | SEQ ID NO: 804 |
| CAAGACCT | GCCTCCCTCGCGCCATCAGCAAGACCTCATGCT |
| SEQ ID NO: 805 | GCCTCCCGTAGGAGT |
| | SEQ ID NO: 806 |
| CAAGACGA | GCCTCCCTCGCGCCATCAGCAAGACGACATGCT |
| SEQ ID NO: 807 | GCCTCCCGTAGGAGT |
| | SEQ ID NO: 808 |
| CAAGAGCA | GCCTCCCTCGCGCCATCAGCAAGAGCACATGCT |
| SEQ ID NO: 809 | GCCTCCCGTAGGAGT |
| | SEQ ID NO: 810 |
| CAAGAGGT | GCCTCCCTCGCGCCATCAGCAAGAGGTCATGCT |
| SEQ ID NO: 811 | GCCTCCCGTAGGAGT |
| | SEQ ID NO: 812 |
| CAAGCAAG | GCCTCCCTCGCGCCATCAGCAAGCAAGCATGCT |
| SEQ ID NO: 813 | GCCTCCCGTAGGAGT |
| | SEQ ID NO: 814 |
| CAAGCATC | GCCTCCCTCGCGCCATCAGCAAGCATCCATGCT |
| SEQ ID NO: 815 | GCCTCCCGTAGGAGT |
| | SEQ ID NO: 816 |
| CAAGCTAC | GCCTCCCTCGCGCCATCAGCAAGCTACCATGCT |
| SEQ ID NO: 817 | GCCTCCCGTAGGAGT |
| | SEQ ID NO: 818 |
| CAAGCTTG | GCCTCCCTCGCGCCATCAGCAAGCTTGCATGCT |
| SEQ ID NO: 819 | GCCTCCCGTAGGAGT |
| | SEQ ID NO: 820 |
| CAAGGAAC | GCCTCCCTCGCGCCATCAGCAAGGAACCATGCT |
| SEQ ID NO: 821 | GCCTCCCGTAGGAGT |
| | SEQ ID NO: 822 |
| CAAGGATG | GCCTCCCTCGCGCCATCAGCAAGGATGCATGCT |
| SEQ ID NO: 823 | GCCTCCCGTAGGAGT |
| | SEQ ID NO: 824 |
| CAAGGTAG | GCCTCCCTCGCGCCATCAGCAAGGTAGCATGCT |
| SEQ ID NO: 825 | GCCTCCCGTAGGAGT |
| | SEQ ID NO: 826 |
| CAAGGTTC | GCCTCCCTCGCGCCATCAGCAAGGTTCCATGCT |
| SEQ ID NO: 827 | GCCTCCCGTAGGAGT |
| | SEQ ID NO: 828 |
| CAAGTCCA | GCCTCCCTCGCGCCATCAGCAAGTCCACATGCT |
| SEQ ID NO: 829 | GCCTCCCGTAGGAGT |
| | SEQ ID NO: 830 |
| CAAGTCGT | GCCTCCCTCGCGCCATCAGCAAGTCGTCATGCT |
| SEQ ID NO: 831 | GCCTCCCGTAGGAGT |
| | SEQ ID NO: 832 |
| CAAGTGCT | GCCTCCCTCGCGCCATCAGCAAGTGCTCATGCT |
| SEQ ID NO: 833 | GCCTCCCGTAGGAGT |
| | SEQ ID NO: 834 |
| CAAGTGGA | GCCTCCCTCGCGCCATCAGCAAGTGGACATGCT |
| SEQ ID NO: 835 | GCCTCCCGTAGGAGT |
| | SEQ ID NO: 836 |
| CACAACAC | GCCTCCCTCGCGCCATCAGCACAACACCATGCT |
| SEQ ID NO: 837 | GCCTCCCGTAGGAGT |
| | SEQ ID NO: 838 |
| CACAACTG | GCCTCCCTCGCGCCATCAGCACAACTGCATGCT |
| SEQ ID NO: 839 | GCCTCCCGTAGGAGT |
| | SEQ ID NO: 840 |
| CACAAGAG | GCCTCCCTCGCGCCATCAGCACAAGAGCATGCT |
| SEQ ID NO: 841 | GCCTCCCGTAGGAGT |
| | SEQ ID NO: 842 |
| CACAAGTC | GCCTCCCTCGCGCCATCAGCACAAGTCCATGCT |
| SEQ ID NO: 843 | GCCTCCCGTAGGAGT |
| | SEQ ID NO: 844 |
| CACACACA | GCCTCCCTCGCGCCATCAGCACACACACATGCT |
| SEQ ID NO: 845 | GCCTCCCGTAGGAGT |
| | SEQ ID NO: 846 |
| CACACAGT | GCCTCCCTCGCGCCATCAGCACACAGTCATGCT |
| SEQ ID NO: 847 | GCCTCCCGTAGGAGT |
| | SEQ ID NO: 848 |
| CACACTCT | GCCTCCCTCGCGCCATCAGCACACTCTCATGCT |
| SEQ ID NO: 849 | GCCTCCCGTAGGAGT |
| | SEQ ID NO: 850 |
| CACACTGA | GCCTCCCTCGCGCCATCAGCACACTGACATGCT |
| SEQ ID NO: 851 | GCCTCCCGTAGGAGT |
| | SEQ ID NO: 852 |
| CACAGACT | GCCTCCCTCGCGCCATCAGCACAGACTCATGCT |
| SEQ ID NO: 853 | GCCTCCCGTAGGAGT |
| | SEQ ID NO: 854 |
| CACAGAGA | GCCTCCCTCGCGCCATCAGCACAGAGACATGCT |
| SEQ ID NO: 855 | GCCTCCCGTAGGAGT |
| | SEQ ID NO: 856 |
| CACAGTCA | GCCTCCCTCGCGCCATCAGCACAGTCACATGCT |
| SEQ ID NO: 857 | GCCTCCCGTAGGAGT |
| | SEQ ID NO: 858 |
| CACAGTGT | GCCTCCCTCGCGCCATCAGCACAGTGTCATGCT |
| SEQ ID NO: 859 | GCCTCCCGTAGGAGT |
| | SEQ ID NO: 860 |
| CACATCAG | GCCTCCCTCGCGCCATCAGCACATCAGCATGCT |
| SEQ ID NO: 861 | GCCTCCCGTAGGAGT |
| | SEQ ID NO: 862 |
| CACATCTC | GCCTCCCTCGCGCCATCAGCACATCTCCATGCT |
| SEQ ID NO: 863 | GCCTCCCGTAGGAGT |
| | SEQ ID NO: 864 |
| CACATGAC | GCCTCCCTCGCGCCATCAGCACATGACCATGCT |
| SEQ ID NO: 865 | GCCTCCCGTAGGAGT |
| | SEQ ID NO: 866 |
| CACATGTG | GCCTCCCTCGCGCCATCAGCACATGTGCATGCT |
| SEQ ID NO: 867 | GCCTCCCGTAGGAGT |
| | SEQ ID NO: 868 |
| CACTACAG | GCCTCCCTCGCGCCATCAGCACTACAGCATGCT |
| SEQ ID NO: 869 | GCCTCCCGTAGGAGT |
| | SEQ ID NO: 870 |
| CACTACTC | GCCTCCCTCGCGCCATCAGCACTACTCCATGCT |
| SEQ ID NO: 871 | GCCTCCCGTAGGAGT |
| | SEQ ID NO: 872 |
| CACTAGAC | GCCTCCCTCGCGCCATCAGCACTAGACCATGCT |
| SEQ ID NO: 873 | GCCTCCCGTAGGAGT |
| | SEQ ID NO: 874 |
| CACTAGTG | GCCTCCCTCGCGCCATCAGCACTAGTGCATGCT |
| SEQ ID NO: 875 | GCCTCCCGTAGGAGT |
| | SEQ ID NO: 876 |
| CACTCACT | GCCTCCCTCGCGCCATCAGCACTCACTCATGCT |
| SEQ ID NO: 877 | GCCTCCCGTAGGAGT |
| | SEQ ID NO: 878 |
| CACTCAGA | GCCTCCCTCGCGCCATCAGCACTCAGACATGCT |
| SEQ ID NO: 879 | GCCTCCCGTAGGAGT |
| | SEQ ID NO: 880 |
| CACTCTCA | GCCTCCCTCGCGCCATCAGCACTCTCACATGCT |
| SEQ ID NO: 881 | GCCTCCCGTAGGAGT |
| | SEQ ID NO: 882 |
| CACTCTGT | GCCTCCCTCGCGCCATCAGCACTCTGTCATGCT |
| SEQ ID NO: 883 | GCCTCCCGTAGGAGT |
| | SEQ ID NO: 884 |
| CACTGACA | GCCTCCCTCGCGCCATCAGCACTGACACATGCT |
| SEQ ID NO: 885 | GCCTCCCGTAGGAGT |
| | SEQ ID NO: 886 |
| CACTGAGT | GCCTCCCTCGCGCCATCAGCACTGAGTCATGCT |
| SEQ ID NO: 887 | GCCTCCCGTAGGAGT |
| | SEQ ID NO: 888 |
| CACTGTCT | GCCTCCCTCGCGCCATCAGCACTGTCTCATGCT |
| SEQ ID NO: 889 | GCCTCCCGTAGGAGT |
| | SEQ ID NO: 890 |
| CACTGTGA | GCCTCCCTCGCGCCATCAGCACTGTGACATGCT |
| SEQ ID NO: 891 | GCCTCCCGTAGGAGT |
| | SEQ ID NO: 892 |
| CACTTCAC | GCCTCCCTCGCGCCATCAGCACTTCACCATGCT |
| SEQ ID NO: 893 | GCCTCCCGTAGGAGT |
| | SEQ ID NO: 894 |
| CACTTCTG | GCCTCCCTCGCGCCATCAGCACTTCTGCATGCT |
| SEQ ID NO: 895 | GCCTCCCGTAGGAGT |
| | SEQ ID NO: 896 |
| CACTTGAG | GCCTCCCTCGCGCCATCAGCACTTGAGCATGCT |
| SEQ ID NO: 897 | GCCTCCCGTAGGAGT |
| | SEQ ID NO: 898 |
| CACTTGTC | GCCTCCCTCGCGCCATCAGCACTTGTCCATGCT |
| SEQ ID NO: 899 | GCCTCCCGTAGGAGT |
| | SEQ ID NO: 900 |
| CAGAACAG | GCCTCCCTCGCGCCATCAGCAGAACAGCATGCT |
| SEQ ID NO: 901 | GCCTCCCGTAGGAGT |
| | SEQ ID NO: 902 |
| CAGAACTC | GCCTCCCTCGCGCCATCAGCAGAACTCCATGCT |
| SEQ ID NO: 903 | GCCTCCCGTAGGAGT |
| | SEQ ID NO: 904 |
| CAGAAGAC | GCCTCCCTCGCGCCATCAGCAGAAGACCATGCT |
| SEQ ID NO: 905 | GCCTCCCGTAGGAGT |
| | SEQ ID NO: 906 |
| CAGAAGTG | GCCTCCCTCGCGCCATCAGCAGAAGTGCATGCT |
| SEQ ID NO: 907 | GCCTCCCGTAGGAGT |
| | SEQ ID NO: 908 |
| CAGACACT | GCCTCCCTCGCGCCATCAGCAGACACTCATGCT |
| SEQ ID NO: 909 | GCCTCCCGTAGGAGT |
| | SEQ ID NO: 910 |
| CAGACAGA | GCCTCCCTCGCGCCATCAGCAGACAGACATGCT |
| SEQ ID NO: 911 | GCCTCCCGTAGGAGT |
| | SEQ ID NO: 912 |
| CAGACTCA | GCCTCCCTCGCGCCATCAGCAGACTCACATGCT |
| SEQ ID NO: 913 | GCCTCCCGTAGGAGT |
| | SEQ ID NO: 914 |
| CAGACTGT | GCCTCCCTCGCGCCATCAGCAGACTGTCATGCT |
| SEQ ID NO: 915 | GCCTCCCGTAGGAGT |
| | SEQ ID NO: 916 |
| CAGAGACA | GCCTCCCTCGCGCCATCAGCAGAGACACATGCT |
| SEQ ID NO: 917 | GCCTCCCGTAGGAGT |
| | SEQ ID NO: 918 |
| CAGAGAGT | GCCTCCCTCGCGCCATCAGCAGAGAGTCATGCT |
| SEQ ID NO: 919 | GCCTCCCGTAGGAGT |
| | SEQ ID NO: 920 |
| CAGAGTCT | GCCTCCCTCGCGCCATCAGCAGAGTCTCATGCT |
| SEQ ID NO: 921 | GCCTCCCGTAGGAGT |
| | SEQ ID NO: 922 |
| CAGAGTGA | GCCTCCCTCGCGCCATCAGCAGAGTGACATGCT |
| SEQ ID NO: 923 | GCCTCCCGTAGGAGT |
| | SEQ ID NO: 924 |
| CAGATCAC | GCCTCCCTCGCGCCATCAGCAGATCACCATGCT |
| SEQ ID NO: 925 | GCCTCCCGTAGGAGT |
| | SEQ ID NO: 926 |
| CAGATCTG | GCCTCCCTCGCGCCATCAGCAGATCTGCATGCT |
| SEQ ID NO: 927 | GCCTCCCGTAGGAGT |
| | SEQ ID NO: 928 |
| CAGATGAG | GCCTCCCTCGCGCCATCAGCAGATGAGCATGCT |
| SEQ ID NO: 929 | GCCTCCCGTAGGAGT |
| | SEQ ID NO: 930 |
| CAGATGTC | GCCTCCCTCGCGCCATCAGCAGATGTCCATGCT |
| SEQ ID NO: 931 | GCCTCCCGTAGGAGT |
| | SEQ ID NO: 932 |
| CAGTACAC | GCCTCCCTCGCGCCATCAGCAGTACACCATGCT |
| SEQ ID NO: 933 | GCCTCCCGTAGGAGT |
| | SEQ ID NO: 934 |
| CAGTACTG | GCCTCCCTCGCGCCATCAGCAGTACTGCATGCT |
| SEQ ID NO: 935 | GCCTCCCGTAGGAGT |
| | SEQ ID NO: 936 |
| CAGTAGAG | GCCTCCCTCGCGCCATCAGCAGTAGAGCATGCT |
| SEQ ID NO: 937 | GCCTCCCGTAGGAGT |
| | SEQ ID NO: 938 |
| CAGTAGTC | GCCTCCCTCGCGCCATCAGCAGTAGTCCATGCT |
| SEQ ID NO: 939 | GCCTCCCGTAGGAGT |
| | SEQ ID NO: 940 |
| CAGTCACA | GCCTCCCTCGCGCCATCAGCAGTCACACATGCT |
| SEQ ID NO: 941 | GCCTCCCGTAGGAGT |
| | SEQ ID NO: 942 |
| CAGTCAGT | GCCTCCCTCGCGCCATCAGCAGTCAGTCATGCT |
| SEQ ID NO: 943 | GCCTCCCGTAGGAGT |
| | SEQ ID NO: 944 |
| CAGTCTCT | GCCTCCCTCGCGCCATCAGCAGTCTCTCATGCT |
| SEQ ID NO: 945 | GCCTCCCGTAGGAGT |
| | SEQ ID NO: 946 |
| CAGTCTGA | GCCTCCCTCGCGCCATCAGCAGTCTGACATGCT |
| SEQ ID NO: 947 | GCCTCCCGTAGGAGT |
| | SEQ ID NO: 948 |
| CAGTGACT | GCCTCCCTCGCGCCATCAGCAGTGACTCATGCT |
| SEQ ID NO: 949 | GCCTCCCGTAGGAGT |
| | SEQ ID NO: 950 |
| CAGTGAGA | GCCTCCCTCGCGCCATCAGCAGTGAGACATGCT |
| SEQ ID NO: 951 | GCCTCCCGTAGGAGT |
| | SEQ ID NO: 952 |
| CAGTGTCA | GCCTCCCTCGCGCCATCAGCAGTGTCACATGCT |
| SEQ ID NO: 953 | GCCTCCCGTAGGAGT |
| | SEQ ID NO: 954 |
| CAGTGTGT | GCCTCCCTCGCGCCATCAGCAGTGTGTCATGCT |
| SEQ ID NO: 955 | GCCTCCCGTAGGAGT |
| | SEQ ID NO: 956 |
| CAGTTCAG | GCCTCCCTCGCGCCATCAGCAGTTCAGCATGCT |
| SEQ ID NO: 957 | GCCTCCCGTAGGAGT |
| | SEQ ID NO: 958 |
| CAGTTCTC | GCCTCCCTCGCGCCATCAGCAGTTCTCCATGCT |
| SEQ ID NO: 959 | GCCTCCCGTAGGAGT |
| | SEQ ID NO: 960 |
| CAGTTGAC | GCCTCCCTCGCGCCATCAGCAGTTGACCATGCT |
| SEQ ID NO: 961 | GCCTCCCGTAGGAGT |
| | SEQ ID NO: 962 |
| CAGTTGTG | GCCTCCCTCGCGCCATCAGCAGTTGTGCATGCT |
| SEQ ID NO: 963 | GCCTCCCGTAGGAGT |
| | SEQ ID NO: 964 |
| CATCACCT | GCCTCCCTCGCGCCATCAGCATCACCTCATGCT |
| SEQ ID NO: 965 | GCCTCCCGTAGGAGT |
| | SEQ ID NO: 966 |
| CATCACGA | GCCTCCCTCGCGCCATCAGCATCACGACATGCT |
| SEQ ID NO: 967 | GCCTCCCGTAGGAGT |
| | SEQ ID NO: 968 |
| CATCAGCA | GCCTCCCTCGCGCCATCAGCATCAGCACATGCT |
| SEQ ID NO: 969 | GCCTCCCGTAGGAGT |
| | SEQ ID NO: 970 |
| CATCAGGT | GCCTCCCTCGCGCCATCAGCATCAGGTCATGCT |
| SEQ ID NO: 971 | GCCTCCCGTAGGAGT |
| | SEQ ID NO: 972 |
| CATCCAAG | GCCTCCCTCGCGCCATCAGCATCCAAGCATGCT |
| SEQ ID NO: 973 | GCCTCCCGTAGGAGT |
| | SEQ ID NO: 974 |
| CATCCATC | GCCTCCCTCGCGCCATCAGCATCCATCCATGCT |
| SEQ ID NO: 975 | GCCTCCCGTAGGAGT |
| | SEQ ID NO: 976 |
| CATCCTAC | GCCTCCCTCGCGCCATCAGCATCCTACCATGCT |
| SEQ ID NO: 977 | GCCTCCCGTAGGAGT |
| | SEQ ID NO: 978 |
| CATCCTTG | GCCTCCCTCGCGCCATCAGCATCCTTGCATGCT |
| SEQ ID NO: 979 | GCCTCCCGTAGGAGT |
| | SEQ ID NO: 980 |
| CATCGAAC | GCCTCCCTCGCGCCATCAGCATCGAACCATGCT |
| SEQ ID NO: 981 | GCCTCCCGTAGGAGT |
| | SEQ ID NO: 982 |
| CATCGATG | GCCTCCCTCGCGCCATCAGCATCGATGCATGCT |
| SEQ ID NO: 983 | GCCTCCCGTAGGAGT |
| | SEQ ID NO: 984 |
| CATCGTAG | GCCTCCCTCGCGCCATCAGCATCGTAGCATGCT |
| SEQ ID NO: 985 | GCCTCCCGTAGGAGT |
| | SEQ ID NO: 986 |
| CATCGTTC | GCCTCCCTCGCGCCATCAGCATCGTTCCATGCT |
| SEQ ID NO: 987 | GCCTCCCGTAGGAGT |
| | SEQ ID NO: 988 |
| CATCTCCA | GCCTCCCTCGCGCCATCAGCATCTCCACATGCT |
| SEQ ID NO: 989 | GCCTCCCGTAGGAGT |
| | SEQ ID NO: 990 |
| CATCTCGT | GCCTCCCTCGCGCCATCAGCATCTCGTCATGCT |
| SEQ ID NO: 991 | GCCTCCCGTAGGAGT |
| | SEQ ID NO: 992 |
| CATCTGCT | GCCTCCCTCGCGCCATCAGCATCTGCTCATGCT |
| SEQ ID NO: 993 | GCCTCCCGTAGGAGT |
| | SEQ ID NO: 994 |
| CATCTGGA | GCCTCCCTCGCGCCATCAGCATCTGGACATGCT |
| SEQ ID NO: 995 | GCCTCCCGTAGGAGT |
| | SEQ ID NO: 996 |
| CATGACCA | GCCTCCCTCGCGCCATCAGCATGACCACATGCT |
| SEQ ID NO: 997 | GCCTCCCGTAGGAGT |
| | SEQ ID NO: 998 |
| CATGACGT | GCCTCCCTCGCGCCATCAGCATGACGTCATGCT |
| SEQ ID NO: 999 | GCCTCCCGTAGGAGT |
| | SEQ ID NO: 1000 |
| CATGAGCT | GCCTCCCTCGCGCCATCAGCATGAGCTCATGCT |
| SEQ ID NO: 1001 | GCCTCCCGTAGGAGT |
| | SEQ ID NO: 1002 |
| CATGAGGA | GCCTCCCTCGCGCCATCAGCATGAGGACATGCT |
| SEQ ID NO: 1003 | GCCTCCCGTAGGAGT |
| | SEQ ID NO: 1004 |
| CATGCAAC | GCCTCCCTCGCGCCATCAGCATGCAACCATGCT |
| SEQ ID NO: 1005 | GCCTCCCGTAGGAGT |
| | SEQ ID NO: 1006 |
| CATGCATG | GCCTCCCTCGCGCCATCAGCATGCATGCATGCT |
| SEQ ID NO: 1007 | GCCTCCCGTAGGAGT |
| | SEQ ID NO: 1008 |
| CATGCTAG | GCCTCCCTCGCGCCATCAGCATGCTAGCATGCT |
| SEQ ID NO: 1009 | GCCTCCCGTAGGAGT |
| | SEQ ID NO: 1010 |
| CATGCTTC | GCCTCCCTCGCGCCATCAGCATGCTTCCATGCT |
| SEQ ID NO: 1011 | GCCTCCCGTAGGAGT |
| | SEQ ID NO: 1012 |
| CATGGAAG | GCCTCCCTCGCGCCATCAGCATGGAAGCATGCT |
| SEQ ID NO: 1013 | GCCTCCCGTAGGAGT |
| | SEQ ID NO: 1014 |
| CATGGATC | GCCTCCCTCGCGCCATCAGCATGGATCCATGCT |
| SEQ ID NO: 1015 | GCCTCCCGTAGGAGT |
| | SEQ ID NO: 1016 |
| CATGGTAC | GCCTCCCTCGCGCCATCAGCATGGTACCATGCT |
| SEQ ID NO: 1017 | GCCTCCCGTAGGAGT |
| | SEQ ID NO: 1018 |
| CATGGTTG | GCCTCCCTCGCGCCATCAGCATGGTTGCATGCT |
| SEQ ID NO: 1019 | GCCTCCCGTAGGAGT |
| | SEQ ID NO: 1020 |
| CATGTCCT | GCCTCCCTCGCGCCATCAGCATGTCCTCATGCT |
| SEQ ID NO: 1021 | GCCTCCCGTAGGAGT |
| | SEQ ID NO: 1022 |
| CATGTCGA | GCCTCCCTCGCGCCATCAGCATGTCGACATGCT |
| SEQ ID NO: 1023 | GCCTCCCGTAGGAGT |
| | SEQ ID NO: 1024 |
| CATGTGCA | GCCTCCCTCGCGCCATCAGCATGTGCACATGCT |
| SEQ ID NO: 1025 | GCCTCCCGTAGGAGT |
| | SEQ ID NO: 1026 |
| CATGTGGT | GCCTCCCTCGCGCCATCAGCATGTGGTCATGCT |
| SEQ ID NO: 1027 | GCCTCCCGTAGGAGT |
| | SEQ ID NO: 1028 |
| CCAACCAA | GCCTCCCTCGCGCCATCAGCCAACCAACATGCT |
| SEQ ID NO: 1029 | GCCTCCCGTAGGAGT |
| | SEQ ID NO: 1030 |
| CCAACCTT | GCCTCCCTCGCGCCATCAGCCAACCTTCATGCT |
| SEQ ID NO: 1031 | GCCTCCCGTAGGAGT |
| | SEQ ID NO: 1032 |
| CCAACGAT | GCCTCCCTCGCGCCATCAGCCAACGATCATGCT |
| SEQ ID NO: 1033 | GCCTCCCGTAGGAGT |
| | SEQ ID NO: 1034 |
| CCAACGTA | GCCTCCCTCGCGCCATCAGCCAACGTACATGCT |
| SEQ ID NO: 1035 | GCCTCCCGTAGGAGT |
| | SEQ ID NO: 1036 |
| CCAAGCAT | GCCTCCCTCGCGCCATCAGCCAAGCATCATGCT |
| SEQ ID NO: 1037 | GCCTCCCGTAGGAGT |
| | SEQ ID NO: 1038 |
| CCAAGCTA | GCCTCCCTCGCGCCATCAGCCAAGCTACATGCT |
| SEQ ID NO: 1039 | GCCTCCCGTAGGAGT |
| | SEQ ID NO: 1040 |
| CCAAGGAA | GCCTCCCTCGCGCCATCAGCCAAGGAACATGCT |
| SEQ ID NO: 1041 | GCCTCCCGTAGGAGT |
| | SEQ ID NO: 1042 |
| CCAAGGTT | GCCTCCCTCGCGCCATCAGCCAAGGTTCATGCT |
| SEQ ID NO: 1043 | GCCTCCCGTAGGAGT |
| | SEQ ID NO: 1044 |
| CCAATACG | GCCTCCCTCGCGCCATCAGCCAATACGCATGCT |
| SEQ ID NO: 1045 | GCCTCCCGTAGGAGT |
| | SEQ ID NO: 1046 |
| CCAATAGC | GCCTCCCTCGCGCCATCAGCCAATAGCCATGCT |
| SEQ ID NO: 1047 | GCCTCCCGTAGGAGT |
| | SEQ ID NO: 1048 |
| CCAATTCC | GCCTCCCTCGCGCCATCAGCCAATTCCCATGCT |
| SEQ ID NO: 1049 | GCCTCCCGTAGGAGT |
| | SEQ ID NO: 1050 |
| CCAATTGG | GCCTCCCTCGCGCCATCAGCCAATTGGCATGCT |
| SEQ ID NO: 1051 | GCCTCCCGTAGGAGT |
| | SEQ ID NO: 1052 |
| CCATAACG | GCCTCCCTCGCGCCATCAGCCATAACGCATGCT |
| SEQ ID NO: 1053 | GCCTCCCGTAGGAGT |
| | SEQ ID NO: 1054 |
| CCATAAGC | GCCTCCCTCGCGCCATCAGCCATAAGCCATGCT |
| SEQ ID NO: 1055 | GCCTCCCGTAGGAGT |
| | SEQ ID NO: 1056 |
| CCATATCC | GCCTCCCTCGCGCCATCAGCCATATCCCATGCT |
| SEQ ID NO: 1057 | GCCTCCCGTAGGAGT |
| | SEQ ID NO: 1058 |
| CCATATGG | GCCTCCCTCGCGCCATCAGCCATATGGCATGCT |
| SEQ ID NO: 1059 | GCCTCCCGTAGGAGT |
| | SEQ ID NO: 1060 |
| CCATCCAT | GCCTCCCTCGCGCCATCAGCCATCCATCATGCT |
| SEQ ID NO: 1061 | GCCTCCCGTAGGAGT |
| | SEQ ID NO: 1062 |
| CCATCCTA | GCCTCCCTCGCGCCATCAGCCATCCTACATGCT |
| SEQ ID NO: 1063 | GCCTCCCGTAGGAGT |
| | SEQ ID NO: 1064 |
| CCATCGAA | GCCTCCCTCGCGCCATCAGCCATCGAACATGCT |
| SEQ ID NO: 1065 | GCCTCCCGTAGGAGT |
| | SEQ ID NO: 1066 |
| CCATCGTT | GCCTCCCTCGCGCCATCAGCCATCGTTCATGCT |
| SEQ ID NO: 1067 | GCCTCCCGTAGGAGT |
| | SEQ ID NO: 1068 |
| CCATGCAA | GCCTCCCTCGCGCCATCAGCCATGCAACATGCT |
| SEQ ID NO: 1069 | GCCTCCCGTAGGAGT |
| | SEQ ID NO: 1070 |
| CCATGCTT | GCCTCCCTCGCGCCATCAGCCATGCTTCATGCT |
| SEQ ID NO: 1071 | GCCTCCCGTAGGAGT |
| | SEQ ID NO: 1072 |
| CCATGGAT | GCCTCCCTCGCGCCATCAGCCATGGATCATGCT |
| SEQ ID NO: 1073 | GCCTCCCGTAGGAGT |
| | SEQ ID NO: 1074 |
| CCATGGTA | GCCTCCCTCGCGCCATCAGCCATGGTACATGCT |
| SEQ ID NO: 1075 | GCCTCCCGTAGGAGT |
| | SEQ ID NO: 1076 |
| CCATTACC | GCCTCCCTCGCGCCATCAGCCATTACCCATGCT |
| SEQ ID NO: 1077 | GCCTCCCGTAGGAGT |
| | SEQ ID NO: 1078 |
| CCATTAGG | GCCTCCCTCGCGCCATCAGCCATTAGGCATGCT |
| SEQ ID NO: 1079 | GCCTCCCGTAGGAGT |
| | SEQ ID NO: 1080 |
| CCGCAATA | GCCTCCCTCGCGCCATCAGCCGCAATACATGCT |
| SEQ ID NO: 1081 | GCCTCCCGTAGGAGT |
| | SEQ ID NO: 1082 |
| CCGCATAA | GCCTCCCTCGCGCCATCAGCCGCATAACATGCT |
| SEQ ID NO: 1083 | GCCTCCCGTAGGAGT |
| | SEQ ID NO: 1084 |
| CCGCTATT | GCCTCCCTCGCGCCATCAGCCGCTATTCATGCT |
| SEQ ID NO: 1085 | GCCTCCCGTAGGAGT |
| | SEQ ID NO: 1086 |
| CCGCTTAT | GCCTCCCTCGCGCCATCAGCCGCTTATCATGCT |
| SEQ ID NO: 1087 | GCCTCCCGTAGGAGT |
| | SEQ ID NO: 1088 |
| CCGGAATT | GCCTCCCTCGCGCCATCAGCCGGAATTCATGCT |
| SEQ ID NO: 1089 | GCCTCCCGTAGGAGT |
| | SEQ ID NO: 1090 |
| CCGGATAT | GCCTCCCTCGCGCCATCAGCCGGATATCATGCT |
| SEQ ID NO: 1091 | GCCTCCCGTAGGAGT |
| | SEQ ID NO: 1092 |
| CCGGATTA | GCCTCCCTCGCGCCATCAGCCGGATTACATGCT |
| SEQ ID NO: 1093 | GCCTCCCGTAGGAGT |
| | SEQ ID NO: 1094 |
| CCGGTAAT | GCCTCCCTCGCGCCATCAGCCGGTAATCATGCT |
| SEQ ID NO: 1095 | GCCTCCCGTAGGAGT |
| | SEQ ID NO: 1096 |
| CCGGTATA | GCCTCCCTCGCGCCATCAGCCGGTATACATGCT |
| SEQ ID NO: 1097 | GCCTCCCGTAGGAGT |
| | SEQ ID NO: 1098 |
| CCGGTTAA | GCCTCCCTCGCGCCATCAGCCGGTTAACATGCT |
| SEQ ID NO: 1099 | GCCTCCCGTAGGAGT |
| | SEQ ID NO: 1100 |
| CCTAATCC | GCCTCCCTCGCGCCATCAGCCTAATCCCATGCT |
| SEQ ID NO: 1101 | GCCTCCCGTAGGAGT |
| | SEQ ID NO: 1102 |
| CCTAATGG | GCCTCCCTCGCGCCATCAGCCTAATGGCATGCT |
| SEQ ID NO: 1103 | GCCTCCCGTAGGAGT |
| | SEQ ID NO: 1104 |
| CCTACCAT | GCCTCCCTCGCGCCATCAGCCTACCATCATGCT |
| SEQ ID NO: 1105 | GCCTCCCGTAGGAGT |
| | SEQ ID NO: 1106 |
| CCTACCTA | GCCTCCCTCGCGCCATCAGCCTACCTACATGCT |
| SEQ ID NO: 1107 | GCCTCCCGTAGGAGT |
| | SEQ ID NO: 1108 |
| CCTACGAA | GCCTCCCTCGCGCCATCAGCCTACGAACATGCT |
| SEQ ID NO: 1109 | GCCTCCCGTAGGAGT |
| | SEQ ID NO: 1110 |
| CCTACGTT | GCCTCCCTCGCGCCATCAGCCTACGTTCATGCT |
| SEQ ID NO: 1111 | GCCTCCCGTAGGAGT |
| | SEQ ID NO: 1112 |
| CCTAGCAA | GCCTCCCTCGCGCCATCAGCCTAGCAACATGCT |
| SEQ ID NO: 1113 | GCCTCCCGTAGGAGT |
| | SEQ ID NO: 1114 |
| CCTAGCTT | GCCTCCCTCGCGCCATCAGCCTAGCTTCATGCT |
| SEQ ID NO: 1115 | GCCTCCCGTAGGAGT |
| | SEQ ID NO: 1116 |
| CCTAGGAT | GCCTCCCTCGCGCCATCAGCCTAGGATCATGCT |
| SEQ ID NO: 1117 | GCCTCCCGTAGGAGT |
| | SEQ ID NO: 1118 |
| CCTAGGTA | GCCTCCCTCGCGCCATCAGCCTAGGTACATGCT |
| SEQ ID NO: 1119 | GCCTCCCGTAGGAGT |
| | SEQ ID NO: 1120 |
| CCTATACC | GCCTCCCTCGCGCCATCAGCCTATACCCATGCT |
| SEQ ID NO: 1121 | GCCTCCCGTAGGAGT |
| | SEQ ID NO: 1122 |
| CCTATAGG | GCCTCCCTCGCGCCATCAGCCTATAGGCATGCT |
| SEQ ID NO: 1123 | GCCTCCCGTAGGAGT |
| | SEQ ID NO: 1124 |
| CCTATTCG | GCCTCCCTCGCGCCATCAGCCTATTCGCATGCT |
| SEQ ID NO: 1125 | GCCTCCCGTAGGAGT |
| | SEQ ID NO: 1126 |
| CCTATTGC | GCCTCCCTCGCGCCATCAGCCTATTGCCATGCT |
| SEQ ID NO: 1127 | GCCTCCCGTAGGAGT |
| | SEQ ID NO: 1128 |
| CCTTAACC | GCCTCCCTCGCGCCATCAGCCTTAACCCATGCT |
| SEQ ID NO: 1129 | GCCTCCCGTAGGAGT |
| | SEQ ID NO: 1130 |
| CCTTAAGG | GCCTCCCTCGCGCCATCAGCCTTAAGGCATGCT |
| SEQ ID NO: 1131 | GCCTCCCGTAGGAGT |
| | SEQ ID NO: 1132 |
| CCTTATCG | GCCTCCCTCGCGCCATCAGCCTTATCGCATGCT |
| SEQ ID NO: 1133 | GCCTCCCGTAGGAGT |
| | SEQ ID NO: 1134 |
| CCTTATGC | GCCTCCCTCGCGCCATCAGCCTTATGCCATGCT |
| SEQ ID NO: 1135 | GCCTCCCGTAGGAGT |
| | SEQ ID NO: 1136 |
| CCTTCCAA | GCCTCCCTCGCGCCATCAGCCTTCCAACATGCT |
| SEQ ID NO: 1137 | GCCTCCCGTAGGAGT |
| | SEQ ID NO: 1138 |
| CCTTCCTT | GCCTCCCTCGCGCCATCAGCCTTCCTTCATGCT |
| SEQ ID NO: 1139 | GCCTCCCGTAGGAGT |
| | SEQ ID NO: 1140 |
| CCTTCGAT | GCCTCCCTCGCGCCATCAGCCTTCGATCATGCT |
| SEQ ID NO: 1141 | GCCTCCCGTAGGAGT |
| | SEQ ID NO: 1142 |
| CCTTCGTA | GCCTCCCTCGCGCCATCAGCCTTCGTACATGCT |
| SEQ ID NO: 1143 | GCCTCCCGTAGGAGT |
| | SEQ ID NO: 1144 |
| CCTTGCAT | GCCTCCCTCGCGCCATCAGCCTTGCATCATGCT |
| SEQ ID NO: 1145 | GCCTCCCGTAGGAGT |
| | SEQ ID NO: 1146 |
| CCTTGCTA | GCCTCCCTCGCGCCATCAGCCTTGCTACATGCT |
| SEQ ID NO: 1147 | GCCTCCCGTAGGAGT |
| | SEQ ID NO: 1148 |
| CCTTGGAA | GCCTCCCTCGCGCCATCAGCCTTGGAACATGCT |
| SEQ ID NO: 1149 | GCCTCCCGTAGGAGT |
| | SEQ ID NO: 1150 |
| CCTTGGTT | GCCTCCCTCGCGCCATCAGCCTTGGTTCATGCT |
| SEQ ID NO: 1151 | GCCTCCCGTAGGAGT |
| | SEQ ID NO: 1152 |
| CGAACCAT | GCCTCCCTCGCGCCATCAGCGAACCATCATGCT |
| SEQ ID NO: 1153 | GCCTCCCGTAGGAGT |
| | SEQ ID NO: 1154 |
| CGAACCTA | GCCTCCCTCGCGCCATCAGCGAACCTACATGCT |
| SEQ ID NO: 1155 | GCCTCCCGTAGGAGT |
| | SEQ ID NO: 1156 |
| CGAACGAA | GCCTCCCTCGCGCCATCAGCGAACGAACATGCT |
| SEQ ID NO: 1157 | GCCTCCCGTAGGAGT |
| | SEQ ID NO: 1158 |
| CGAACGTT | GCCTCCCTCGCGCCATCAGCGAACGTTCATGCT |
| SEQ ID NO: 1159 | GCCTCCCGTAGGAGT |
| | SEQ ID NO: 1160 |
| CGAAGCAA | GCCTCCCTCGCGCCATCAGCGAAGCAACATGCT |
| SEQ ID NO: 1161 | GCCTCCCGTAGGAGT |
| | SEQ ID NO: 1162 |
| CGAAGCTT | GCCTCCCTCGCGCCATCAGCGAAGCTTCATGCT |
| SEQ ID NO: 1163 | GCCTCCCGTAGGAGT |
| | SEQ ID NO: 1164 |
| CGAAGGAT | GCCTCCCTCGCGCCATCAGCGAAGGATCATGCT |
| SEQ ID NO: 1165 | GCCTCCCGTAGGAGT |
| | SEQ ID NO: 1166 |
| CGAAGGTA | GCCTCCCTCGCGCCATCAGCGAAGGTACATGCT |
| SEQ ID NO: 1167 | GCCTCCCGTAGGAGT |
| | SEQ ID NO: 1168 |
| CGAATACC | GCCTCCCTCGCGCCATCAGCGAATACCCATGCT |
| SEQ ID NO: 1169 | GCCTCCCGTAGGAGT |
| | SEQ ID NO: 1170 |
| CGAATAGG | GCCTCCCTCGCGCCATCAGCGAATAGGCATGCT |
| SEQ ID NO: 1171 | GCCTCCCGTAGGAGT |
| | SEQ ID NO: 1172 |
| CGAATTCG | GCCTCCCTCGCGCCATCAGCGAATTCGCATGCT |
| SEQ ID NO: 1173 | GCCTCCCGTAGGAGT |
| | SEQ ID NO: 1174 |
| CGAATTGC | GCCTCCCTCGCGCCATCAGCGAATTGCCATGCT |
| SEQ ID NO: 1175 | GCCTCCCGTAGGAGT |
| | SEQ ID NO: 1176 |
| CGATAACC | GCCTCCCTCGCGCCATCAGCGATAACCCATGCT |
| SEQ ID NO: 1177 | GCCTCCCGTAGGAGT |
| | SEQ ID NO: 1178 |
| CGATAAGG | GCCTCCCTCGCGCCATCAGCGATAAGGCATGCT |
| SEQ ID NO: 1179 | GCCTCCCGTAGGAGT |
| | SEQ ID NO: 1180 |
| CGATATCG | GCCTCCCTCGCGCCATCAGCGATATCGCATGCT |
| SEQ ID NO: 1181 | GCCTCCCGTAGGAGT |
| | SEQ ID NO: 1182 |
| CGATATGC | GCCTCCCTCGCGCCATCAGCGATATGCCATGCT |
| SEQ ID NO: 1183 | GCCTCCCGTAGGAGT |
| | SEQ ID NO: 1184 |
| CGATCCAA | GCCTCCCTCGCGCCATCAGCGATCCAACATGCT |
| SEQ ID NO: 1185 | GCCTCCCGTAGGAGT |
| | SEQ ID NO: 1186 |
| CGATCCTT | GCCTCCCTCGCGCCATCAGCGATCCTTCATGCT |
| SEQ ID NO: 1187 | GCCTCCCGTAGGAGT |
| | SEQ ID NO: 1188 |
| CGATCGAT | GCCTCCCTCGCGCCATCAGCGATCGATCATGCT |
| SEQ ID NO: 1189 | GCCTCCCGTAGGAGT |
| | SEQ ID NO: 1190 |
| CGATCGTA | GCCTCCCTCGCGCCATCAGCGATCGTACATGCT |
| SEQ ID NO: 1191 | GCCTCCCGTAGGAGT |
| | SEQ ID NO: 1192 |
| CGATGCAT | GCCTCCCTCGCGCCATCAGCGATGCATCATGCT |
| SEQ ID NO: 1193 | GCCTCCCGTAGGAGT |
| | SEQ ID NO: 1194 |
| CGATGCTA | GCCTCCCTCGCGCCATCAGCGATGCTACATGCT |
| SEQ ID NO: 1195 | GCCTCCCGTAGGAGT |
| | SEQ ID NO: 1196 |
| CGATGGAA | GCCTCCCTCGCGCCATCAGCGATGGAACATGCT |
| SEQ ID NO: 1197 | GCCTCCCGTAGGAGT |
| | SEQ ID NO: 1198 |
| CGATGGTT | GCCTCCCTCGCGCCATCAGCGATGGTTCATGCT |
| SEQ ID NO: 1199 | GCCTCCCGTAGGAGT |
| | SEQ ID NO: 1200 |
| CGATTACG | GCCTCCCTCGCGCCATCAGCGATTACGCATGCT |
| SEQ ID NO: 1201 | GCCTCCCGTAGGAGT |
| | SEQ ID NO: 1202 |
| CGATTAGC | GCCTCCCTCGCGCCATCAGCGATTAGCCATGCT |
| SEQ ID NO: 1203 | GCCTCCCGTAGGAGT |
| | SEQ ID NO: 1204 |
| CGCCAATA | GCCTCCCTCGCGCCATCAGCGCCAATACATGCT |
| SEQ ID NO: 1205 | GCCTCCCGTAGGAGT |
| | SEQ ID NO: 1206 |
| CGCCATAA | GCCTCCCTCGCGCCATCAGCGCCATAACATGCT |
| SEQ ID NO: 1207 | GCCTCCCGTAGGAGT |
| | SEQ ID NO: 1208 |
| CGCCTATT | GCCTCCCTCGCGCCATCAGCGCCTATTCATGCT |
| SEQ ID NO: 1209 | GCCTCCCGTAGGAGT |
| | SEQ ID NO: 1210 |
| CGCCTTAT | GCCTCCCTCGCGCCATCAGCGCCTTATCATGCT |
| SEQ ID NO: 1211 | GCCTCCCGTAGGAGT |
| | SEQ ID NO: 1212 |
| CGCGAATT | GCCTCCCTCGCGCCATCAGCGCGAATTCATGCT |
| SEQ ID NO: 1213 | GCCTCCCGTAGGAGT |
| | SEQ ID NO: 1214 |
| CGCGATAT | GCCTCCCTCGCGCCATCAGCGCGATATCATGCT |
| SEQ ID NO: 1215 | GCCTCCCGTAGGAGT |
| | SEQ ID NO: 1216 |
| CGCGATTA | GCCTCCCTCGCGCCATCAGCGCGATTACATGCT |
| SEQ ID NO: 1217 | GCCTCCCGTAGGAGT |
| | SEQ ID NO: 1218 |
| CGCGTAAT | GCCTCCCTCGCGCCATCAGCGCGTAATCATGCT |
| SEQ ID NO: 1219 | GCCTCCCGTAGGAGT |
| | SEQ ID NO: 1220 |
| CGCGTATA | GCCTCCCTCGCGCCATCAGCGCGTATACATGCT |
| SEQ ID NO: 1221 | GCCTCCCGTAGGAGT |
| | SEQ ID NO: 1222 |
| CGCGTTAA | GCCTCCCTCGCGCCATCAGCGCGTTAACATGCT |
| SEQ ID NO: 1223 | GCCTCCCGTAGGAGT |
| | SEQ ID NO: 1224 |
| CGGCAATT | GCCTCCCTCGCGCCATCAGCGGCAATTCATGCT |
| SEQ ID NO: 1225 | GCCTCCCGTAGGAGT |
| | SEQ ID NO: 1226 |
| CGGCATAT | GCCTCCCTCGCGCCATCAGCGGCATATCATGCT |
| SEQ ID NO: 1227 | GCCTCCCGTAGGAGT |
| | SEQ ID NO: 1228 |
| CGGCATTA | GCCTCCCTCGCGCCATCAGCGGCATTACATGCT |
| SEQ ID NO: 1229 | GCCTCCCGTAGGAGT |
| | SEQ ID NO: 1230 |
| CGGCTAAT | GCCTCCCTCGCGCCATCAGCGGCTAATCATGCT |
| SEQ ID NO: 1231 | GCCTCCCGTAGGAGT |
| | SEQ ID NO: 1232 |
| CGGCTATA | GCCTCCCTCGCGCCATCAGCGGCTATACATGCT |
| SEQ ID NO: 1233 | GCCTCCCGTAGGAGT |
| | SEQ ID NO: 1234 |
| CGGCTTAA | GCCTCCCTCGCGCCATCAGCGGCTTAACATGCT |
| SEQ ID NO: 1235 | GCCTCCCGTAGGAGT |
| | SEQ ID NO: 1236 |
| CGTAATCG | GCCTCCCTCGCGCCATCAGCGTAATCGCATGCT |
| SEQ ID NO: 1237 | GCCTCCCGTAGGAGT |
| | SEQ ID NO: 1238 |
| CGTAATGC | GCCTCCCTCGCGCCATCAGCGTAATGCCATGCT |
| SEQ ID NO: 1239 | GCCTCCCGTAGGAGT |
| | SEQ ID NO: 1240 |
| CGTACCAA | GCCTCCCTCGCGCCATCAGCGTACCAACATGCT |
| SEQ ID NO: 1241 | GCCTCCCGTAGGAGT |
| | SEQ ID NO: 1242 |
| CGTACCTT | GCCTCCCTCGCGCCATCAGCGTACCTTCATGCT |
| SEQ ID NO: 1243 | GCCTCCCGTAGGAGT |
| | SEQ ID NO: 1244 |
| CGTACGAT | GCCTCCCTCGCGCCATCAGCGTACGATCATGCT |
| SEQ ID NO: 1245 | GCCTCCCGTAGGAGT |
| | SEQ ID NO: 1246 |
| CGTACGTA | GCCTCCCTCGCGCCATCAGCGTACGTACATGCT |
| SEQ ID NO: 1247 | GCCTCCCGTAGGAGT |
| | SEQ ID NO: 1248 |
| CGTAGCAT | GCCTCCCTCGCGCCATCAGCGTAGCATCATGCT |
| SEQ ID NO: 1249 | GCCTCCCGTAGGAGT |
| | SEQ ID NO: 1250 |
| CGTAGCTA | GCCTCCCTCGCGCCATCAGCGTAGCTACATGCT |
| SEQ ID NO: 1251 | GCCTCCCGTAGGAGT |
| | SEQ ID NO: 1252 |
| CGTAGGAA | GCCTCCCTCGCGCCATCAGCGTAGGAACATGCT |
| SEQ ID NO: 1253 | GCCTCCCGTAGGAGT |
| | SEQ ID NO: 1254 |
| CGTAGGTT | GCCTCCCTCGCGCCATCAGCGTAGGTTCATGCT |
| SEQ ID NO: 1255 | GCCTCCCGTAGGAGT |
| | SEQ ID NO: 1256 |
| CGTATACG | GCCTCCCTCGCGCCATCAGCGTATACGCATGCT |
| SEQ ID NO: 1257 | GCCTCCCGTAGGAGT |
| | SEQ ID NO: 1258 |
| CGTATAGC | GCCTCCCTCGCGCCATCAGCGTATAGCCATGCT |
| SEQ ID NO: 1259 | GCCTCCCGTAGGAGT |
| | SEQ ID NO: 1260 |
| CGTATTCC | GCCTCCCTCGCGCCATCAGCGTATTCCCATGCT |
| SEQ ID NO: 1261 | GCCTCCCGTAGGAGT |
| | SEQ ID NO: 1262 |
| CGTATTGG | GCCTCCCTCGCGCCATCAGCGTATTGGCATGCT |
| SEQ ID NO: 1263 | GCCTCCCGTAGGAGT |
| | SEQ ID NO: 1264 |
| CGTTAACG | GCCTCCCTCGCGCCATCAGCGTTAACGCATGCT |
| SEQ ID NO: 1265 | GCCTCCCGTAGGAGT |
| | SEQ ID NO: 1266 |
| CGTTAAGC | GCCTCCCTCGCGCCATCAGCGTTAAGCCATGCT |
| SEQ ID NO: 1267 | GCCTCCCGTAGGAGT |
| | SEQ ID NO: 1268 |
| CGTTATCC | GCCTCCCTCGCGCCATCAGCGTTATCCCATGCT |
| SEQ ID NO: 1269 | GCCTCCCGTAGGAGT |
| | SEQ ID NO: 1270 |
| CGTTATGG | GCCTCCCTCGCGCCATCAGCGTTATGGCATGCT |
| SEQ ID NO: 1271 | GCCTCCCGTAGGAGT |
| | SEQ ID NO: 1272 |
| CGTTCCAT | GCCTCCCTCGCGCCATCAGCGTTCCATCATGCT |
| SEQ ID NO: 1273 | GCCTCCCGTAGGAGT |
| | SEQ ID NO: 1274 |
| CGTTCCTA | GCCTCCCTCGCGCCATCAGCGTTCCTACATGCT |
| SEQ ID NO: 1275 | GCCTCCCGTAGGAGT |
| | SEQ ID NO: 1276 |
| CGTTCGAA | GCCTCCCTCGCGCCATCAGCGTTCGAACATGCT |
| SEQ ID NO: 1277 | GCCTCCCGTAGGAGT |
| | SEQ ID NO: 1278 |
| CGTTCGTT | GCCTCCCTCGCGCCATCAGCGTTCGTTCATGCT |
| SEQ ID NO: 1279 | GCCTCCCGTAGGAGT |
| | SEQ ID NO: 1280 |
| CGTTGCAA | GCCTCCCTCGCGCCATCAGCGTTGCAACATGCT |
| SEQ ID NO: 1281 | GCCTCCCGTAGGAGT |
| | SEQ ID NO: 1282 |
| CGTTGCTT | GCCTCCCTCGCGCCATCAGCGTTGCTTCATGCT |
| SEQ ID NO: 1283 | GCCTCCCGTAGGAGT |
| | SEQ ID NO: 1284 |
| CGTTGGAT | GCCTCCCTCGCGCCATCAGCGTTGGATCATGCT |
| SEQ ID NO: 1285 | GCCTCCCGTAGGAGT |
| | SEQ ID NO: 1286 |
| CGTTGGTA | GCCTCCCTCGCGCCATCAGCGTTGGTACATGCT |
| SEQ ID NO: 1287 | GCCTCCCGTAGGAGT |
| | SEQ ID NO: 1288 |
| CTACACCT | GCCTCCCTCGCGCCATCAGCTACACCTCATGCT |
| SEQ ID NO: 1289 | GCCTCCCGTAGGAGT |
| | SEQ ID NO: 1290 |
| CTACACGA | GCCTCCCTCGCGCCATCAGCTACACGACATGCT |
| SEQ ID NO: 1291 | GCCTCCCGTAGGAGT |
| | SEQ ID NO: 1292 |
| CTACAGCA | GCCTCCCTCGCGCCATCAGCTACAGCACATGCT |
| SEQ ID NO: 1293 | GCCTCCCGTAGGAGT |
| | SEQ ID NO: 1294 |
| CTACAGGT | GCCTCCCTCGCGCCATCAGCTACAGGTCATGCT |
| SEQ ID NO: 1295 | GCCTCCCGTAGGAGT |
| | SEQ ID NO: 1296 |
| CTACCAAG | GCCTCCCTCGCGCCATCAGCTACCAAGCATGCT |
| SEQ ID NO: 1297 | GCCTCCCGTAGGAGT |
| | SEQ ID NO: 1298 |
| CTACCATC | GCCTCCCTCGCGCCATCAGCTACCATCCATGCT |
| SEQ ID NO: 1299 | GCCTCCCGTAGGAGT |
| | SEQ ID NO: 1300 |
| CTACCTAC | GCCTCCCTCGCGCCATCAGCTACCTACCATGCT |
| SEQ ID NO: 1301 | GCCTCCCGTAGGAGT |
| | SEQ ID NO: 1302 |
| CTACCTTG | GCCTCCCTCGCGCCATCAGCTACCTTGCATGCT |
| SEQ ID NO: 1303 | GCCTCCCGTAGGAGT |
| | SEQ ID NO: 1304 |
| CTACGAAC | GCCTCCCTCGCGCCATCAGCTACGAACCATGCT |
| SEQ ID NO: 1305 | GCCTCCCGTAGGAGT |
| | SEQ ID NO: 1306 |
| CTACGATG | GCCTCCCTCGCGCCATCAGCTACGATGCATGCT |
| SEQ ID NO: 1307 | GCCTCCCGTAGGAGT |
| | SEQ ID NO: 1308 |
| CTACGTAG | GCCTCCCTCGCGCCATCAGCTACGTAGCATGCT |
| SEQ ID NO: 1309 | GCCTCCCGTAGGAGT |
| | SEQ ID NO: 1310 |
| CTACGTTC | GCCTCCCTCGCGCCATCAGCTACGTTCCATGCT |
| SEQ ID NO: 1311 | GCCTCCCGTAGGAGT |
| | SEQ ID NO: 1312 |
| CTACTCCA | GCCTCCCTCGCGCCATCAGCTACTCCACATGCT |
| SEQ ID NO: 1313 | GCCTCCCGTAGGAGT |
| | SEQ ID NO: 1314 |
| CTACTCGT | GCCTCCCTCGCGCCATCAGCTACTCGTCATGCT |
| SEQ ID NO: 1315 | GCCTCCCGTAGGAGT |
| | SEQ ID NO: 1316 |
| CTACTGCT | GCCTCCCTCGCGCCATCAGCTACTGCTCATGCT |
| SEQ ID NO: 1317 | GCCTCCCGTAGGAGT |
| | SEQ ID NO: 1318 |
| CTACTGGA | GCCTCCCTCGCGCCATCAGCTACTGGACATGCT |
| SEQ ID NO: 1319 | GCCTCCCGTAGGAGT |
| | SEQ ID NO: 1320 |
| CTAGACCA | GCCTCCCTCGCGCCATCAGCTAGACCACATGCT |
| SEQ ID NO: 1321 | GCCTCCCGTAGGAGT |
| | SEQ ID NO: 1322 |
| CTAGACGT | GCCTCCCTCGCGCCATCAGCTAGACGTCATGCT |
| SEQ ID NO: 1323 | GCCTCCCGTAGGAGT |
| | SEQ ID NO: 1324 |
| CTAGAGCT | GCCTCCCTCGCGCCATCAGCTAGAGCTCATGCT |
| SEQ ID NO: 1325 | GCCTCCCGTAGGAGT |
| | SEQ ID NO: 1326 |
| CTAGAGGA | GCCTCCCTCGCGCCATCAGCTAGAGGACATGCT |
| SEQ ID NO: 1327 | GCCTCCCGTAGGAGT |
| | SEQ ID NO: 1328 |
| CTAGCAAC | GCCTCCCTCGCGCCATCAGCTAGCAACCATGCT |
| SEQ ID NO: 1329 | GCCTCCCGTAGGAGT |
| | SEQ ID NO: 1330 |
| CTAGCATG | GCCTCCCTCGCGCCATCAGCTAGCATGCATGCT |
| SEQ ID NO: 1331 | GCCTCCCGTAGGAGT |
| | SEQ ID NO: 1332 |
| CTAGCTAG | GCCTCCCTCGCGCCATCAGCTAGCTAGCATGCT |
| SEQ ID NO: 1333 | GCCTCCCGTAGGAGT |
| | SEQ ID NO: 1334 |
| CTAGCTTC | GCCTCCCTCGCGCCATCAGCTAGCTTCCATGCT |
| SEQ ID NO: 1335 | GCCTCCCGTAGGAGT |
| | SEQ ID NO: 1336 |
| CTAGGAAG | GCCTCCCTCGCGCCATCAGCTAGGAAGCATGCT |
| SEQ ID NO: 1337 | GCCTCCCGTAGGAGT |
| | SEQ ID NO: 1338 |
| CTAGGATC | GCCTCCCTCGCGCCATCAGCTAGGATCCATGCT |
| SEQ ID NO: 1339 | GCCTCCCGTAGGAGT |
| | SEQ ID NO: 1340 |
| CTAGGTAC | GCCTCCCTCGCGCCATCAGCTAGGTACCATGCT |
| SEQ ID NO: 1341 | GCCTCCCGTAGGAGT |
| | SEQ ID NO: 1342 |
| CTAGGTTG | GCCTCCCTCGCGCCATCAGCTAGGTTGCATGCT |
| SEQ ID NO: 1343 | GCCTCCCGTAGGAGT |
| | SEQ ID NO: 1344 |
| CTAGTCCT | GCCTCCCTCGCGCCATCAGCTAGTCCTCATGCT |
| SEQ ID NO: 1345 | GCCTCCCGTAGGAGT |
| | SEQ ID NO: 1346 |
| CTAGTCGA | GCCTCCCTCGCGCCATCAGCTAGTCGACATGCT |
| SEQ ID NO: 1347 | GCCTCCCGTAGGAGT |
| | SEQ ID NO: 1348 |
| CTAGTGCA | GCCTCCCTCGCGCCATCAGCTAGTGCACATGCT |
| SEQ ID NO: 1349 | GCCTCCCGTAGGAGT |
| | SEQ ID NO: 1350 |
| CTAGTGGT | GCCTCCCTCGCGCCATCAGCTAGTGGTCATGCT |
| SEQ ID NO: 1351 | GCCTCCCGTAGGAGT |
| | SEQ ID NO: 1352 |
| CTCAACAG | GCCTCCCTCGCGCCATCAGCTCAACAGCATGCT |
| SEQ ID NO: 1353 | GCCTCCCGTAGGAGT |
| | SEQ ID NO: 1354 |
| CTCAACTC | GCCTCCCTCGCGCCATCAGCTCAACTCCATGCT |
| SEQ ID NO: 1355 | GCCTCCCGTAGGAGT |
| | SEQ ID NO: 1356 |
| CTCAAGAC | GCCTCCCTCGCGCCATCAGCTCAAGACCATGCT |
| SEQ ID NO: 1357 | GCCTCCCGTAGGAGT |
| | SEQ ID NO: 1358 |
| CTCAAGTG | GCCTCCCTCGCGCCATCAGCTCAAGTGCATGCT |
| SEQ ID NO: 1359 | GCCTCCCGTAGGAGT |
| | SEQ ID NO: 1360 |
| CTCACACT | GCCTCCCTCGCGCCATCAGCTCACACTCATGCT |
| SEQ ID NO: 1361 | GCCTCCCGTAGGAGT |
| | SEQ ID NO: 1362 |
| CTCACAGA | GCCTCCCTCGCGCCATCAGCTCACAGACATGCT |
| SEQ ID NO: 1363 | GCCTCCCGTAGGAGT |
| | SEQ ID NO: 1364 |
| CTCACTCA | GCCTCCCTCGCGCCATCAGCTCACTCACATGCT |
| SEQ ID NO: 1365 | GCCTCCCGTAGGAGT |
| | SEQ ID NO: 1366 |
| CTCACTGT | GCCTCCCTCGCGCCATCAGCTCACTGTCATGCT |
| SEQ ID NO: 1367 | GCCTCCCGTAGGAGT |
| | SEQ ID NO: 1368 |
| CTCAGACA | GCCTCCCTCGCGCCATCAGCTCAGACACATGCT |
| SEQ ID NO: 1369 | GCCTCCCGTAGGAGT |
| | SEQ ID NO: 1370 |
| CTCAGAGT | GCCTCCCTCGCGCCATCAGCTCAGAGTCATGCT |
| SEQ ID NO: 1371 | GCCTCCCGTAGGAGT |
| | SEQ ID NO: 1372 |
| CTCAGTCT | GCCTCCCTCGCGCCATCAGCTCAGTCTCATGCT |
| SEQ ID NO: 1373 | GCCTCCCGTAGGAGT |
| | SEQ ID NO: 1374 |
| CTCAGTGA | GCCTCCCTCGCGCCATCAGCTCAGTGACATGCT |
| SEQ ID NO: 1375 | GCCTCCCGTAGGAGT |
| | SEQ ID NO: 1376 |
| CTCATCAC | GCCTCCCTCGCGCCATCAGCTCATCACCATGCT |
| SEQ ID NO: 1377 | GCCTCCCGTAGGAGT |
| | SEQ ID NO: 1378 |
| CTCATCTG | GCCTCCCTCGCGCCATCAGCTCATCTGCATGCT |
| SEQ ID NO: 1379 | GCCTCCCGTAGGAGT |
| | SEQ ID NO: 1380 |
| CTCATGAG | GCCTCCCTCGCGCCATCAGCTCATGAGCATGCT |
| SEQ ID NO: 1381 | GCCTCCCGTAGGAGT |
| | SEQ ID NO: 1382 |
| CTCATGTC | GCCTCCCTCGCGCCATCAGCTCATGTCCATGCT |
| SEQ ID NO: 1383 | GCCTCCCGTAGGAGT |
| | SEQ ID NO: 1384 |
| CTCTACAC | GCCTCCCTCGCGCCATCAGCTCTACACCATGCT |
| SEQ ID NO: 1385 | GCCTCCCGTAGGAGT |
| | SEQ ID NO: 1386 |
| CTCTACTG | GCCTCCCTCGCGCCATCAGCTCTACTGCATGCT |
| SEQ ID NO: 1387 | GCCTCCCGTAGGAGT |
| | SEQ ID NO: 1388 |
| CTCTAGAG | GCCTCCCTCGCGCCATCAGCTCTAGAGCATGCT |
| SEQ ID NO: 1389 | GCCTCCCGTAGGAGT |
| | SEQ ID NO: 1390 |
| CTCTAGTC | GCCTCCCTCGCGCCATCAGCTCTAGTCCATGCT |
| SEQ ID NO: 1391 | GCCTCCCGTAGGAGT |
| | SEQ ID NO: 1392 |
| CTCTCACA | GCCTCCCTCGCGCCATCAGCTCTCACACATGCT |
| SEQ ID NO: 1393 | GCCTCCCGTAGGAGT |
| | SEQ ID NO: 1394 |
| CTCTCAGT | GCCTCCCTCGCGCCATCAGCTCTCAGTCATGCT |
| SEQ ID NO: 1395 | GCCTCCCGTAGGAGT |
| | SEQ ID NO: 1396 |
| CTCTCTCT | GCCTCCCTCGCGCCATCAGCTCTCTCTCATGCT |
| SEQ ID NO: 1397 | GCCTCCCGTAGGAGT |
| | SEQ ID NO: 1398 |
| CTCTCTGA | GCCTCCCTCGCGCCATCAGCTCTCTGACATGCT |
| SEQ ID NO: 1399 | GCCTCCCGTAGGAGT |
| | SEQ ID NO: 1400 |
| CTCTGACT | GCCTCCCTCGCGCCATCAGCTCTGACTCATGCT |
| SEQ ID NO: 1401 | GCCTCCCGTAGGAGT |
| | SEQ ID NO: 1402 |
| CTCTGAGA | GCCTCCCTCGCGCCATCAGCTCTGAGACATGCT |
| SEQ ID NO: 1403 | GCCTCCCGTAGGAGT |
| | SEQ ID NO: 1404 |
| CTCTGTCA | GCCTCCCTCGCGCCATCAGCTCTGTCACATGCT |
| SEQ ID NO: 1405 | GCCTCCCGTAGGAGT |
| | SEQ ID NO: 1406 |
| CTCTGTGT | GCCTCCCTCGCGCCATCAGCTCTGTGTCATGCT |
| SEQ ID NO: 1407 | GCCTCCCGTAGGAGT |
| | SEQ ID NO: 1408 |
| CTCTTCAG | GCCTCCCTCGCGCCATCAGCTCTTCAGCATGCT |
| SEQ ID NO: 1409 | GCCTCCCGTAGGAGT |
| | SEQ ID NO: 1410 |
| CTCTTCTC | GCCTCCCTCGCGCCATCAGCTCTTCTCCATGCT |
| SEQ ID NO: 1411 | GCCTCCCGTAGGAGT |
| | SEQ ID NO: 1412 |
| CTCTTGAC | GCCTCCCTCGCGCCATCAGCTCTTGACCATGCT |
| SEQ ID NO: 1413 | GCCTCCCGTAGGAGT |
| | SEQ ID NO: 1414 |
| CTCTTGTG | GCCTCCCTCGCGCCATCAGCTCTTGTGCATGCT |
| SEQ ID NO: 1415 | GCCTCCCGTAGGAGT |
| | SEQ ID NO: 1416 |
| CTGAACAC | GCCTCCCTCGCGCCATCAGCTGAACACCATGCT |
| SEQ ID NO: 1417 | GCCTCCCGTAGGAGT |
| | SEQ ID NO: 1418 |
| CTGAACTG | GCCTCCCTCGCGCCATCAGCTGAACTGCATGCT |
| SEQ ID NO: 1419 | GCCTCCCGTAGGAGT |
| | SEQ ID NO: 1420 |
| CTGAAGAG | GCCTCCCTCGCGCCATCAGCTGAAGAGCATGCT |
| SEQ ID NO: 1421 | GCCTCCCGTAGGAGT |
| | SEQ ID NO: 1422 |
| CTGAAGTC | GCCTCCCTCGCGCCATCAGCTGAAGTCCATGCT |
| SEQ ID NO: 1423 | GCCTCCCGTAGGAGT |
| | SEQ ID NO: 1424 |
| CTGACACA | GCCTCCCTCGCGCCATCAGCTGACACACATGCT |
| SEQ ID NO: 1425 | GCCTCCCGTAGGAGT |
| | SEQ ID NO: 1426 |
| CTGACAGT | GCCTCCCTCGCGCCATCAGCTGACAGTCATGCT |
| SEQ ID NO: 1427 | GCCTCCCGTAGGAGT |
| | SEQ ID NO: 1428 |
| CTGACTCT | GCCTCCCTCGCGCCATCAGCTGACTCTCATGCT |
| SEQ ID NO: 1429 | GCCTCCCGTAGGAGT |
| | SEQ ID NO: 1430 |
| CTGACTGA | GCCTCCCTCGCGCCATCAGCTGACTGACATGCT |
| SEQ ID NO: 1431 | GCCTCCCGTAGGAGT |
| | SEQ ID NO: 1432 |
| CTGAGACT | GCCTCCCTCGCGCCATCAGCTGAGACTCATGCT |
| SEQ ID NO: 1433 | GCCTCCCGTAGGAGT |
| | SEQ ID NO: 1434 |
| CTGAGAGA | GCCTCCCTCGCGCCATCAGCTGAGAGACATGCT |
| SEQ ID NO: 1435 | GCCTCCCGTAGGAGT |
| | SEQ ID NO: 1436 |
| CTGAGTCA | GCCTCCCTCGCGCCATCAGCTGAGTCACATGCT |
| SEQ ID NO: 1437 | GCCTCCCGTAGGAGT |
| | SEQ ID NO: 1438 |
| CTGAGTGT | GCCTCCCTCGCGCCATCAGCTGAGTGTCATGCT |
| SEQ ID NO: 1439 | GCCTCCCGTAGGAGT |
| | SEQ ID NO: 1440 |
| CTGATCAG | GCCTCCCTCGCGCCATCAGCTGATCAGCATGCT |
| SEQ ID NO: 1441 | GCCTCCCGTAGGAGT |
| | SEQ ID NO: 1142 |
| CTGATCTC | GCCTCCCTCGCGCCATCAGCTGATCTCCATGCT |
| SEQ ID NO: 1143 | GCCTCCCGTAGGAGT |
| | SEQ ID NO: 1144 |
| CTGATGAC | GCCTCCCTCGCGCCATCAGCTGATGACCATGCT |
| SEQ ID NO: 1145 | GCCTCCCGTAGGAGT |
| | SEQ ID NO: 1146 |
| CTGATGTG | GCCTCCCTCGCGCCATCAGCTGATGTGCATGCT |
| SEQ ID NO: 1147 | GCCTCCCGTAGGAGT |
| | SEQ ID NO: 1148 |
| CTGTACAG | GCCTCCCTCGCGCCATCAGCTGTACAGCATGCT |
| SEQ ID NO: 1149 | GCCTCCCGTAGGAGT |
| | SEQ ID NO: 1150 |
| CTGTACTC | GCCTCCCTCGCGCCATCAGCTGTACTCCATGCT |
| SEQ ID NO: 1151 | GCCTCCCGTAGGAGT |
| | SEQ ID NO: 1152 |
| CTGTAGAC | GCCTCCCTCGCGCCATCAGCTGTAGACCATGCT |
| SEQ ID NO: 1153 | GCCTCCCGTAGGAGT |
| | SEQ ID NO: 1154 |
| CTGTAGTG | GCCTCCCTCGCGCCATCAGCTGTAGTGCATGCT |
| SEQ ID NO: 1155 | GCCTCCCGTAGGAGT |
| | SEQ ID NO: 1156 |
| CTGTCACT | GCCTCCCTCGCGCCATCAGCTGTCACTCATGCT |
| SEQ ID NO: 1157 | GCCTCCCGTAGGAGT |
| | SEQ ID NO: 1158 |
| CTGTCAGA | GCCTCCCTCGCGCCATCAGCTGTCAGACATGCT |
| SEQ ID NO: 1159 | GCCTCCCGTAGGAGT |
| | SEQ ID NO: 1160 |
| CTGTCTCA | GCCTCCCTCGCGCCATCAGCTGTCTCACATGCT |
| SEQ ID NO: 1161 | GCCTCCCGTAGGAGT |
| | SEQ ID NO: 1162 |
| CTGTCTGT | GCCTCCCTCGCGCCATCAGCTGTCTGTCATGCT |
| SEQ ID NO: 1163 | GCCTCCCGTAGGAGT |
| | SEQ ID NO: 1164 |
| CTGTGACA | GCCTCCCTCGCGCCATCAGCTGTGACACATGCT |
| SEQ ID NO: 1165 | GCCTCCCGTAGGAGT |
| | SEQ ID NO: 1166 |
| CTGTGAGT | GCCTCCCTCGCGCCATCAGCTGTGAGTCATGCT |
| SEQ ID NO: 1167 | GCCTCCCGTAGGAGT |
| | SEQ ID NO: 1168 |
| CTGTGTCT | GCCTCCCTCGCGCCATCAGCTGTGTCTCATGCT |
| SEQ ID NO: 1169 | GCCTCCCGTAGGAGT |
| | SEQ ID NO: 1170 |
| CTGTGTGA | GCCTCCCTCGCGCCATCAGCTGTGTGACATGCT |
| SEQ ID NO: 1171 | GCCTCCCGTAGGAGT |
| | SEQ ID NO: 1172 |
| CTGTTCAC | GCCTCCCTCGCGCCATCAGCTGTTCACCATGCT |
| SEQ ID NO: 1173 | GCCTCCCGTAGGAGT |
| | SEQ ID NO: 1174 |
| CTGTTCTG | GCCTCCCTCGCGCCATCAGCTGTTCTGCATGCT |
| SEQ ID NO: 1175 | GCCTCCCGTAGGAGT |
| | SEQ ID NO: 1176 |
| CTGTTGAG | GCCTCCCTCGCGCCATCAGCTGTTGAGCATGCT |
| SEQ ID NO: 1177 | GCCTCCCGTAGGAGT |
| | SEQ ID NO: 1178 |
| CTGTTGTC | GCCTCCCTCGCGCCATCAGCTGTTGTCCATGCT |
| SEQ ID NO: 1179 | GCCTCCCGTAGGAGT |
| | SEQ ID NO: 1180 |
| CTTCACCA | GCCTCCCTCGCGCCATCAGCTTCACCACATGCT |
| SEQ ID NO: 1181 | GCCTCCCGTAGGAGT |
| | SEQ ID NO: 1182 |
| CTTCACGT | GCCTCCCTCGCGCCATCAGCTTCACGTCATGCT |
| SEQ ID NO: 1183 | GCCTCCCGTAGGAGT |
| | SEQ ID NO: 1184 |
| CTTCAGCT | GCCTCCCTCGCGCCATCAGCTTCAGCTCATGCT |
| SEQ ID NO: 1185 | GCCTCCCGTAGGAGT |
| | SEQ ID NO: 1186 |
| CTTCAGGA | GCCTCCCTCGCGCCATCAGCTTCAGGACATGCT |
| SEQ ID NO: 1187 | GCCTCCCGTAGGAGT |
| | SEQ ID NO: 1188 |
| CTTCCAAC | GCCTCCCTCGCGCCATCAGCTTCCAACCATGCT |
| SEQ ID NO: 1189 | GCCTCCCGTAGGAGT |
| | SEQ ID NO: 1190 |
| CTTCCATG | GCCTCCCTCGCGCCATCAGCTTCCATGCATGCT |
| SEQ ID NO: 1191 | GCCTCCCGTAGGAGT |
| | SEQ ID NO: 1192 |
| CTTCCTAG | GCCTCCCTCGCGCCATCAGCTTCCTAGCATGCT |
| SEQ ID NO: 1193 | GCCTCCCGTAGGAGT |
| | SEQ ID NO: 1194 |
| CTTCCTTC | GCCTCCCTCGCGCCATCAGCTTCCTTCCATGCT |
| SEQ ID NO: 1195 | GCCTCCCGTAGGAGT |
| | SEQ ID NO: 1196 |
| CTTCGAAG | GCCTCCCTCGCGCCATCAGCTTCGAAGCATGCT |
| SEQ ID NO: 1197 | GCCTCCCGTAGGAGT |
| | SEQ ID NO: 1198 |
| CTTCGATC | GCCTCCCTCGCGCCATCAGCTTCGATCCATGCT |
| SEQ ID NO: 1199 | GCCTCCCGTAGGAGT |
| | SEQ ID NO: 1200 |
| CTTCGTAC | GCCTCCCTCGCGCCATCAGCTTCGTACCATGCT |
| SEQ ID NO: 1201 | GCCTCCCGTAGGAGT |
| | SEQ ID NO: 1202 |
| CTTCGTTG | GCCTCCCTCGCGCCATCAGCTTCGTTGCATGCT |
| SEQ ID NO: 1203 | GCCTCCCGTAGGAGT |
| | SEQ ID NO: 1204 |
| CTTCTCCT | GCCTCCCTCGCGCCATCAGCTTCTCCTCATGCT |
| SEQ ID NO: 1205 | GCCTCCCGTAGGAGT |
| | SEQ ID NO: 1206 |
| CTTCTCGA | GCCTCCCTCGCGCCATCAGCTTCTCGACATGCT |
| SEQ ID NO: 1207 | GCCTCCCGTAGGAGT |
| | SEQ ID NO: 1208 |
| CTTCTGCA | GCCTCCCTCGCGCCATCAGCTTCTGCACATGCT |
| SEQ ID NO: 1209 | GCCTCCCGTAGGAGT |
| | SEQ ID NO: 1210 |
| CTTCTGGT | GCCTCCCTCGCGCCATCAGCTTCTGGTCATGCT |
| SEQ ID NO: 1211 | GCCTCCCGTAGGAGT |
| | SEQ ID NO: 1212 |
| CTTGACCT | GCCTCCCTCGCGCCATCAGCTTGACCTCATGCT |
| SEQ ID NO: 1213 | GCCTCCCGTAGGAGT |
| | SEQ ID NO: 1214 |
| CTTGACGA | GCCTCCCTCGCGCCATCAGCTTGACGACATGCT |
| SEQ ID NO: 1215 | GCCTCCCGTAGGAGT |
| | SEQ ID NO: 1216 |
| CTTGAGCA | GCCTCCCTCGCGCCATCAGCTTGAGCACATGCT |
| SEQ ID NO: 1217 | GCCTCCCGTAGGAGT |
| | SEQ ID NO: 1218 |
| CTTGAGGT | GCCTCCCTCGCGCCATCAGCTTGAGGTCATGCT |
| SEQ ID NO: 1219 | GCCTCCCGTAGGAGT |
| | SEQ ID NO: 1220 |
| CTTGCAAG | GCCTCCCTCGCGCCATCAGCTTGCAAGCATGCT |
| SEQ ID NO: 1221 | GCCTCCCGTAGGAGT |
| | SEQ ID NO: 1222 |
| CTTGCATC | GCCTCCCTCGCGCCATCAGCTTGCATCCATGCT |
| SEQ ID NO: 1223 | GCCTCCCGTAGGAGT |
| | SEQ ID NO: 1224 |
| CTTGCTAC | GCCTCCCTCGCGCCATCAGCTTGCTACCATGCT |
| SEQ ID NO: 1225 | GCCTCCCGTAGGAGT |
| | SEQ ID NO: 1226 |
| CTTGCTTG | GCCTCCCTCGCGCCATCAGCTTGCTTGCATGCT |
| SEQ ID NO: 1227 | GCCTCCCGTAGGAGT |
| | SEQ ID NO: 1228 |
| CTTGGAAC | GCCTCCCTCGCGCCATCAGCTTGGAACCATGCT |
| SEQ ID NO: 1229 | GCCTCCCGTAGGAGT |
| | SEQ ID NO: 1230 |
| CTTGGATG | GCCTCCCTCGCGCCATCAGCTTGGATGCATGCT |
| SEQ ID NO: 1231 | GCCTCCCGTAGGAGT |
| | SEQ ID NO: 1232 |
| CTTGGTAG | GCCTCCCTCGCGCCATCAGCTTGGTAGCATGCT |
| SEQ ID NO: 1233 | GCCTCCCGTAGGAGT |
| | SEQ ID NO: 1234 |
| CTTGGTTC | GCCTCCCTCGCGCCATCAGCTTGGTTCCATGCT |
| SEQ ID NO: 1235 | GCCTCCCGTAGGAGT |
| | SEQ ID NO: 1236 |
| CTTGTCCA | GCCTCCCTCGCGCCATCAGCTTGTCCACATGCT |
| SEQ ID NO: 1237 | GCCTCCCGTAGGAGT |
| | SEQ ID NO: 1238 |
| CTTGTCGT | GCCTCCCTCGCGCCATCAGCTTGTCGTCATGCT |
| SEQ ID NO: 1239 | GCCTCCCGTAGGAGT |
| | SEQ ID NO: 1240 |
| CTTGTGCT | GCCTCCCTCGCGCCATCAGCTTGTGCTCATGCT |
| SEQ ID NO: 1241 | GCCTCCCGTAGGAGT |
| | SEQ ID NO: 1242 |
| CTTGTGGA | GCCTCCCTCGCGCCATCAGCTTGTGGACATGCT |
| SEQ ID NO: 1243 | GCCTCCCGTAGGAGT |
| | SEQ ID NO: 1244 |
| GAACACCT | GCCTCCCTCGCGCCATCAGGAACACCTCATGCT |
| SEQ ID NO: 1245 | GCCTCCCGTAGGAGT |
| | SEQ ID NO: 1246 |
| GAACACGA | GCCTCCCTCGCGCCATCAGGAACACGACATGCT |
| SEQ ID NO: 1247 | GCCTCCCGTAGGAGT |
| | SEQ ID NO: 1248 |
| GAACAGCA | GCCTCCCTCGCGCCATCAGGAACAGCACATGCT |
| SEQ ID NO: 1249 | GCCTCCCGTAGGAGT |
| | SEQ ID NO: 1250 |
| GAACAGGT | GCCTCCCTCGCGCCATCAGGAACAGGTCATGCT |
| SEQ ID NO: 1251 | GCCTCCCGTAGGAGT |
| | SEQ ID NO: 1252 |
| GAACCAAG | GCCTCCCTCGCGCCATCAGGAACCAAGCATGCT |
| SEQ ID NO: 1253 | GCCTCCCGTAGGAGT |
| | SEQ ID NO: 1254 |
| GAACCATC | GCCTCCCTCGCGCCATCAGGAACCATCCATGCT |
| SEQ ID NO: 1255 | GCCTCCCGTAGGAGT |
| | SEQ ID NO: 1256 |
| GAACCTAC | GCCTCCCTCGCGCCATCAGGAACCTACCATGCT |
| SEQ ID NO: 1257 | GCCTCCCGTAGGAGT |
| | SEQ ID NO: 1258 |
| GAACCTTG | GCCTCCCTCGCGCCATCAGGAACCTTGCATGCT |
| SEQ ID NO: 1259 | GCCTCCCGTAGGAGT |
| | SEQ ID NO: 1260 |
| GAACGAAC | GCCTCCCTCGCGCCATCAGGAACGAACCATGCT |
| SEQ ID NO: 1261 | GCCTCCCGTAGGAGT |
| | SEQ ID NO: 1262 |
| GAACGATG | GCCTCCCTCGCGCCATCAGGAACGATGCATGCT |
| SEQ ID NO: 1263 | GCCTCCCGTAGGAGT |
| | SEQ ID NO: 1264 |
| GAACGTAG | GCCTCCCTCGCGCCATCAGGAACGTAGCATGCT |
| SEQ ID NO: 1265 | GCCTCCCGTAGGAGT |
| | SEQ ID NO: 1266 |
| GAACGTTC | GCCTCCCTCGCGCCATCAGGAACGTTCCATGCT |
| SEQ ID NO: 1267 | GCCTCCCGTAGGAGT |
| | SEQ ID NO: 1268 |
| GAACTCCA | GCCTCCCTCGCGCCATCAGGAACTCCACATGCT |
| SEQ ID NO: 1269 | GCCTCCCGTAGGAGT |
| | SEQ ID NO: 1270 |
| GAACTCGT | GCCTCCCTCGCGCCATCAGGAACTCGTCATGCT |
| SEQ ID NO: 1271 | GCCTCCCGTAGGAGT |
| | SEQ ID NO: 1272 |
| GAACTGCT | GCCTCCCTCGCGCCATCAGGAACTGCTCATGCT |
| SEQ ID NO: 1273 | GCCTCCCGTAGGAGT |
| | SEQ ID NO: 1274 |
| GAACTGGA | GCCTCCCTCGCGCCATCAGGAACTGGACATGCT |
| SEQ ID NO: 1275 | GCCTCCCGTAGGAGT |
| | SEQ ID NO: 1276 |
| GAAGACCA | GCCTCCCTCGCGCCATCAGGAAGACCACATGCT |
| SEQ ID NO: 1277 | GCCTCCCGTAGGAGT |
| | SEQ ID NO: 1278 |
| GAAGACGT | GCCTCCCTCGCGCCATCAGGAAGACGTCATGCT |
| SEQ ID NO: 1279 | GCCTCCCGTAGGAGT |
| | SEQ ID NO: 1280 |
| GAAGAGCT | GCCTCCCTCGCGCCATCAGGAAGAGCTCATGCT |
| SEQ ID NO: 1281 | GCCTCCCGTAGGAGT |
| | SEQ ID NO: 1282 |
| GAAGAGGA | GCCTCCCTCGCGCCATCAGGAAGAGGACATGCT |
| SEQ ID NO: 1283 | GCCTCCCGTAGGAGT |
| | SEQ ID NO: 1284 |
| GAAGCAAC | GCCTCCCTCGCGCCATCAGGAAGCAACCATGCT |
| SEQ ID NO: 1285 | GCCTCCCGTAGGAGT |
| | SEQ ID NO: 1286 |
| GAAGCATG | GCCTCCCTCGCGCCATCAGGAAGCATGCATGCT |
| SEQ ID NO: 1287 | GCCTCCCGTAGGAGT |
| | SEQ ID NO: 1288 |
| GAAGCTAG | GCCTCCCTCGCGCCATCAGGAAGCTAGCATGCT |
| SEQ ID NO: 1289 | GCCTCCCGTAGGAGT |
| | SEQ ID NO: 1290 |
| GAAGCTTC | GCCTCCCTCGCGCCATCAGGAAGCTTCCATGCT |
| SEQ ID NO: 1291 | GCCTCCCGTAGGAGT |
| | SEQ ID NO: 1292 |
| GAAGGAAG | GCCTCCCTCGCGCCATCAGGAAGGAAGCATGCT |
| SEQ ID NO: 1293 | GCCTCCCGTAGGAGT |
| | SEQ ID NO: 1294 |
| GAAGGATC | GCCTCCCTCGCGCCATCAGGAAGGATCCATGCT |
| SEQ ID NO: 1295 | GCCTCCCGTAGGAGT |
| | SEQ ID NO: 1296 |
| GAAGGTAC | GCCTCCCTCGCGCCATCAGGAAGGTACCATGCT |
| SEQ ID NO: 1297 | GCCTCCCGTAGGAGT |
| | SEQ ID NO: 1298 |
| GAAGGTTG | GCCTCCCTCGCGCCATCAGGAAGGTTGCATGCT |
| SEQ ID NO: 1299 | GCCTCCCGTAGGAGT |
| | SEQ ID NO: 1300 |
| GAAGTCCT | GCCTCCCTCGCGCCATCAGGAAGTCCTCATGCT |
| SEQ ID NO: 1301 | GCCTCCCGTAGGAGT |
| | SEQ ID NO: 1302 |
| GAAGTCGA | GCCTCCCTCGCGCCATCAGGAAGTCGACATGCT |
| SEQ ID NO: 1303 | GCCTCCCGTAGGAGT |
| | SEQ ID NO: 1304 |
| GAAGTGCA | GCCTCCCTCGCGCCATCAGGAAGTGCACATGCT |
| SEQ ID NO: 1305 | GCCTCCCGTAGGAGT |
| | SEQ ID NO: 1306 |
| GAAGTGGT | GCCTCCCTCGCGCCATCAGGAAGTGGTCATGCT |
| SEQ ID NO: 1307 | GCCTCCCGTAGGAGT |
| | SEQ ID NO: 1308 |
| GACAACAG | GCCTCCCTCGCGCCATCAGGACAACAGCATGCT |
| SEQ ID NO: 1309 | GCCTCCCGTAGGAGT |
| | SEQ ID NO: 1310 |
| GACAACTC | GCCTCCCTCGCGCCATCAGGACAACTCCATGCT |
| SEQ ID NO: 1311 | GCCTCCCGTAGGAGT |
| | SEQ ID NO: 1312 |
| GACAAGAC | GCCTCCCTCGCGCCATCAGGACAAGACCATGCT |
| SEQ ID NO: 1313 | GCCTCCCGTAGGAGT |
| | SEQ ID NO: 1314 |
| GACAAGTG | GCCTCCCTCGCGCCATCAGGACAAGTGCATGCT |
| SEQ ID NO: 1315 | GCCTCCCGTAGGAGT |
| | SEQ ID NO: 1316 |
| GACACACT | GCCTCCCTCGCGCCATCAGGACACACTCATGCT |
| SEQ ID NO: 1317 | GCCTCCCGTAGGAGT |
| | SEQ ID NO: 1318 |
| GACACAGA | GCCTCCCTCGCGCCATCAGGACACAGACATGCT |
| SEQ ID NO: 1319 | GCCTCCCGTAGGAGT |
| | SEQ ID NO: 1320 |
| GACACTCA | GCCTCCCTCGCGCCATCAGGACACTCACATGCT |
| SEQ ID NO: 1321 | GCCTCCCGTAGGAGT |
| | SEQ ID NO: 1322 |
| GACACTGT | GCCTCCCTCGCGCCATCAGGACACTGTCATGCT |
| SEQ ID NO: 1323 | GCCTCCCGTAGGAGT |
| | SEQ ID NO: 1324 |
| GACAGACA | GCCTCCCTCGCGCCATCAGGACAGACACATGCT |
| SEQ ID NO: 1325 | GCCTCCCGTAGGAGT |
| | SEQ ID NO: 1326 |
| GACAGAGT | GCCTCCCTCGCGCCATCAGGACAGAGTCATGCT |
| SEQ ID NO: 1327 | GCCTCCCGTAGGAGT |
| | SEQ ID NO: 1328 |
| GACAGTCT | GCCTCCCTCGCGCCATCAGGACAGTCTCATGCT |
| SEQ ID NO: 1329 | GCCTCCCGTAGGAGT |
| | SEQ ID NO: 1330 |
| GACAGTGA | GCCTCCCTCGCGCCATCAGGACAGTGACATGCT |
| SEQ ID NO: 1331 | GCCTCCCGTAGGAGT |
| | SEQ ID NO: 1332 |
| GACATCAC | GCCTCCCTCGCGCCATCAGGACATCACCATGCT |
| SEQ ID NO: 1333 | GCCTCCCGTAGGAGT |
| | SEQ ID NO: 1334 |
| GACATCTG | GCCTCCCTCGCGCCATCAGGACATCTGCATGCT |
| SEQ ID NO: 1335 | GCCTCCCGTAGGAGT |
| | SEQ ID NO: 1336 |
| GACATGAG | GCCTCCCTCGCGCCATCAGGACATGAGCATGCT |
| SEQ ID NO: 1337 | GCCTCCCGTAGGAGT |
| | SEQ ID NO: 1338 |
| GACATGTC | GCCTCCCTCGCGCCATCAGGACATGTCCATGCT |
| SEQ ID NO: 1339 | GCCTCCCGTAGGAGT |
| | SEQ ID NO: 1340 |
| GACTACAC | GCCTCCCTCGCGCCATCAGGACTACACCATGCT |
| SEQ ID NO: 1341 | GCCTCCCGTAGGAGT |
| | SEQ ID NO: 1342 |
| GACTACTG | GCCTCCCTCGCGCCATCAGGACTACTGCATGCT |
| SEQ ID NO: 1343 | GCCTCCCGTAGGAGT |
| | SEQ ID NO: 1344 |
| GACTAGAG | GCCTCCCTCGCGCCATCAGGACTAGAGCATGCT |
| SEQ ID NO: 1345 | GCCTCCCGTAGGAGT |
| | SEQ ID NO: 1346 |
| GACTAGTC | GCCTCCCTCGCGCCATCAGGACTAGTCCATGCT |
| SEQ ID NO: 1347 | GCCTCCCGTAGGAGT |
| | SEQ ID NO: 1348 |
| GACTCACA | GCCTCCCTCGCGCCATCAGGACTCACACATGCT |
| SEQ ID NO: 1349 | GCCTCCCGTAGGAGT |
| | SEQ ID NO: 1350 |
| GACTCAGT | GCCTCCCTCGCGCCATCAGGACTCAGTCATGCT |
| SEQ ID NO: 1351 | GCCTCCCGTAGGAGT |
| | SEQ ID NO: 1352 |
| GACTCTCT | GCCTCCCTCGCGCCATCAGGACTCTCTCATGCT |
| SEQ ID NO: 1353 | GCCTCCCGTAGGAGT |
| | SEQ ID NO: 1354 |
| GACTCTGA | GCCTCCCTCGCGCCATCAGGACTCTGACATGCT |
| SEQ ID NO: 1355 | GCCTCCCGTAGGAGT |
| | SEQ ID NO: 1356 |
| GACTGACT | GCCTCCCTCGCGCCATCAGGACTGACTCATGCT |
| SEQ ID NO: 1357 | GCCTCCCGTAGGAGT |
| | SEQ ID NO: 1358 |
| GACTGAGA | GCCTCCCTCGCGCCATCAGGACTGAGACATGCT |
| SEQ ID NO: 1359 | GCCTCCCGTAGGAGT |
| | SEQ ID NO: 1360 |
| GACTGTCA | GCCTCCCTCGCGCCATCAGGACTGTCACATGCT |
| SEQ ID NO: 1361 | GCCTCCCGTAGGAGT |
| | SEQ ID NO: 1362 |
| GACTGTGT | GCCTCCCTCGCGCCATCAGGACTGTGTCATGCT |
| SEQ ID NO: 1363 | GCCTCCCGTAGGAGT |
| | SEQ ID NO: 1364 |
| GACTTCAG | GCCTCCCTCGCGCCATCAGGACTTCAGCATGCT |
| SEQ ID NO: 1365 | GCCTCCCGTAGGAGT |
| | SEQ ID NO: 1366 |
| GACTTCTC | GCCTCCCTCGCGCCATCAGGACTTCTCCATGCT |
| SEQ ID NO: 1367 | GCCTCCCGTAGGAGT |
| | SEQ ID NO: 1368 |
| GACTTGAC | GCCTCCCTCGCGCCATCAGGACTTGACCATGCT |
| SEQ ID NO: 1369 | GCCTCCCGTAGGAGT |
| | SEQ ID NO: 1370 |
| GACTTGTG | GCCTCCCTCGCGCCATCAGGACTTGTGCATGCT |
| SEQ ID NO: 1371 | GCCTCCCGTAGGAGT |
| | SEQ ID NO: 1372 |
| GAGAACAC | GCCTCCCTCGCGCCATCAGGAGAACACCATGCT |
| SEQ ID NO: 1373 | GCCTCCCGTAGGAGT |
| | SEQ ID NO: 1374 |
| GAGAACTG | GCCTCCCTCGCGCCATCAGGAGAACTGCATGCT |
| SEQ ID NO: 1375 | GCCTCCCGTAGGAGT |
| | SEQ ID NO: 1376 |
| GAGAAGAG | GCCTCCCTCGCGCCATCAGGAGAAGAGCATGCT |
| SEQ ID NO: 1377 | GCCTCCCGTAGGAGT |
| | SEQ ID NO: 1378 |
| GAGAAGTC | GCCTCCCTCGCGCCATCAGGAGAAGTCCATGCT |
| SEQ ID NO: 1379 | GCCTCCCGTAGGAGT |
| | SEQ ID NO: 1380 |
| GAGACACA | GCCTCCCTCGCGCCATCAGGAGACACACATGCT |
| SEQ ID NO: 1381 | GCCTCCCGTAGGAGT |
| | SEQ ID NO: 1382 |
| GAGACAGT | GCCTCCCTCGCGCCATCAGGAGACAGTCATGCT |
| SEQ ID NO: 1383 | GCCTCCCGTAGGAGT |
| | SEQ ID NO: 1384 |
| GAGACTCT | GCCTCCCTCGCGCCATCAGGAGACTCTCATGCT |
| SEQ ID NO: 1385 | GCCTCCCGTAGGAGT |
| | SEQ ID NO: 1386 |
| GAGACTGA | GCCTCCCTCGCGCCATCAGGAGACTGACATGCT |
| SEQ ID NO: 1387 | GCCTCCCGTAGGAGT |
| | SEQ ID NO: 1388 |
| GAGAGACT | GCCTCCCTCGCGCCATCAGGAGAGACTCATGCT |
| SEQ ID NO: 1389 | GCCTCCCGTAGGAGT |
| | SEQ ID NO: 1390 |
| GAGAGAGA | GCCTCCCTCGCGCCATCAGGAGAGAGACATGCT |
| SEQ ID NO: 1391 | GCCTCCCGTAGGAGT |
| | SEQ ID NO: 1392 |
| GAGAGTCA | GCCTCCCTCGCGCCATCAGGAGAGTCACATGCT |
| SEQ ID NO: 1393 | GCCTCCCGTAGGAGT |
| | SEQ ID NO: 1394 |
| GAGAGTGT | GCCTCCCTCGCGCCATCAGGAGAGTGTCATGCT |
| SEQ ID NO: 1395 | GCCTCCCGTAGGAGT |
| | SEQ ID NO: 1396 |
| GAGATCAG | GCCTCCCTCGCGCCATCAGGAGATCAGCATGCT |
| SEQ ID NO: 1397 | GCCTCCCGTAGGAGT |
| | SEQ ID NO: 1398 |
| GAGATCTC | GCCTCCCTCGCGCCATCAGGAGATCTCCATGCT |
| SEQ ID NO: 1399 | GCCTCCCGTAGGAGT |
| | SEQ ID NO: 1400 |
| GAGATGAC | GCCTCCCTCGCGCCATCAGGAGATGACCATGCT |
| SEQ ID NO: 1401 | GCCTCCCGTAGGAGT |
| | SEQ ID NO: 1402 |
| GAGATGTG | GCCTCCCTCGCGCCATCAGGAGATGTGCATGCT |
| SEQ ID NO: 1403 | GCCTCCCGTAGGAGT |
| | SEQ ID NO: 1404 |
| GAGTACAG | GCCTCCCTCGCGCCATCAGGAGTACAGCATGCT |
| SEQ ID NO: 1405 | GCCTCCCGTAGGAGT |
| | SEQ ID NO: 1406 |
| GAGTACTC | GCCTCCCTCGCGCCATCAGGAGTACTCCATGCT |
| SEQ ID NO: 1407 | GCCTCCCGTAGGAGT |
| | SEQ ID NO: 1408 |
| GAGTAGAC | GCCTCCCTCGCGCCATCAGGAGTAGACCATGCT |
| SEQ ID NO: 1409 | GCCTCCCGTAGGAGT |
| | SEQ ID NO: 1410 |
| GAGTAGTG | GCCTCCCTCGCGCCATCAGGAGTAGTGCATGCT |
| SEQ ID NO: 1411 | GCCTCCCGTAGGAGT |
| | SEQ ID NO: 1412 |
| GAGTCACT | GCCTCCCTCGCGCCATCAGGAGTCACTCATGCT |
| SEQ ID NO: 1413 | GCCTCCCGTAGGAGT |
| | SEQ ID NO: 1414 |
| GAGTCAGA | GCCTCCCTCGCGCCATCAGGAGTCAGACATGCT |
| SEQ ID NO: 1415 | GCCTCCCGTAGGAGT |
| | SEQ ID NO: 1416 |
| GAGTCTCA | GCCTCCCTCGCGCCATCAGGAGTCTCACATGCT |
| SEQ ID NO: 1417 | GCCTCCCGTAGGAGT |
| | SEQ ID NO: 1418 |
| GAGTCTGT | GCCTCCCTCGCGCCATCAGGAGTCTGTCATGCT |
| SEQ ID NO: 1419 | GCCTCCCGTAGGAGT |
| | SEQ ID NO: 1420 |
| GAGTGACA | GCCTCCCTCGCGCCATCAGGAGTGACACATGCT |
| SEQ ID NO: 1421 | GCCTCCCGTAGGAGT |
| | SEQ ID NO: 1422 |
| GAGTGAGT | GCCTCCCTCGCGCCATCAGGAGTGAGTCATGCT |
| SEQ ID NO: 1423 | GCCTCCCGTAGGAGT |
| | SEQ ID NO: 1424 |
| GAGTGTCT | GCCTCCCTCGCGCCATCAGGAGTGTCTCATGCT |
| SEQ ID NO: 1425 | GCCTCCCGTAGGAGT |
| | SEQ ID NO: 1426 |
| GAGTGTGA | GCCTCCCTCGCGCCATCAGGAGTGTGACATGCT |
| SEQ ID NO: 1427 | GCCTCCCGTAGGAGT |
| | SEQ ID NO: 1428 |
| GAGTTCAC | GCCTCCCTCGCGCCATCAGGAGTTCACCATGCT |
| SEQ ID NO: 1429 | GCCTCCCGTAGGAGT |
| | SEQ ID NO: 1430 |
| GAGTTCTG | GCCTCCCTCGCGCCATCAGGAGTTCTGCATGCT |
| SEQ ID NO: 1431 | GCCTCCCGTAGGAGT |
| | SEQ ID NO: 1432 |
| GAGTTGAG | GCCTCCCTCGCGCCATCAGGAGTTGAGCATGCT |
| SEQ ID NO: 1433 | GCCTCCCGTAGGAGT |
| | SEQ ID NO: 1434 |
| GAGTTGTC | GCCTCCCTCGCGCCATCAGGAGTTGTCCATGCT |
| SEQ ID NO: 1435 | GCCTCCCGTAGGAGT |
| | SEQ ID NO: 1436 |
| GATCACCA | GCCTCCCTCGCGCCATCAGGATCACCACATGCT |
| SEQ ID NO: 1437 | GCCTCCCGTAGGAGT |
| | SEQ ID NO: 1438 |
| GATCACGT | GCCTCCCTCGCGCCATCAGGATCACGTCATGCT |
| SEQ ID NO: 1439 | GCCTCCCGTAGGAGT |
| | SEQ ID NO: 1440 |
| GATCAGCT | GCCTCCCTCGCGCCATCAGGATCAGCTCATGCT |
| SEQ ID NO: 1441 | GCCTCCCGTAGGAGT |
| | SEQ ID NO: 1442 |
| GATCAGGA | GCCTCCCTCGCGCCATCAGGATCAGGACATGCT |
| SEQ ID NO: 1443 | GCCTCCCGTAGGAGT |
| | SEQ ID NO: 1444 |
| GATCCAAC | GCCTCCCTCGCGCCATCAGGATCCAACCATGCT |
| SEQ ID NO: 1445 | GCCTCCCGTAGGAGT |
| | SEQ ID NO: 1446 |
| GATCCATG | GCCTCCCTCGCGCCATCAGGATCCATGCATGCT |
| SEQ ID NO: 1447 | GCCTCCCGTAGGAGT |
| | SEQ ID NO: 1448 |
| GATCCTAG | GCCTCCCTCGCGCCATCAGGATCCTAGCATGCT |
| SEQ ID NO: 1449 | GCCTCCCGTAGGAGT |
| | SEQ ID NO: 1450 |
| GATCCTTC | GCCTCCCTCGCGCCATCAGGATCCTTCCATGCT |
| SEQ ID NO: 1451 | GCCTCCCGTAGGAGT |
| | SEQ ID NO: 1452 |
| GATCGAAG | GCCTCCCTCGCGCCATCAGGATCGAAGCATGCT |
| SEQ ID NO: 1453 | GCCTCCCGTAGGAGT |
| | SEQ ID NO: 1454 |
| GATCGATC | GCCTCCCTCGCGCCATCAGGATCGATCCATGCT |
| SEQ ID NO: 1455 | GCCTCCCGTAGGAGT |
| | SEQ ID NO: 1456 |
| GATCGTAC | GCCTCCCTCGCGCCATCAGGATCGTACCATGCT |
| SEQ ID NO: 1457 | GCCTCCCGTAGGAGT |
| | SEQ ID NO: 1458 |
| GATCGTTG | GCCTCCCTCGCGCCATCAGGATCGTTGCATGCT |
| SEQ ID NO: 1459 | GCCTCCCGTAGGAGT |
| | SEQ ID NO: 1460 |
| GATCTCCT | GCCTCCCTCGCGCCATCAGGATCTCCTCATGCT |
| SEQ ID NO: 1461 | GCCTCCCGTAGGAGT |
| | SEQ ID NO: 1462 |
| GATCTCGA | GCCTCCCTCGCGCCATCAGGATCTCGACATGCT |
| SEQ ID NO: 1463 | GCCTCCCGTAGGAGT |
| | SEQ ID NO: 1464 |
| GATCTGCA | GCCTCCCTCGCGCCATCAGGATCTGCACATGCT |
| SEQ ID NO: 1465 | GCCTCCCGTAGGAGT |
| | SEQ ID NO: 1466 |
| GATCTGGT | GCCTCCCTCGCGCCATCAGGATCTGGTCATGCT |
| SEQ ID NO: 1467 | GCCTCCCGTAGGAGT |
| | SEQ ID NO: 1468 |
| GATGACCT | GCCTCCCTCGCGCCATCAGGATGACCTCATGCT |
| SEQ ID NO: 1469 | GCCTCCCGTAGGAGT |
| | SEQ ID NO: 1470 |
| GATGACGA | GCCTCCCTCGCGCCATCAGGATGACGACATGCT |
| SEQ ID NO: 1471 | GCCTCCCGTAGGAGT |
| | SEQ ID NO: 1472 |
| GATGAGCA | GCCTCCCTCGCGCCATCAGGATGAGCACATGCT |
| SEQ ID NO: 1473 | GCCTCCCGTAGGAGT |
| | SEQ ID NO: 1474 |
| GATGAGGT | GCCTCCCTCGCGCCATCAGGATGAGGTCATGCT |
| SEQ ID NO: 1475 | GCCTCCCGTAGGAGT |
| | SEQ ID NO: 1476 |
| GATGCAAG | GCCTCCCTCGCGCCATCAGGATGCAAGCATGCT |
| SEQ ID NO: 1477 | GCCTCCCGTAGGAGT |
| | SEQ ID NO: 1478 |
| GATGCATC | GCCTCCCTCGCGCCATCAGGATGCATCCATGCT |
| SEQ ID NO: 1479 | GCCTCCCGTAGGAGT |
| | SEQ ID NO: 1480 |
| GATGCTAC | GCCTCCCTCGCGCCATCAGGATGCTACCATGCT |
| SEQ ID NO: 1481 | GCCTCCCGTAGGAGT |
| | SEQ ID NO: 1482 |
| GATGCTTG | GCCTCCCTCGCGCCATCAGGATGCTTGCATGCT |
| SEQ ID NO: 1483 | GCCTCCCGTAGGAGT |
| | SEQ ID NO: 1484 |
| GATGGAAC | GCCTCCCTCGCGCCATCAGGATGGAACCATGCT |
| SEQ ID NO: 1485 | GCCTCCCGTAGGAGT |
| | SEQ ID NO: 1486 |
| GATGGATG | GCCTCCCTCGCGCCATCAGGATGGATGCATGCT |
| SEQ ID NO: 1487 | GCCTCCCGTAGGAGT |
| | SEQ ID NO: 1488 |
| GATGGTAG | GCCTCCCTCGCGCCATCAGGATGGTAGCATGCT |
| SEQ ID NO: 1489 | GCCTCCCGTAGGAGT |
| | SEQ ID NO: 1490 |
| GATGGTTC | GCCTCCCTCGCGCCATCAGGATGGTTCCATGCT |
| SEQ ID NO: 1491 | GCCTCCCGTAGGAGT |
| | SEQ ID NO: 1492 |
| GATGTCCA | GCCTCCCTCGCGCCATCAGGATGTCCACATGCT |
| SEQ ID NO: 1493 | GCCTCCCGTAGGAGT |
| | SEQ ID NO: 1494 |
| GATGTCGT | GCCTCCCTCGCGCCATCAGGATGTCGTCATGCT |
| SEQ ID NO: 1495 | GCCTCCCGTAGGAGT |
| | SEQ ID NO: 1496 |
| GATGTGCT | GCCTCCCTCGCGCCATCAGGATGTGCTCATGCT |
| SEQ ID NO: 1497 | GCCTCCCGTAGGAGT |
| | SEQ ID NO: 1498 |
| GATGTGGA | GCCTCCCTCGCGCCATCAGGATGTGGACATGCT |
| SEQ ID NO: 1499 | GCCTCCCGTAGGAGT |
| | SEQ ID NO: 1500 |
| GCAACCAT | GCCTCCCTCGCGCCATCAGGCAACCATCATGCT |
| SEQ ID NO: 1501 | GCCTCCCGTAGGAGT |
| | SEQ ID NO: 1502 |
| GCAACCTA | GCCTCCCTCGCGCCATCAGGCAACCTACATGCT |
| SEQ ID NO: 1503 | GCCTCCCGTAGGAGT |
| | SEQ ID NO: 1504 |
| GCAACGAA | GCCTCCCTCGCGCCATCAGGCAACGAACATGCT |
| SEQ ID NO: 1505 | GCCTCCCGTAGGAGT |
| | SEQ ID NO: 1506 |
| GCAACGTT | GCCTCCCTCGCGCCATCAGGCAACGTTCATGCT |
| SEQ ID NO: 1507 | GCCTCCCGTAGGAGT |
| | SEQ ID NO: 1508 |
| GCAAGCAA | GCCTCCCTCGCGCCATCAGGCAAGCAACATGCT |
| SEQ ID NO: 1509 | GCCTCCCGTAGGAGT |
| | SEQ ID NO: 1510 |
| GCAAGCTT | GCCTCCCTCGCGCCATCAGGCAAGCTTCATGCT |
| SEQ ID NO: 1511 | GCCTCCCGTAGGAGT |
| | SEQ ID NO: 1512 |
| GCAAGGAT | GCCTCCCTCGCGCCATCAGGCAAGGATCATGCT |
| SEQ ID NO: 1513 | GCCTCCCGTAGGAGT |
| | SEQ ID NO: 1514 |
| GCAAGGTA | GCCTCCCTCGCGCCATCAGGCAAGGTACATGCT |
| SEQ ID NO: 1515 | GCCTCCCGTAGGAGT |
| | SEQ ID NO: 1516 |
| GCAATACC | GCCTCCCTCGCGCCATCAGGCAATACCCATGCT |
| SEQ ID NO: 1517 | GCCTCCCGTAGGAGT |
| | SEQ ID NO: 1518 |
| GCAATAGG | GCCTCCCTCGCGCCATCAGGCAATAGGCATGCT |
| SEQ ID NO: 1519 | GCCTCCCGTAGGAGT |
| | SEQ ID NO: 1520 |
| GCAATTCG | GCCTCCCTCGCGCCATCAGGCAATTCGCATGCT |
| SEQ ID NO: 1521 | GCCTCCCGTAGGAGT |
| | SEQ ID NO: 1522 |
| GCAATTGC | GCCTCCCTCGCGCCATCAGGCAATTGCCATGCT |
| SEQ ID NO: 1523 | GCCTCCCGTAGGAGT |
| | SEQ ID NO: 1524 |
| GCATAACC | GCCTCCCTCGCGCCATCAGGCATAACCCATGCT |
| SEQ ID NO: 1525 | GCCTCCCGTAGGAGT |
| | SEQ ID NO: 1526 |
| GCATAAGG | GCCTCCCTCGCGCCATCAGGCATAAGGCATGCT |
| SEQ ID NO: 1527 | GCCTCCCGTAGGAGT |
| | SEQ ID NO: 1528 |
| GCATATCG | GCCTCCCTCGCGCCATCAGGCATATCGCATGCT |
| SEQ ID NO: 1529 | GCCTCCCGTAGGAGT |
| | SEQ ID NO: 1530 |
| GCATATGC | GCCTCCCTCGCGCCATCAGGCATATGCCATGCT |
| SEQ ID NO: 1531 | GCCTCCCGTAGGAGT |
| | SEQ ID NO: 1532 |
| GCATCCAA | GCCTCCCTCGCGCCATCAGGCATCCAACATGCT |
| SEQ ID NO: 1533 | GCCTCCCGTAGGAGT |
| | SEQ ID NO: 1534 |
| GCATCCTT | GCCTCCCTCGCGCCATCAGGCATCCTTCATGCT |
| SEQ ID NO: 1535 | GCCTCCCGTAGGAGT |
| | SEQ ID NO: 1536 |
| GCATCGAT | GCCTCCCTCGCGCCATCAGGCATCGATCATGCT |
| SEQ ID NO: 1537 | GCCTCCCGTAGGAGT |
| | SEQ ID NO: 1538 |
| GCATCGTA | GCCTCCCTCGCGCCATCAGGCATCGTACATGCT |
| SEQ ID NO: 1539 | GCCTCCCGTAGGAGT |
| | SEQ ID NO: 1540 |
| GCATGCAT | GCCTCCCTCGCGCCATCAGGCATGCATCATGCT |
| SEQ ID NO: 1541 | GCCTCCCGTAGGAGT |
| | SEQ ID NO: 1542 |
| GCATGCTA | GCCTCCCTCGCGCCATCAGGCATGCTACATGCT |
| SEQ ID NO: 1543 | GCCTCCCGTAGGAGT |
| | SEQ ID NO: 1544 |
| GCATGGAA | GCCTCCCTCGCGCCATCAGGCATGGAACATGCT |
| SEQ ID NO: 1545 | GCCTCCCGTAGGAGT |
| | SEQ ID NO: 1546 |
| GCATGGTT | GCCTCCCTCGCGCCATCAGGCATGGTTCATGCT |
| SEQ ID NO: 1547 | GCCTCCCGTAGGAGT |
| | SEQ ID NO: 1548 |
| GCATTACG | GCCTCCCTCGCGCCATCAGGCATTACGCATGCT |
| SEQ ID NO: 1549 | GCCTCCCGTAGGAGT |
| | SEQ ID NO: 1550 |
| GCATTAGC | GCCTCCCTCGCGCCATCAGGCATTAGCCATGCT |
| SEQ ID NO: 1551 | GCCTCCCGTAGGAGT |
| | SEQ ID NO: 1552 |
| GCCGAATT | GCCTCCCTCGCGCCATCAGGCCGAATTCATGCT |
| SEQ ID NO: 1553 | GCCTCCCGTAGGAGT |
| | SEQ ID NO: 1554 |
| GCCGATAT | GCCTCCCTCGCGCCATCAGGCCGATATCATGCT |
| SEQ ID NO: 1555 | GCCTCCCGTAGGAGT |
| | SEQ ID NO: 1556 |
| GCCGATTA | GCCTCCCTCGCGCCATCAGGCCGATTACATGCT |
| SEQ ID NO: 1557 | GCCTCCCGTAGGAGT |
| | SEQ ID NO: 1558 |
| GCCGTAAT | GCCTCCCTCGCGCCATCAGGCCGTAATCATGCT |
| SEQ ID NO: 1559 | GCCTCCCGTAGGAGT |
| | SEQ ID NO: 1560 |
| GCCGTATA | GCCTCCCTCGCGCCATCAGGCCGTATACATGCT |
| SEQ ID NO: 1561 | GCCTCCCGTAGGAGT |
| | SEQ ID NO: 1562 |
| GCCGTTAA | GCCTCCCTCGCGCCATCAGGCCGTTAACATGCT |
| SEQ ID NO: 1563 | GCCTCCCGTAGGAGT |
| | SEQ ID NO: 1564 |
| GCGCAATT | GCCTCCCTCGCGCCATCAGGCGCAATTCATGCT |
| SEQ ID NO: 1565 | GCCTCCCGTAGGAGT |
| | SEQ ID NO: 1566 |
| GCGCATAT | GCCTCCCTCGCGCCATCAGGCGCATATCATGCT |
| SEQ ID NO: 1567 | GCCTCCCGTAGGAGT |
| | SEQ ID NO: 1568 |
| GCGCATTA | GCCTCCCTCGCGCCATCAGGCGCATTACATGCT |
| SEQ ID NO: 1569 | GCCTCCCGTAGGAGT |
| | SEQ ID NO: 1570 |
| GCGCTAAT | GCCTCCCTCGCGCCATCAGGCGCTAATCATGCT |
| SEQ ID NO: 1571 | GCCTCCCGTAGGAGT |
| | SEQ ID NO: 1572 |
| GCGCTATA | GCCTCCCTCGCGCCATCAGGCGCTATACATGCT |
| SEQ ID NO: 1573 | GCCTCCCGTAGGAGT |
| | SEQ ID NO: 1574 |
| GCGCTTAA | GCCTCCCTCGCGCCATCAGGCGCTTAACATGCT |
| SEQ ID NO: 1575 | GCCTCCCGTAGGAGT |
| | SEQ ID NO: 1576 |
| GCGGAATA | GCCTCCCTCGCGCCATCAGGCGGAATACATGCT |
| SEQ ID NO: 1577 | GCCTCCCGTAGGAGT |
| | SEQ ID NO: 1578 |
| GCGGATAA | GCCTCCCTCGCGCCATCAGGCGGATAACATGCT |
| SEQ ID NO: 1579 | GCCTCCCGTAGGAGT |
| | SEQ ID NO: 1580 |
| GCGGTATT | GCCTCCCTCGCGCCATCAGGCGGTATTCATGCT |
| SEQ ID NO: 1581 | GCCTCCCGTAGGAGT |
| | SEQ ID NO: 1582 |
| GCGGTTAT | GCCTCCCTCGCGCCATCAGGCGGTTATCATGCT |
| SEQ ID NO: 1583 | GCCTCCCGTAGGAGT |
| | SEQ ID NO: 1584 |
| GCTAATCG | GCCTCCCTCGCGCCATCAGGCTAATCGCATGCT |
| SEQ ID NO: 1585 | GCCTCCCGTAGGAGT |
| | SEQ ID NO: 1586 |
| GCTAATGC | GCCTCCCTCGCGCCATCAGGCTAATGCCATGCT |
| SEQ ID NO: 1587 | GCCTCCCGTAGGAGT |
| | SEQ ID NO: 1588 |
| GCTACCAA | GCCTCCCTCGCGCCATCAGGCTACCAACATGCT |
| SEQ ID NO: 1589 | GCCTCCCGTAGGAGT |
| | SEQ ID NO: 1590 |
| GCTACCTT | GCCTCCCTCGCGCCATCAGGCTACCTTCATGCT |
| SEQ ID NO: 1591 | GCCTCCCGTAGGAGT |
| | SEQ ID NO: 1592 |
| GCTACGAT | GCCTCCCTCGCGCCATCAGGCTACGATCATGCT |
| SEQ ID NO: 1593 | GCCTCCCGTAGGAGT |
| | SEQ ID NO: 1594 |
| GCTACGTA | GCCTCCCTCGCGCCATCAGGCTACGTACATGCT |
| SEQ ID NO: 1595 | GCCTCCCGTAGGAGT |
| | SEQ ID NO: 1596 |
| GCTAGCAT | GCCTCCCTCGCGCCATCAGGCTAGCATCATGCT |
| SEQ ID NO: 1597 | GCCTCCCGTAGGAGT |
| | SEQ ID NO: 1598 |
| GCTAGCTA | GCCTCCCTCGCGCCATCAGGCTAGCTACATGCT |
| SEQ ID NO: 1599 | GCCTCCCGTAGGAGT |
| | SEQ ID NO: 1600 |
| GCTAGGAA | GCCTCCCTCGCGCCATCAGGCTAGGAACATGCT |
| SEQ ID NO: 1601 | GCCTCCCGTAGGAGT |
| | SEQ ID NO: 1602 |
| GCTAGGTT | GCCTCCCTCGCGCCATCAGGCTAGGTTCATGCT |
| SEQ ID NO: 1603 | GCCTCCCGTAGGAGT |
| | SEQ ID NO: 1604 |
| GCTATACG | GCCTCCCTCGCGCCATCAGGCTATACGCATGCT |
| SEQ ID NO: 1605 | GCCTCCCGTAGGAGT |
| | SEQ ID NO: 1606 |
| GCTATAGC | GCCTCCCTCGCGCCATCAGGCTATAGCCATGCT |
| SEQ ID NO: 1607 | GCCTCCCGTAGGAGT |
| | SEQ ID NO: 1608 |
| GCTATTCC | GCCTCCCTCGCGCCATCAGGCTATTCCCATGCT |
| SEQ ID NO: 1609 | GCCTCCCGTAGGAGT |
| | SEQ ID NO: 1610 |
| GCTATTGG | GCCTCCCTCGCGCCATCAGGCTATTGGCATGCT |
| SEQ ID NO: 1611 | GCCTCCCGTAGGAGT |
| | SEQ ID NO: 1612 |
| GCTTAACG | GCCTCCCTCGCGCCATCAGGCTTAACGCATGCT |
| SEQ ID NO: 1613 | GCCTCCCGTAGGAGT |
| | SEQ ID NO: 1614 |
| GCTTAAGC | GCCTCCCTCGCGCCATCAGGCTTAAGCCATGCT |
| SEQ ID NO: 1615 | GCCTCCCGTAGGAGT |
| | SEQ ID NO: 1616 |
| GCTTATCC | GCCTCCCTCGCGCCATCAGGCTTATCCCATGCT |
| SEQ ID NO: 1617 | GCCTCCCGTAGGAGT |
| | SEQ ID NO: 1618 |
| GCTTATGG | GCCTCCCTCGCGCCATCAGGCTTATGGCATGCT |
| SEQ ID NO: 1619 | GCCTCCCGTAGGAGT |
| | SEQ ID NO: 1620 |
| GCTTCCAT | GCCTCCCTCGCGCCATCAGGCTTCCATCATGCT |
| SEQ ID NO: 1621 | GCCTCCCGTAGGAGT |
| | SEQ ID NO: 1622 |
| GCTTCCTA | GCCTCCCTCGCGCCATCAGGCTTCCTACATGCT |
| SEQ ID NO: 1623 | GCCTCCCGTAGGAGT |
| | SEQ ID NO: 1624 |
| GCTTCGAA | GCCTCCCTCGCGCCATCAGGCTTCGAACATGCT |
| SEQ ID NO: 1625 | GCCTCCCGTAGGAGT |
| | SEQ ID NO: 1626 |
| GCTTCGTT | GCCTCCCTCGCGCCATCAGGCTTCGTTCATGCT |
| SEQ ID NO: 1627 | GCCTCCCGTAGGAGT |
| | SEQ ID NO: 1628 |
| GCTTGCAA | GCCTCCCTCGCGCCATCAGGCTTGCAACATGCT |
| SEQ ID NO: 1629 | GCCTCCCGTAGGAGT |
| | SEQ ID NO: 1630 |
| GCTTGCTT | GCCTCCCTCGCGCCATCAGGCTTGCTTCATGCT |
| SEQ ID NO: 1631 | GCCTCCCGTAGGAGT |
| | SEQ ID NO: 1632 |
| GCTTGGAT | GCCTCCCTCGCGCCATCAGGCTTGGATCATGCT |
| SEQ ID NO: 1633 | GCCTCCCGTAGGAGT |
| | SEQ ID NO: 1634 |
| GCTTGGTA | GCCTCCCTCGCGCCATCAGGCTTGGTACATGCT |
| SEQ ID NO: 1635 | GCCTCCCGTAGGAGT |
| | SEQ ID NO: 1636 |
| GGAACCAA | GCCTCCCTCGCGCCATCAGGGAACCAACATGCT |
| SEQ ID NO: 1637 | GCCTCCCGTAGGAGT |
| | SEQ ID NO: 1638 |
| GGAACCTT | GCCTCCCTCGCGCCATCAGGGAACCTTCATGCT |
| SEQ ID NO: 1639 | GCCTCCCGTAGGAGT |
| | SEQ ID NO: 1640 |
| GGAACGAT | GCCTCCCTCGCGCCATCAGGGAACGATCATGCT |
| SEQ ID NO: 1641 | GCCTCCCGTAGGAGT |
| | SEQ ID NO: 1642 |
| GGAACGTA | GCCTCCCTCGCGCCATCAGGGAACGTACATGCT |
| SEQ ID NO: 1643 | GCCTCCCGTAGGAGT |
| | SEQ ID NO: 1644 |
| GGAAGCAT | GCCTCCCTCGCGCCATCAGGGAAGCATCATGCT |
| SEQ ID NO: 1645 | GCCTCCCGTAGGAGT |
| | SEQ ID NO: 1646 |
| GGAAGCTA | GCCTCCCTCGCGCCATCAGGGAAGCTACATGCT |
| SEQ ID NO: 1647 | GCCTCCCGTAGGAGT |
| | SEQ ID NO: 1648 |
| GGAAGGAA | GCCTCCCTCGCGCCATCAGGGAAGGAACATGCT |
| SEQ ID NO: 1649 | GCCTCCCGTAGGAGT |
| | SEQ ID NO: 1650 |
| GGAAGGTT | GCCTCCCTCGCGCCATCAGGGAAGGTTCATGCT |
| SEQ ID NO: 1651 | GCCTCCCGTAGGAGT |
| | SEQ ID NO: 1652 |
| GGAATACG | GCCTCCCTCGCGCCATCAGGGAATACGCATGCT |
| SEQ ID NO: 1653 | GCCTCCCGTAGGAGT |
| | SEQ ID NO: 1654 |
| GGAATAGC | GCCTCCCTCGCGCCATCAGGGAATAGCCATGCT |
| SEQ ID NO: 1655 | GCCTCCCGTAGGAGT |
| | SEQ ID NO: 1656 |
| GGAATTCC | GCCTCCCTCGCGCCATCAGGGAATTCCCATGCT |
| SEQ ID NO: 1657 | GCCTCCCGTAGGAGT |
| | SEQ ID NO: 1658 |
| GGAATTGG | GCCTCCCTCGCGCCATCAGGGAATTGGCATGCT |
| SEQ ID NO: 1659 | GCCTCCCGTAGGAGT |
| | SEQ ID NO: 1660 |
| GGATAACG | GCCTCCCTCGCGCCATCAGGGATAACGCATGCT |
| SEQ ID NO: 1661 | GCCTCCCGTAGGAGT |
| | SEQ ID NO: 1662 |
| GGATAAGC | GCCTCCCTCGCGCCATCAGGGATAAGCCATGCT |
| SEQ ID NO: 1663 | GCCTCCCGTAGGAGT |
| | SEQ ID NO: 1664 |
| GGATATCC | GCCTCCCTCGCGCCATCAGGGATATCCCATGCT |
| SEQ ID NO: 1665 | GCCTCCCGTAGGAGT |
| | SEQ ID NO: 1666 |
| GGATATGG | GCCTCCCTCGCGCCATCAGGGATATGGCATGCT |
| SEQ ID NO: 1667 | GCCTCCCGTAGGAGT |
| | SEQ ID NO: 1668 |
| GGATCCAT | GCCTCCCTCGCGCCATCAGGGATCCATCATGCT |
| SEQ ID NO: 1669 | GCCTCCCGTAGGAGT |
| | SEQ ID NO: 1670 |
| GGATCCTA | GCCTCCCTCGCGCCATCAGGGATCCTACATGCT |
| SEQ ID NO: 1671 | GCCTCCCGTAGGAGT |
| | SEQ ID NO: 1672 |
| GGATCGAA | GCCTCCCTCGCGCCATCAGGGATCGAACATGCT |
| SEQ ID NO: 1673 | GCCTCCCGTAGGAGT |
| | SEQ ID NO: 1674 |
| GGATCGTT | GCCTCCCTCGCGCCATCAGGGATCGTTCATGCT |
| SEQ ID NO: 1675 | GCCTCCCGTAGGAGT |
| | SEQ ID NO: 1676 |
| GGATGCAA | GCCTCCCTCGCGCCATCAGGGATGCAACATGCT |
| SEQ ID NO: 1677 | GCCTCCCGTAGGAGT |
| | SEQ ID NO: 1678 |
| GGATGCTT | GCCTCCCTCGCGCCATCAGGGATGCTTCATGCT |
| SEQ ID NO: 1679 | GCCTCCCGTAGGAGT |
| | SEQ ID NO: 1680 |
| GGATGGAT | GCCTCCCTCGCGCCATCAGGGATGGATCATGCT |
| SEQ ID NO: 1681 | GCCTCCCGTAGGAGT |
| | SEQ ID NO: 1682 |
| GGATGGTA | GCCTCCCTCGCGCCATCAGGGATGGTACATGCT |
| SEQ ID NO: 1683 | GCCTCCCGTAGGAGT |
| | SEQ ID NO: 1684 |
| GGATTACC | GCCTCCCTCGCGCCATCAGGGATTACCCATGCT |
| SEQ ID NO: 1685 | GCCTCCCGTAGGAGT |
| | SEQ ID NO: 1686 |
| GGATTAGG | GCCTCCCTCGCGCCATCAGGGATTAGGCATGCT |
| SEQ ID NO: 1687 | GCCTCCCGTAGGAGT |
| | SEQ ID NO: 1688 |
| GGCCAATT | GCCTCCCTCGCGCCATCAGGGCCAATTCATGCT |
| SEQ ID NO: 1689 | GCCTCCCGTAGGAGT |
| | SEQ ID NO: 1690 |
| GGCCATAT | GCCTCCCTCGCGCCATCAGGGCCATATCATGCT |
| SEQ ID NO: 1691 | GCCTCCCGTAGGAGT |
| | SEQ ID NO: 1692 |
| GGCCATTA | GCCTCCCTCGCGCCATCAGGGCCATTACATGCT |
| SEQ ID NO: 1693 | GCCTCCCGTAGGAGT |
| | SEQ ID NO: 1694 |
| GGCCTAAT | GCCTCCCTCGCGCCATCAGGGCCTAATCATGCT |
| SEQ ID NO: 1695 | GCCTCCCGTAGGAGT |
| | SEQ ID NO: 1696 |
| GGCCTATA | GCCTCCCTCGCGCCATCAGGGCCTATACATGCT |
| SEQ ID NO: 1697 | GCCTCCCGTAGGAGT |
| | SEQ ID NO: 1698 |
| GGCCTTAA | GCCTCCCTCGCGCCATCAGGGCCTTAACATGCT |
| SEQ ID NO: 1699 | GCCTCCCGTAGGAGT |
| | SEQ ID NO: 1700 |
| GGCGAATA | GCCTCCCTCGCGCCATCAGGGCGAATACATGCT |
| SEQ ID NO: 1701 | GCCTCCCGTAGGAGT |
| | SEQ ID NO: 1702 |
| GGCGATAA | GCCTCCCTCGCGCCATCAGGGCGATAACATGCT |
| SEQ ID NO: 1703 | GCCTCCCGTAGGAGT |
| | SEQ ID NO: 1704 |
| GGCGTATT | GCCTCCCTCGCGCCATCAGGGCGTATTCATGCT |
| SEQ ID NO: 1705 | GCCTCCCGTAGGAGT |
| | SEQ ID NO: 1706 |
| GGCGTTAT | GCCTCCCTCGCGCCATCAGGGCGTTATCATGCT |
| SEQ ID NO: 1707 | GCCTCCCGTAGGAGT |
| | SEQ ID NO: 1708 |
| GGTAATCC | GCCTCCCTCGCGCCATCAGGGTAATCCCATGCT |
| SEQ ID NO: 1709 | GCCTCCCGTAGGAGT |
| | SEQ ID NO: 1710 |
| GGTAATGG | GCCTCCCTCGCGCCATCAGGGTAATGGCATGCT |
| SEQ ID NO: 1711 | GCCTCCCGTAGGAGT |
| | SEQ ID NO: 1712 |
| GGTACCAT | GCCTCCCTCGCGCCATCAGGGTACCATCATGCT |
| SEQ ID NO: 1713 | GCCTCCCGTAGGAGT |
| | SEQ ID NO: 1714 |
| GGTACCTA | GCCTCCCTCGCGCCATCAGGGTACCTACATGCT |
| SEQ ID NO: 1715 | GCCTCCCGTAGGAGT |
| | SEQ ID NO: 1716 |
| GGTACGAA | GCCTCCCTCGCGCCATCAGGGTACGAACATGCT |
| SEQ ID NO: 1717 | GCCTCCCGTAGGAGT |
| | SEQ ID NO: 1718 |
| GGTACGTT | GCCTCCCTCGCGCCATCAGGGTACGTTCATGCT |
| SEQ ID NO: 1719 | GCCTCCCGTAGGAGT |
| | SEQ ID NO: 1720 |
| GGTAGCAA | GCCTCCCTCGCGCCATCAGGGTAGCAACATGCT |
| SEQ ID NO: 1721 | GCCTCCCGTAGGAGT |
| | SEQ ID NO: 1722 |
| GGTAGCTT | GCCTCCCTCGCGCCATCAGGGTAGCTTCATGCT |
| SEQ ID NO: 1723 | GCCTCCCGTAGGAGT |
| | SEQ ID NO: 1724 |
| GGTAGGAT | GCCTCCCTCGCGCCATCAGGGTAGGATCATGCT |
| SEQ ID NO: 1725 | GCCTCCCGTAGGAGT |
| | SEQ ID NO: 1726 |
| GGTAGGTA | GCCTCCCTCGCGCCATCAGGGTAGGTACATGCT |
| SEQ ID NO: 1727 | GCCTCCCGTAGGAGT |
| | SEQ ID NO: 1728 |
| GGTATACC | GCCTCCCTCGCGCCATCAGGGTATACCCATGCT |
| SEQ ID NO: 1729 | GCCTCCCGTAGGAGT |
| | SEQ ID NO: 1730 |
| GGTATAGG | GCCTCCCTCGCGCCATCAGGGTATAGGCATGCT |
| SEQ ID NO: 1731 | GCCTCCCGTAGGAGT |
| | SEQ ID NO: 1732 |
| GGTATTCG | GCCTCCCTCGCGCCATCAGGGTATTCGCATGCT |
| SEQ ID NO: 1733 | GCCTCCCGTAGGAGT |
| | SEQ ID NO: 1734 |
| GGTATTGC | GCCTCCCTCGCGCCATCAGGGTATTGCCATGCT |
| SEQ ID NO: 1735 | GCCTCCCGTAGGAGT |
| | SEQ ID NO: 1736 |
| GGTTAACC | GCCTCCCTCGCGCCATCAGGGTTAACCCATGCT |
| SEQ ID NO: 1737 | GCCTCCCGTAGGAGT |
| | SEQ ID NO: 1738 |
| GGTTAAGG | GCCTCCCTCGCGCCATCAGGGTTAAGGCATGCT |
| SEQ ID NO: 1739 | GCCTCCCGTAGGAGT |
| | SEQ ID NO: 1740 |
| GGTTATCG | GCCTCCCTCGCGCCATCAGGGTTATCGCATGCT |
| SEQ ID NO: 1741 | GCCTCCCGTAGGAGT |
| | SEQ ID NO: 1742 |
| GGTTATGC | GCCTCCCTCGCGCCATCAGGGTTATGCCATGCT |
| SEQ ID NO: 1743 | GCCTCCCGTAGGAGT |
| | SEQ ID NO: 1744 |
| GGTTCCAA | GCCTCCCTCGCGCCATCAGGGTTCCAACATGCT |
| SEQ ID NO: 1745 | GCCTCCCGTAGGAGT |
| | SEQ ID NO: 1746 |
| GGTTCCTT | GCCTCCCTCGCGCCATCAGGGTTCCTTCATGCT |
| SEQ ID NO: 1747 | GCCTCCCGTAGGAGT |
| | SEQ ID NO: 1748 |
| GGTTCGAT | GCCTCCCTCGCGCCATCAGGGTTCGATCATGCT |
| SEQ ID NO: 1749 | GCCTCCCGTAGGAGT |
| | SEQ ID NO: 1750 |
| GGTTCGTA | GCCTCCCTCGCGCCATCAGGGTTCGTACATGCT |
| SEQ ID NO: 1751 | GCCTCCCGTAGGAGT |
| | SEQ ID NO: 1752 |
| GGTTGCAT | GCCTCCCTCGCGCCATCAGGGTTGCATCATGCT |
| SEQ ID NO: 1753 | GCCTCCCGTAGGAGT |
| | SEQ ID NO: 1754 |
| GGTTGCTA | GCCTCCCTCGCGCCATCAGGGTTGCTACATGCT |
| SEQ ID NO: 1755 | GCCTCCCGTAGGAGT |
| | SEQ ID NO: 1756 |
| GGTTGGAA | GCCTCCCTCGCGCCATCAGGGTTGGAACATGCT |
| SEQ ID NO: 1757 | GCCTCCCGTAGGAGT |
| | SEQ ID NO: 1758 |
| GGTTGGTT | GCCTCCCTCGCGCCATCAGGGTTGGTTCATGCT |
| SEQ ID NO: 1759 | GCCTCCCGTAGGAGT |
| | SEQ ID NO: 1760 |
| GTACACCA | GCCTCCCTCGCGCCATCAGGTACACCACATGCT |
| SEQ ID NO: 1761 | GCCTCCCGTAGGAGT |
| | SEQ ID NO: 1762 |
| GTACACGT | GCCTCCCTCGCGCCATCAGGTACACGTCATGCT |
| SEQ ID NO: 1763 | GCCTCCCGTAGGAGT |
| | SEQ ID NO: 1764 |
| GTACAGCT | GCCTCCCTCGCGCCATCAGGTACAGCTCATGCT |
| SEQ ID NO: 1765 | GCCTCCCGTAGGAGT |
| | SEQ ID NO: 1766 |
| GTACAGGA | GCCTCCCTCGCGCCATCAGGTACAGGACATGCT |
| SEQ ID NO: 1767 | GCCTCCCGTAGGAGT |
| | SEQ ID NO: 1768 |
| GTACCAAC | GCCTCCCTCGCGCCATCAGGTACCAACCATGCT |
| SEQ ID NO: 1769 | GCCTCCCGTAGGAGT |
| | SEQ ID NO: 1770 |
| GTACCATG | GCCTCCCTCGCGCCATCAGGTACCATGCATGCT |
| SEQ ID NO: 1771 | GCCTCCCGTAGGAGT |
| | SEQ ID NO: 1772 |
| GTACCTAG | GCCTCCCTCGCGCCATCAGGTACCTAGCATGCT |
| SEQ ID NO: 1773 | GCCTCCCGTAGGAGT |
| | SEQ ID NO: 1774 |
| GTACCTTC | GCCTCCCTCGCGCCATCAGGTACCTTCCATGCT |
| SEQ ID NO: 1775 | GCCTCCCGTAGGAGT |
| | SEQ ID NO: 1776 |
| GTACGAAG | GCCTCCCTCGCGCCATCAGGTACGAAGCATGCT |
| SEQ ID NO: 1777 | GCCTCCCGTAGGAGT |
| | SEQ ID NO: 1778 |
| GTACGATC | GCCTCCCTCGCGCCATCAGGTACGATCCATGCT |
| SEQ ID NO: 1779 | GCCTCCCGTAGGAGT |
| | SEQ ID NO: 1780 |
| GTACGTAC | GCCTCCCTCGCGCCATCAGGTACGTACCATGCT |
| SEQ ID NO: 1781 | GCCTCCCGTAGGAGT |
| | SEQ ID NO: 1782 |
| GTACGTTG | GCCTCCCTCGCGCCATCAGGTACGTTGCATGCT |
| SEQ ID NO: 1783 | GCCTCCCGTAGGAGT |
| | SEQ ID NO: 1784 |
| GTACTCCT | GCCTCCCTCGCGCCATCAGGTACTCCTCATGCT |
| SEQ ID NO: 1785 | GCCTCCCGTAGGAGT |
| | SEQ ID NO: 1786 |
| GTACTCGA | GCCTCCCTCGCGCCATCAGGTACTCGACATGCT |
| SEQ ID NO: 1787 | GCCTCCCGTAGGAGT |
| | SEQ ID NO: 1788 |
| GTACTGCA | GCCTCCCTCGCGCCATCAGGTACTGCACATGCT |
| SEQ ID NO: 1789 | GCCTCCCGTAGGAGT |
| | SEQ ID NO: 1790 |
| GTACTGGT | GCCTCCCTCGCGCCATCAGGTACTGGTCATGCT |
| SEQ ID NO: 1791 | GCCTCCCGTAGGAGT |
| | SEQ ID NO: 1792 |
| GTAGACCT | GCCTCCCTCGCGCCATCAGGTAGACCTCATGCT |
| SEQ ID NO: 1793 | GCCTCCCGTAGGAGT |
| | SEQ ID NO: 1794 |
| GTAGACGA | GCCTCCCTCGCGCCATCAGGTAGACGACATGCT |
| SEQ ID NO: 1795 | GCCTCCCGTAGGAGT |
| | SEQ ID NO: 1796 |
| GTAGAGCA | GCCTCCCTCGCGCCATCAGGTAGAGCACATGCT |
| SEQ ID NO: 1797 | GCCTCCCGTAGGAGT |
| | SEQ ID NO: 1798 |
| GTAGAGGT | GCCTCCCTCGCGCCATCAGGTAGAGGTCATGCT |
| SEQ ID NO: 1799 | GCCTCCCGTAGGAGT |
| | SEQ ID NO: 1800 |
| GTAGCAAG | GCCTCCCTCGCGCCATCAGGTAGCAAGCATGCT |
| SEQ ID NO: 1801 | GCCTCCCGTAGGAGT |
| | SEQ ID NO: 1802 |
| GTAGCATC | GCCTCCCTCGCGCCATCAGGTAGCATCCATGCT |
| SEQ ID NO: 1803 | GCCTCCCGTAGGAGT |
| | SEQ ID NO: 1804 |
| GTAGCTAC | GCCTCCCTCGCGCCATCAGGTAGCTACCATGCT |
| SEQ ID NO: 1805 | GCCTCCCGTAGGAGT |
| | SEQ ID NO: 1806 |
| GTAGCTTG | GCCTCCCTCGCGCCATCAGGTAGCTTGCATGCT |
| SEQ ID NO: 1807 | GCCTCCCGTAGGAGT |
| | SEQ ID NO: 1808 |
| GTAGGAAC | GCCTCCCTCGCGCCATCAGGTAGGAACCATGCT |
| SEQ ID NO: 1809 | GCCTCCCGTAGGAGT |
| | SEQ ID NO: 1810 |
| GTAGGATG | GCCTCCCTCGCGCCATCAGGTAGGATGCATGCT |
| SEQ ID NO: 1811 | GCCTCCCGTAGGAGT |
| | SEQ ID NO: 1812 |
| GTAGGTAG | GCCTCCCTCGCGCCATCAGGTAGGTAGCATGCT |
| SEQ ID NO: 1813 | GCCTCCCGTAGGAGT |
| | SEQ ID NO: 1814 |
| GTAGGTTC | GCCTCCCTCGCGCCATCAGGTAGGTTCCATGCT |
| SEQ ID NO: 1815 | GCCTCCCGTAGGAGT |
| | SEQ ID NO: 1816 |
| GTAGTCCA | GCCTCCCTCGCGCCATCAGGTAGTCCACATGCT |
| SEQ ID NO: 1817 | GCCTCCCGTAGGAGT |
| | SEQ ID NO: 1818 |
| GTAGTCGT | GCCTCCCTCGCGCCATCAGGTAGTCGTCATGCT |
| SEQ ID NO: 1819 | GCCTCCCGTAGGAGT |
| | SEQ ID NO: 1820 |
| GTAGTGCT | GCCTCCCTCGCGCCATCAGGTAGTGCTCATGCT |
| SEQ ID NO: 1821 | GCCTCCCGTAGGAGT |
| | SEQ ID NO: 1822 |
| GTAGTGGA | GCCTCCCTCGCGCCATCAGGTAGTGGACATGCT |
| SEQ ID NO: 1823 | GCCTCCCGTAGGAGT |
| | SEQ ID NO: 1824 |
| GTCAACAC | GCCTCCCTCGCGCCATCAGGTCAACACCATGCT |
| SEQ ID NO: 1825 | GCCTCCCGTAGGAGT |
| | SEQ ID NO: 1826 |
| GTCAACTG | GCCTCCCTCGCGCCATCAGGTCAACTGCATGCT |
| SEQ ID NO: 1827 | GCCTCCCGTAGGAGT |
| | SEQ ID NO: 1828 |
| GTCAAGAG | GCCTCCCTCGCGCCATCAGGTCAAGAGCATGCT |
| SEQ ID NO: 1829 | GCCTCCCGTAGGAGT |
| | SEQ ID NO: 1830 |
| GTCAAGTC | GCCTCCCTCGCGCCATCAGGTCAAGTCCATGCT |
| SEQ ID NO: 1831 | GCCTCCCGTAGGAGT |
| | SEQ ID NO: 1832 |
| GTCACACA | GCCTCCCTCGCGCCATCAGGTCACACACATGCT |
| SEQ ID NO: 1833 | GCCTCCCGTAGGAGT |
| | SEQ ID NO: 1834 |
| GTCACAGT | GCCTCCCTCGCGCCATCAGGTCACAGTCATGCT |
| SEQ ID NO: 1835 | GCCTCCCGTAGGAGT |
| | SEQ ID NO: 1836 |
| GTCACTCT | GCCTCCCTCGCGCCATCAGGTCACTCTCATGCT |
| SEQ ID NO: 1837 | GCCTCCCGTAGGAGT |
| | SEQ ID NO: 1838 |
| GTCACTGA | GCCTCCCTCGCGCCATCAGGTCACTGACATGCT |
| SEQ ID NO: 1839 | GCCTCCCGTAGGAGT |
| | SEQ ID NO: 1840 |
| GTCAGACT | GCCTCCCTCGCGCCATCAGGTCAGACTCATGCT |
| SEQ ID NO: 1841 | GCCTCCCGTAGGAGT |
| | SEQ ID NO: 1842 |
| GTCAGAGA | GCCTCCCTCGCGCCATCAGGTCAGAGACATGCT |
| SEQ ID NO: 1843 | GCCTCCCGTAGGAGT |
| | SEQ ID NO: 1844 |
| GTCAGTCA | GCCTCCCTCGCGCCATCAGGTCAGTCACATGCT |
| SEQ ID NO: 1845 | GCCTCCCGTAGGAGT |
| | SEQ ID NO: 1846 |
| GTCAGTGT | GCCTCCCTCGCGCCATCAGGTCAGTGTCATGCT |
| SEQ ID NO: 1847 | GCCTCCCGTAGGAGT |
| | SEQ ID NO: 1848 |
| GTCATCAG | GCCTCCCTCGCGCCATCAGGTCATCAGCATGCT |
| SEQ ID NO: 1849 | GCCTCCCGTAGGAGT |
| | SEQ ID NO: 1850 |
| GTCATCTC | GCCTCCCTCGCGCCATCAGGTCATCTCCATGCT |
| SEQ ID NO: 1851 | GCCTCCCGTAGGAGT |
| | SEQ ID NO: 1852 |
| GTCATGAC | GCCTCCCTCGCGCCATCAGGTCATGACCATGCT |
| SEQ ID NO: 1853 | GCCTCCCGTAGGAGT |
| | SEQ ID NO: 1854 |
| GTCATGTG | GCCTCCCTCGCGCCATCAGGTCATGTGCATGCT |
| SEQ ID NO: 1855 | GCCTCCCGTAGGAGT |
| | SEQ ID NO: 1856 |
| GTCTACAG | GCCTCCCTCGCGCCATCAGGTCTACAGCATGCT |
| SEQ ID NO: 1857 | GCCTCCCGTAGGAGT |
| | SEQ ID NO: 1858 |
| GTCTACTC | GCCTCCCTCGCGCCATCAGGTCTACTCCATGCT |
| SEQ ID NO: 1859 | GCCTCCCGTAGGAGT |
| | SEQ ID NO: 1860 |
| GTCTAGAC | GCCTCCCTCGCGCCATCAGGTCTAGACCATGCT |
| SEQ ID NO: 1861 | GCCTCCCGTAGGAGT |
| | SEQ ID NO: 1862 |
| GTCTAGTG | GCCTCCCTCGCGCCATCAGGTCTAGTGCATGCT |
| SEQ ID NO: 1863 | GCCTCCCGTAGGAGT |
| | SEQ ID NO: 1864 |
| GTCTCACT | GCCTCCCTCGCGCCATCAGGTCTCACTCATGCT |
| SEQ ID NO: 1865 | GCCTCCCGTAGGAGT |
| | SEQ ID NO: 1866 |
| GTCTCAGA | GCCTCCCTCGCGCCATCAGGTCTCAGACATGCT |
| SEQ ID NO: 1867 | GCCTCCCGTAGGAGT |
| | SEQ ID NO: 1868 |
| GTCTCTCA | GCCTCCCTCGCGCCATCAGGTCTCTCACATGCT |
| SEQ ID NO: 1869 | GCCTCCCGTAGGAGT |
| | SEQ ID NO: 1870 |
| GTCTCTGT | GCCTCCCTCGCGCCATCAGGTCTCTGTCATGCT |
| SEQ ID NO: 1871 | GCCTCCCGTAGGAGT |
| | SEQ ID NO: 1872 |
| GTCTGACA | GCCTCCCTCGCGCCATCAGGTCTGACACATGCT |
| SEQ ID NO: 1873 | GCCTCCCGTAGGAGT |
| | SEQ ID NO: 1874 |
| GTCTGAGT | GCCTCCCTCGCGCCATCAGGTCTGAGTCATGCT |
| SEQ ID NO: 1875 | GCCTCCCGTAGGAGT |
| | SEQ ID NO: 1876 |
| GTCTGTCT | GCCTCCCTCGCGCCATCAGGTCTGTCTCATGCT |
| SEQ ID NO: 1877 | GCCTCCCGTAGGAGT |
| | SEQ ID NO: 1878 |
| GTCTGTGA | GCCTCCCTCGCGCCATCAGGTCTGTGACATGCT |
| SEQ ID NO: 1879 | GCCTCCCGTAGGAGT |
| | SEQ ID NO: 1880 |
| GTCTTCAC | GCCTCCCTCGCGCCATCAGGTCTTCACCATGCT |
| SEQ ID NO: 1881 | GCCTCCCGTAGGAGT |
| | SEQ ID NO: 1882 |
| GTCTTCTG | GCCTCCCTCGCGCCATCAGGTCTTCTGCATGCT |
| SEQ ID NO: 1883 | GCCTCCCGTAGGAGT |
| | SEQ ID NO: 1884 |
| GTCTTGAG | GCCTCCCTCGCGCCATCAGGTCTTGAGCATGCT |
| SEQ ID NO: 1885 | GCCTCCCGTAGGAGT |
| | SEQ ID NO: 1886 |
| GTCTTGTC | GCCTCCCTCGCGCCATCAGGTCTTGTCCATGCT |
| SEQ ID NO: 1887 | GCCTCCCGTAGGAGT |
| | SEQ ID NO: 1888 |
| GTGAACAG | GCCTCCCTCGCGCCATCAGGTGAACAGCATGCT |
| SEQ ID NO: 1889 | GCCTCCCGTAGGAGT |
| | SEQ ID NO: 1890 |
| GTGAACTC | GCCTCCCTCGCGCCATCAGGTGAACTCCATGCT |
| SEQ ID NO: 1891 | GCCTCCCGTAGGAGT |
| | SEQ ID NO: 1892 |
| GTGAAGAC | GCCTCCCTCGCGCCATCAGGTGAAGACCATGCT |
| SEQ ID NO: 1893 | GCCTCCCGTAGGAGT |
| | SEQ ID NO: 1894 |
| GTGAAGTG | GCCTCCCTCGCGCCATCAGGTGAAGTGCATGCT |
| SEQ ID NO: 1895 | GCCTCCCGTAGGAGT |
| | SEQ ID NO: 1896 |
| GTGACACT | GCCTCCCTCGCGCCATCAGGTGACACTCATGCT |
| SEQ ID NO: 1897 | GCCTCCCGTAGGAGT |
| | SEQ ID NO: 1898 |
| GTGACAGA | GCCTCCCTCGCGCCATCAGGTGACAGACATGCT |
| SEQ ID NO: 1899 | GCCTCCCGTAGGAGT |
| | SEQ ID NO: 1900 |
| GTGACTCA | GCCTCCCTCGCGCCATCAGGTGACTCACATGCT |
| SEQ ID NO: 1901 | GCCTCCCGTAGGAGT |
| | SEQ ID NO: 1902 |
| GTGACTGT | GCCTCCCTCGCGCCATCAGGTGACTGTCATGCT |
| SEQ ID NO: 1903 | GCCTCCCGTAGGAGT |
| | SEQ ID NO: 1904 |
| GTGAGACA | GCCTCCCTCGCGCCATCAGGTGAGACACATGCT |
| SEQ ID NO: 1905 | GCCTCCCGTAGGAGT |
| | SEQ ID NO: 1906 |
| GTGAGAGT | GCCTCCCTCGCGCCATCAGGTGAGAGTCATGCT |
| SEQ ID NO: 1907 | GCCTCCCGTAGGAGT |
| | SEQ ID NO: 1908 |
| GTGAGTCT | GCCTCCCTCGCGCCATCAGGTGAGTCTCATGCT |
| SEQ ID NO: 1909 | GCCTCCCGTAGGAGT |
| | SEQ ID NO: 1910 |
| GTGAGTGA | GCCTCCCTCGCGCCATCAGGTGAGTGACATGCT |
| SEQ ID NO: 1911 | GCCTCCCGTAGGAGT |
| | SEQ ID NO: 1912 |
| GTGATCAC | GCCTCCCTCGCGCCATCAGGTGATCACCATGCT |
| SEQ ID NO: 1913 | GCCTCCCGTAGGAGT |
| | SEQ ID NO: 1914 |
| GTGATCTG | GCCTCCCTCGCGCCATCAGGTGATCTGCATGCT |
| SEQ ID NO: 1915 | GCCTCCCGTAGGAGT |
| | SEQ ID NO: 1916 |
| GTGATGAG | GCCTCCCTCGCGCCATCAGGTGATGAGCATGCT |
| SEQ ID NO: 1917 | GCCTCCCGTAGGAGT |
| | SEQ ID NO: 1918 |
| GTGATGTC | GCCTCCCTCGCGCCATCAGGTGATGTCCATGCT |
| SEQ ID NO: 1919 | GCCTCCCGTAGGAGT |
| | SEQ ID NO: 1920 |
| GTGTACAC | GCCTCCCTCGCGCCATCAGGTGTACACCATGCT |
| SEQ ID NO: 1921 | GCCTCCCGTAGGAGT |
| | SEQ ID NO: 1922 |
| GTGTACTG | GCCTCCCTCGCGCCATCAGGTGTACTGCATGCT |
| SEQ ID NO: 1923 | GCCTCCCGTAGGAGT |
| | SEQ ID NO: 1924 |
| GTGTAGAG | GCCTCCCTCGCGCCATCAGGTGTAGAGCATGCT |
| SEQ ID NO: 1925 | GCCTCCCGTAGGAGT |
| | SEQ ID NO: 1926 |
| GTGTAGTC | GCCTCCCTCGCGCCATCAGGTGTAGTCCATGCT |
| SEQ ID NO: 1927 | GCCTCCCGTAGGAGT |
| | SEQ ID NO: 1928 |
| GTGTCACA | GCCTCCCTCGCGCCATCAGGTGTCACACATGCT |
| SEQ ID NO: 1929 | GCCTCCCGTAGGAGT |
| | SEQ ID NO: 1930 |
| GTGTCAGT | GCCTCCCTCGCGCCATCAGGTGTCAGTCATGCT |
| SEQ ID NO: 1931 | GCCTCCCGTAGGAGT |
| | SEQ ID NO: 1932 |
| GTGTCTCT | GCCTCCCTCGCGCCATCAGGTGTCTCTCATGCT |
| SEQ ID NO: 1933 | GCCTCCCGTAGGAGT |
| | SEQ ID NO: 1934 |
| GTGTCTGA | GCCTCCCTCGCGCCATCAGGTGTCTGACATGCT |
| SEQ ID NO: 1935 | GCCTCCCGTAGGAGT |
| | SEQ ID NO: 1936 |
| GTGTGACT | GCCTCCCTCGCGCCATCAGGTGTGACTCATGCT |
| SEQ ID NO: 1937 | GCCTCCCGTAGGAGT |
| | SEQ ID NO: 1938 |
| GTGTGAGA | GCCTCCCTCGCGCCATCAGGTGTGAGACATGCT |
| SEQ ID NO: 1939 | GCCTCCCGTAGGAGT |
| | SEQ ID NO: 1940 |
| GTGTGTCA | GCCTCCCTCGCGCCATCAGGTGTGTCACATGCT |
| SEQ ID NO: 1941 | GCCTCCCGTAGGAGT |
| | SEQ ID NO: 1942 |
| GTGTGTGT | GCCTCCCTCGCGCCATCAGGTGTGTGTCATGCT |
| SEQ ID NO: 1943 | GCCTCCCGTAGGAGT |
| | SEQ ID NO: 1944 |
| GTGTTCAG | GCCTCCCTCGCGCCATCAGGTGTTCAGCATGCT |
| SEQ ID NO: 1945 | GCCTCCCGTAGGAGT |
| | SEQ ID NO: 1946 |
| GTGTTCTC | GCCTCCCTCGCGCCATCAGGTGTTCTCCATGCT |
| SEQ ID NO: 1947 | GCCTCCCGTAGGAGT |
| | SEQ ID NO: 1948 |
| GTGTTGAC | GCCTCCCTCGCGCCATCAGGTGTTGACCATGCT |
| SEQ ID NO: 1949 | GCCTCCCGTAGGAGT |
| | SEQ ID NO: 1950 |
| GTGTTGTG | GCCTCCCTCGCGCCATCAGGTGTTGTGCATGCT |
| SEQ ID NO: 1951 | GCCTCCCGTAGGAGT |
| | SEQ ID NO: 1952 |
| GTTCACCT | GCCTCCCTCGCGCCATCAGGTTCACCTCATGCT |
| SEQ ID NO: 1953 | GCCTCCCGTAGGAGT |
| | SEQ ID NO: 1954 |
| GTTCACGA | GCCTCCCTCGCGCCATCAGGTTCACGACATGCT |
| SEQ ID NO: 1955 | GCCTCCCGTAGGAGT |
| | SEQ ID NO: 1956 |
| GTTCAGCA | GCCTCCCTCGCGCCATCAGGTTCAGCACATGCT |
| SEQ ID NO: 1957 | GCCTCCCGTAGGAGT |
| | SEQ ID NO: 1958 |
| GTTCAGGT | GCCTCCCTCGCGCCATCAGGTTCAGGTCATGCT |
| SEQ ID NO: 1959 | GCCTCCCGTAGGAGT |
| | SEQ ID NO: 1960 |
| GTTCCAAG | GCCTCCCTCGCGCCATCAGGTTCCAAGCATGCT |
| SEQ ID NO: 1961 | GCCTCCCGTAGGAGT |
| | SEQ ID NO: 1962 |
| GTTCCATC | GCCTCCCTCGCGCCATCAGGTTCCATCCATGCT |
| SEQ ID NO: 1963 | GCCTCCCGTAGGAGT |
| | SEQ ID NO: 1964 |
| GTTCCTAC | GCCTCCCTCGCGCCATCAGGTTCCTACCATGCT |
| SEQ ID NO: 1965 | GCCTCCCGTAGGAGT |
| | SEQ ID NO: 1966 |
| GTTCCTTG | GCCTCCCTCGCGCCATCAGGTTCCTTGCATGCT |
| SEQ ID NO: 1967 | GCCTCCCGTAGGAGT |
| | SEQ ID NO: 1968 |
| GTTCGAAC | GCCTCCCTCGCGCCATCAGGTTCGAACCATGCT |
| SEQ ID NO: 1969 | GCCTCCCGTAGGAGT |
| | SEQ ID NO: 1970 |
| GTTCGATG | GCCTCCCTCGCGCCATCAGGTTCGATGCATGCT |
| SEQ ID NO: 1971 | GCCTCCCGTAGGAGT |
| | SEQ ID NO: 1972 |
| GTTCGTAG | GCCTCCCTCGCGCCATCAGGTTCGTAGCATGCT |
| SEQ ID NO: 1973 | GCCTCCCGTAGGAGT |
| | SEQ ID NO: 1974 |
| GTTCGTTC | GCCTCCCTCGCGCCATCAGGTTCGTTCCATGCT |
| SEQ ID NO: 1975 | GCCTCCCGTAGGAGT |
| | SEQ ID NO: 1976 |
| GTTCTCCA | GCCTCCCTCGCGCCATCAGGTTCTCCACATGCT |
| SEQ ID NO: 1977 | GCCTCCCGTAGGAGT |
| | SEQ ID NO: 1978 |
| GTTCTCGT | GCCTCCCTCGCGCCATCAGGTTCTCGTCATGCT |
| SEQ ID NO: 1979 | GCCTCCCGTAGGAGT |
| | SEQ ID NO: 1980 |
| GTTCTGCT | GCCTCCCTCGCGCCATCAGGTTCTGCTCATGCT |
| SEQ ID NO: 1981 | GCCTCCCGTAGGAGT |
| | SEQ ID NO: 1982 |
| GTTCTGGA | GCCTCCCTCGCGCCATCAGGTTCTGGACATGCT |
| SEQ ID NO: 1983 | GCCTCCCGTAGGAGT |
| | SEQ ID NO: 1984 |
| GTTGACCA | GCCTCCCTCGCGCCATCAGGTTGACCACATGCT |
| SEQ ID NO: 1985 | GCCTCCCGTAGGAGT |
| | SEQ ID NO: 1986 |
| GTTGACGT | GCCTCCCTCGCGCCATCAGGTTGACGTCATGCT |
| SEQ ID NO: 1987 | GCCTCCCGTAGGAGT |
| | SEQ ID NO: 1988 |
| GTTGAGCT | GCCTCCCTCGCGCCATCAGGTTGAGCTCATGCT |
| SEQ ID NO: 1989 | GCCTCCCGTAGGAGT |
| | SEQ ID NO: 1990 |
| GTTGAGGA | GCCTCCCTCGCGCCATCAGGTTGAGGACATGCT |
| SEQ ID NO: 1991 | GCCTCCCGTAGGAGT |
| | SEQ ID NO: 1992 |
| GTTGCAAC | GCCTCCCTCGCGCCATCAGGTTGCAACCATGCT |
| SEQ ID NO: 1993 | GCCTCCCGTAGGAGT |
| | SEQ ID NO: 1994 |
| GTTGCATG | GCCTCCCTCGCGCCATCAGGTTGCATGCATGCT |
| SEQ ID NO: 1995 | GCCTCCCGTAGGAGT |
| | SEQ ID NO: 1996 |
| GTTGCTAG | GCCTCCCTCGCGCCATCAGGTTGCTAGCATGCT |
| SEQ ID NO: 1997 | GCCTCCCGTAGGAGT |
| | SEQ ID NO: 1998 |
| GTTGCTTC | GCCTCCCTCGCGCCATCAGGTTGCTTCCATGCT |
| SEQ ID NO: 1999 | GCCTCCCGTAGGAGT |
| | SEQ ID NO: 2000 |
| GTTGGAAG | GCCTCCCTCGCGCCATCAGGTTGGAAGCATGCT |
| SEQ ID NO: 2001 | GCCTCCCGTAGGAGT |
| | SEQ ID NO: 2002 |
| GTTGGATC | GCCTCCCTCGCGCCATCAGGTTGGATCCATGCT |
| SEQ ID NO: 2003 | GCCTCCCGTAGGAGT |
| | SEQ ID NO: 2004 |
| GTTGGTAC | GCCTCCCTCGCGCCATCAGGTTGGTACCATGCT |
| SEQ ID NO: 2005 | GCCTCCCGTAGGAGT |
| | SEQ ID NO: 2006 |
| GTTGGTTG | GCCTCCCTCGCGCCATCAGGTTGGTTGCATGCT |
| SEQ ID NO: 2007 | GCCTCCCGTAGGAGT |
| | SEQ ID NO: 2008 |
| GTTGTCCT | GCCTCCCTCGCGCCATCAGGTTGTCCTCATGCT |
| SEQ ID NO: 2009 | GCCTCCCGTAGGAGT |
| | SEQ ID NO: 2010 |
| GTTGTCGA | GCCTCCCTCGCGCCATCAGGTTGTCGACATGCT |
| SEQ ID NO: 2011 | GCCTCCCGTAGGAGT |
| | SEQ ID NO: 2012 |
| GTTGTGCA | GCCTCCCTCGCGCCATCAGGTTGTGCACATGCT |
| SEQ ID NO: 2013 | GCCTCCCGTAGGAGT |
| | SEQ ID NO: 2014 |
| GTTGTGGT | GCCTCCCTCGCGCCATCAGGTTGTGGTCATGCT |
| SEQ ID NO: 2015 | GCCTCCCGTAGGAGT |
| | SEQ ID NO: 2016 |
| TAATCCGG | GCCTCCCTCGCGCCATCAGTAATCCGGCATGCT |
| SEQ ID NO: 2017 | GCCTCCCGTAGGAGT |
| | SEQ ID NO: 2018 |
| TAATCGCG | GCCTCCCTCGCGCCATCAGTAATCGCGCATGCT |
| SEQ ID NO: 2019 | GCCTCCCGTAGGAGT |
| | SEQ ID NO: 2020 |
| TAATCGGC | GCCTCCCTCGCGCCATCAGTAATCGGCCATGCT |
| SEQ ID NO: 2021 | GCCTCCCGTAGGAGT |
| | SEQ ID NO: 2022 |
| TAATGCCG | GCCTCCCTCGCGCCATCAGTAATGCCGCATGCT |
| SEQ ID NO: 2023 | GCCTCCCGTAGGAGT |
| | SEQ ID NO: 2034 |
| TAATGCGC | GCCTCCCTCGCGCCATCAGTAATGCGCCATGCT |
| SEQ ID NO: 2035 | GCCTCCCGTAGGAGT |
| | SEQ ID NO: 2036 |
| TAATGGCC | GCCTCCCTCGCGCCATCAGTAATGGCCCATGCT |
| SEQ ID NO: 2037 | GCCTCCCGTAGGAGT |
| | SEQ ID NO: 2038 |
| TACCAACG | GCCTCCCTCGCGCCATCAGTACCAACGCATGCT |
| SEQ ID NO: 2039 | GCCTCCCGTAGGAGT |
| | SEQ ID NO: 2040 |
| TACCAAGC | GCCTCCCTCGCGCCATCAGTACCAAGCCATGCT |
| SEQ ID NO: 2041 | GCCTCCCGTAGGAGT |
| | SEQ ID NO: 2042 |
| TACCATCC | GCCTCCCTCGCGCCATCAGTACCATCCCATGCT |
| SEQ ID NO: 2043 | GCCTCCCGTAGGAGT |
| | SEQ ID NO: 2044 |
| TACCATGG | GCCTCCCTCGCGCCATCAGTACCATGGCATGCT |
| SEQ ID NO: 2045 | GCCTCCCGTAGGAGT |
| | SEQ ID NO: 2046 |
| TACCGCAA | GCCTCCCTCGCGCCATCAGTACCGCAACATGCT |
| SEQ ID NO: 2047 | GCCTCCCGTAGGAGT |
| | SEQ ID NO: 2048 |
| TACCGCTT | GCCTCCCTCGCGCCATCAGTACCGCTTCATGCT |
| SEQ ID NO: 2049 | GCCTCCCGTAGGAGT |
| | SEQ ID NO: 2050 |
| TACCGGAT | GCCTCCCTCGCGCCATCAGTACCGGATCATGCT |
| SEQ ID NO: 2051 | GCCTCCCGTAGGAGT |
| | SEQ ID NO: 2052 |
| TACCGGTA | GCCTCCCTCGCGCCATCAGTACCGGTACATGCT |
| SEQ ID NO: 2053 | GCCTCCCGTAGGAGT |
| | SEQ ID NO: 2054 |
| TACCTACC | GCCTCCCTCGCGCCATCAGTACCTACCCATGCT |
| SEQ ID NO: 2055 | GCCTCCCGTAGGAGT |
| | SEQ ID NO: 2056 |
| TACCTAGG | GCCTCCCTCGCGCCATCAGTACCTAGGCATGCT |
| SEQ ID NO: 2057 | GCCTCCCGTAGGAGT |
| | SEQ ID NO: 2058 |
| TACCTTCG | GCCTCCCTCGCGCCATCAGTACCTTCGCATGCT |
| SEQ ID NO: 2059 | GCCTCCCGTAGGAGT |
| | SEQ ID NO: 2060 |
| TACCTTGC | GCCTCCCTCGCGCCATCAGTACCTTGCCATGCT |
| SEQ ID NO: 2061 | GCCTCCCGTAGGAGT |
| | SEQ ID NO: 2062 |
| TACGAACC | GCCTCCCTCGCGCCATCAGTACGAACCCATGCT |
| SEQ ID NO: 2063 | GCCTCCCGTAGGAGT |
| | SEQ ID NO: 2064 |
| TACGAAGG | GCCTCCCTCGCGCCATCAGTACGAAGGCATGCT |
| SEQ ID NO: 2065 | GCCTCCCGTAGGAGT |
| | SEQ ID NO: 2066 |
| TACGATCG | GCCTCCCTCGCGCCATCAGTACGATCGCATGCT |
| SEQ ID NO: 2067 | GCCTCCCGTAGGAGT |
| | SEQ ID NO: 2068 |
| TACGATGC | GCCTCCCTCGCGCCATCAGTACGATGCCATGCT |
| SEQ ID NO: 2069 | GCCTCCCGTAGGAGT |
| | SEQ ID NO: 2070 |
| TACGCCAA | GCCTCCCTCGCGCCATCAGTACGCCAACATGCT |
| SEQ ID NO: 2071 | GCCTCCCGTAGGAGT |
| | SEQ ID NO: 2072 |
| TACGCCTT | GCCTCCCTCGCGCCATCAGTACGCCTTCATGCT |
| SEQ ID NO: 2073 | GCCTCCCGTAGGAGT |
| | SEQ ID NO: 2074 |
| TACGCGAT | GCCTCCCTCGCGCCATCAGTACGCGATCATGCT |
| SEQ ID NO: 2075 | GCCTCCCGTAGGAGT |
| | SEQ ID NO: 2076 |
| TACGCGTA | GCCTCCCTCGCGCCATCAGTACGCGTACATGCT |
| SEQ ID NO: 2077 | GCCTCCCGTAGGAGT |
| | SEQ ID NO: 2078 |
| TACGGCAT | GCCTCCCTCGCGCCATCAGTACGGCATCATGCT |
| SEQ ID NO: 2079 | GCCTCCCGTAGGAGT |
| | SEQ ID NO: 2080 |
| TACGGCTA | GCCTCCCTCGCGCCATCAGTACGGCTACATGCT |
| SEQ ID NO: 2081 | GCCTCCCGTAGGAGT |
| | SEQ ID NO: 2082 |
| TACGTACG | GCCTCCCTCGCGCCATCAGTACGTACGCATGCT |
| SEQ ID NO: 2083 | GCCTCCCGTAGGAGT |
| | SEQ ID NO: 2084 |
| TACGTAGC | GCCTCCCTCGCGCCATCAGTACGTAGCCATGCT |
| SEQ ID NO: 2085 | GCCTCCCGTAGGAGT |
| | SEQ ID NO: 2086 |
| TACGTTCC | GCCTCCCTCGCGCCATCAGTACGTTCCCATGCT |
| SEQ ID NO: 2087 | GCCTCCCGTAGGAGT |
| | SEQ ID NO: 2088 |
| TACGTTGG | GCCTCCCTCGCGCCATCAGTACGTTGGCATGCT |
| SEQ ID NO: 2089 | GCCTCCCGTAGGAGT |
| | SEQ ID NO: 2090 |
| TAGCAACC | GCCTCCCTCGCGCCATCAGTAGCAACCCATGCT |
| SEQ ID NO: 2091 | GCCTCCCGTAGGAGT |
| | SEQ ID NO: 2092 |
| TAGCAAGG | GCCTCCCTCGCGCCATCAGTAGCAAGGCATGCT |
| SEQ ID NO: 2093 | GCCTCCCGTAGGAGT |
| | SEQ ID NO: 2094 |
| TAGCATCG | GCCTCCCTCGCGCCATCAGTAGCATCGCATGCT |
| SEQ ID NO: 2095 | GCCTCCCGTAGGAGT |
| | SEQ ID NO: 2096 |
| TAGCATGC | GCCTCCCTCGCGCCATCAGTAGCATGCCATGCT |
| SEQ ID NO: 2097 | GCCTCCCGTAGGAGT |
| | SEQ ID NO: 2098 |
| TAGCCGAT | GCCTCCCTCGCGCCATCAGTAGCCGATCATGCT |
| SEQ ID NO: 2099 | GCCTCCCGTAGGAGT |
| | SEQ ID NO: 2100 |
| TAGCCGTA | GCCTCCCTCGCGCCATCAGTAGCCGTACATGCT |
| SEQ ID NO: 2101 | GCCTCCCGTAGGAGT |
| | SEQ ID NO: 2102 |
| TAGCGCAT | GCCTCCCTCGCGCCATCAGTAGCGCATCATGCT |
| SEQ ID NO: 2103 | GCCTCCCGTAGGAGT |
| | SEQ ID NO: 2104 |
| TAGCGCTA | GCCTCCCTCGCGCCATCAGTAGCGCTACATGCT |
| SEQ ID NO: 2105 | GCCTCCCGTAGGAGT |
| | SEQ ID NO: 2106 |
| TAGCGGAA | GCCTCCCTCGCGCCATCAGTAGCGGAACATGCT |
| SEQ ID NO: 2107 | GCCTCCCGTAGGAGT |
| | SEQ ID NO: 2108 |
| TAGCGGTT | GCCTCCCTCGCGCCATCAGTAGCGGTTCATGCT |
| SEQ ID NO: 2109 | GCCTCCCGTAGGAGT |
| | SEQ ID NO: 2110 |
| TAGCTACG | GCCTCCCTCGCGCCATCAGTAGCTACGCATGCT |
| SEQ ID NO: 2111 | GCCTCCCGTAGGAGT |
| | SEQ ID NO: 2112 |
| TAGCTAGC | GCCTCCCTCGCGCCATCAGTAGCTAGCCATGCT |
| SEQ ID NO: 2113 | GCCTCCCGTAGGAGT |
| | SEQ ID NO: 2114 |
| TAGCTTCC | GCCTCCCTCGCGCCATCAGTAGCTTCCCATGCT |
| SEQ ID NO: 2115 | GCCTCCCGTAGGAGT |
| | SEQ ID NO: 2116 |
| TAGCTTGG | GCCTCCCTCGCGCCATCAGTAGCTTGGCATGCT |
| SEQ ID NO: 2117 | GCCTCCCGTAGGAGT |
| | SEQ ID NO: 2118 |
| TAGGAACG | GCCTCCCTCGCGCCATCAGTAGGAACGCATGCT |
| SEQ ID NO: 2119 | GCCTCCCGTAGGAGT |
| | SEQ ID NO: 2120 |
| TAGGAAGC | GCCTCCCTCGCGCCATCAGTAGGAAGCCATGCT |
| SEQ ID NO: 2121 | GCCTCCCGTAGGAGT |
| | SEQ ID NO: 2122 |
| TAGGATCC | GCCTCCCTCGCGCCATCAGTAGGATCCCATGCT |
| SEQ ID NO: 2123 | GCCTCCCGTAGGAGT |
| | SEQ ID NO: 2134 |
| TAGGATGG | GCCTCCCTCGCGCCATCAGTAGGATGGCATGCT |
| SEQ ID NO: 2135 | GCCTCCCGTAGGAGT |
| | SEQ ID NO: 2136 |
| TAGGCCAT | GCCTCCCTCGCGCCATCAGTAGGCCATCATGCT |
| SEQ ID NO: 2137 | GCCTCCCGTAGGAGT |
| | SEQ ID NO: 2138 |
| TAGGCCTA | GCCTCCCTCGCGCCATCAGTAGGCCTACATGCT |
| SEQ ID NO: 2139 | GCCTCCCGTAGGAGT |
| | SEQ ID NO: 2140 |
| TAGGCGAA | GCCTCCCTCGCGCCATCAGTAGGCGAACATGCT |
| SEQ ID NO: 2141 | GCCTCCCGTAGGAGT |
| | SEQ ID NO: 2142 |
| TAGGCGTT | GCCTCCCTCGCGCCATCAGTAGGCGTTCATGCT |
| SEQ ID NO: 2143 | GCCTCCCGTAGGAGT |
| | SEQ ID NO: 2144 |
| TAGGTACC | GCCTCCCTCGCGCCATCAGTAGGTACCCATGCT |
| SEQ ID NO: 2145 | GCCTCCCGTAGGAGT |
| | SEQ ID NO: 2146 |
| TAGGTAGG | GCCTCCCTCGCGCCATCAGTAGGTAGGCATGCT |
| SEQ ID NO: 2147 | GCCTCCCGTAGGAGT |
| | SEQ ID NO: 2148 |
| TAGGTTCG | GCCTCCCTCGCGCCATCAGTAGGTTCGCATGCT |
| SEQ ID NO: 2149 | GCCTCCCGTAGGAGT |
| | SEQ ID NO: 2150 |
| TAGGTTGC | GCCTCCCTCGCGCCATCAGTAGGTTGCCATGCT |
| SEQ ID NO: 2151 | GCCTCCCGTAGGAGT |
| | SEQ ID NO: 2152 |
| TATACCGG | GCCTCCCTCGCGCCATCAGTATACCGGCATGCT |
| SEQ ID NO: 2153 | GCCTCCCGTAGGAGT |
| | SEQ ID NO: 2154 |
| TATACGCG | GCCTCCCTCGCGCCATCAGTATACGCGCATGCT |
| SEQ ID NO: 2155 | GCCTCCCGTAGGAGT |
| | SEQ ID NO: 2156 |
| TATACGGC | GCCTCCCTCGCGCCATCAGTATACGGCCATGCT |
| SEQ ID NO: 2157 | GCCTCCCGTAGGAGT |
| | SEQ ID NO: 2158 |
| TATAGCCG | GCCTCCCTCGCGCCATCAGTATAGCCGCATGCT |
| SEQ ID NO: 2159 | GCCTCCCGTAGGAGT |
| | SEQ ID NO: 2160 |
| TATAGCGC | GCCTCCCTCGCGCCATCAGTATAGCGCCATGCT |
| SEQ ID NO: 2161 | GCCTCCCGTAGGAGT |
| | SEQ ID NO: 2162 |
| TATAGGCC | GCCTCCCTCGCGCCATCAGTATAGGCCCATGCT |
| SEQ ID NO: 2163 | GCCTCCCGTAGGAGT |
| | SEQ ID NO: 2164 |
| TATTCCGC | GCCTCCCTCGCGCCATCAGTATTCCGCCATGCT |
| SEQ ID NO: 2165 | GCCTCCCGTAGGAGT |
| | SEQ ID NO: 2166 |
| TATTCGCC | GCCTCCCTCGCGCCATCAGTATTCGCCCATGCT |
| SEQ ID NO: 2167 | GCCTCCCGTAGGAGT |
| | SEQ ID NO: 2168 |
| TATTGCGG | GCCTCCCTCGCGCCATCAGTATTGCGGCATGCT |
| SEQ ID NO: 2169 | GCCTCCCGTAGGAGT |
| | SEQ ID NO: 2170 |
| TATTGGCG | GCCTCCCTCGCGCCATCAGTATTGGCGCATGCT |
| SEQ ID NO: 2171 | GCCTCCCGTAGGAGT |
| | SEQ ID NO: 2172 |
| TCACACAG | GCCTCCCTCGCGCCATCAGTCACACAGCATGCT |
| SEQ ID NO: 2173 | GCCTCCCGTAGGAGT |
| | SEQ ID NO: 2174 |
| TCACACTC | GCCTCCCTCGCGCCATCAGTCACACTCCATGCT |
| SEQ ID NO: 2175 | GCCTCCCGTAGGAGT |
| | SEQ ID NO: 2176 |
| TCACAGAC | GCCTCCCTCGCGCCATCAGTCACAGACCATGCT |
| SEQ ID NO: 2177 | GCCTCCCGTAGGAGT |
| | SEQ ID NO: 2178 |
| TCACAGTG | GCCTCCCTCGCGCCATCAGTCACAGTGCATGCT |
| SEQ ID NO: 2179 | GCCTCCCGTAGGAGT |
| | SEQ ID NO: 2180 |
| TCACCACT | GCCTCCCTCGCGCCATCAGTCACCACTCATGCT |
| SEQ ID NO: 2181 | GCCTCCCGTAGGAGT |
| | SEQ ID NO: 2182 |
| TCACCAGA | GCCTCCCTCGCGCCATCAGTCACCAGACATGCT |
| SEQ ID NO: 2183 | GCCTCCCGTAGGAGT |
| | SEQ ID NO: 2184 |
| TCACCTCA | GCCTCCCTCGCGCCATCAGTCACCTCACATGCT |
| SEQ ID NO: 2185 | GCCTCCCGTAGGAGT |
| | SEQ ID NO: 2186 |
| TCACCTGT | GCCTCCCTCGCGCCATCAGTCACCTGTCATGCT |
| SEQ ID NO: 2187 | GCCTCCCGTAGGAGT |
| | SEQ ID NO: 2188 |
| TCACGACA | GCCTCCCTCGCGCCATCAGTCACGACACATGCT |
| SEQ ID NO: 2189 | GCCTCCCGTAGGAGT |
| | SEQ ID NO: 2190 |
| TCACGAGT | GCCTCCCTCGCGCCATCAGTCACGAGTCATGCT |
| SEQ ID NO: 2191 | GCCTCCCGTAGGAGT |
| | SEQ ID NO: 2192 |
| TCACGTCT | GCCTCCCTCGCGCCATCAGTCACGTCTCATGCT |
| SEQ ID NO: 2193 | GCCTCCCGTAGGAGT |
| | SEQ ID NO: 2194 |
| TCACGTGA | GCCTCCCTCGCGCCATCAGTCACGTGACATGCT |
| SEQ ID NO: 2195 | GCCTCCCGTAGGAGT |
| | SEQ ID NO: 2196 |
| TCACTCAC | GCCTCCCTCGCGCCATCAGTCACTCACCATGCT |
| SEQ ID NO: 2197 | GCCTCCCGTAGGAGT |
| | SEQ ID NO: 2198 |
| TCACTCTG | GCCTCCCTCGCGCCATCAGTCACTCTGCATGCT |
| SEQ ID NO: 2199 | GCCTCCCGTAGGAGT |
| | SEQ ID NO: 2200 |
| TCACTGAG | GCCTCCCTCGCGCCATCAGTCACTGAGCATGCT |
| SEQ ID NO: 2201 | GCCTCCCGTAGGAGT |
| | SEQ ID NO: 2202 |
| TCACTGTC | GCCTCCCTCGCGCCATCAGTCACTGTCCATGCT |
| SEQ ID NO: 2203 | GCCTCCCGTAGGAGT |
| | SEQ ID NO: 2204 |
| TCAGACAC | GCCTCCCTCGCGCCATCAGTCAGACACCATGCT |
| SEQ ID NO: 2205 | GCCTCCCGTAGGAGT |
| | SEQ ID NO: 2206 |
| TCAGACTG | GCCTCCCTCGCGCCATCAGTCAGACTGCATGCT |
| SEQ ID NO: 2207 | GCCTCCCGTAGGAGT |
| | SEQ ID NO: 2208 |
| TCAGAGAG | GCCTCCCTCGCGCCATCAGTCAGAGAGCATGCT |
| SEQ ID NO: 2209 | GCCTCCCGTAGGAGT |
| | SEQ ID NO: 2210 |
| TCAGAGTC | GCCTCCCTCGCGCCATCAGTCAGAGTCCATGCT |
| SEQ ID NO: 2211 | GCCTCCCGTAGGAGT |
| | SEQ ID NO: 2212 |
| TCAGCACA | GCCTCCCTCGCGCCATCAGTCAGCACACATGCT |
| SEQ ID NO: 2213 | GCCTCCCGTAGGAGT |
| | SEQ ID NO: 2214 |
| TCAGCAGT | GCCTCCCTCGCGCCATCAGTCAGCAGTCATGCT |
| SEQ ID NO: 2215 | GCCTCCCGTAGGAGT |
| | SEQ ID NO: 2216 |
| TCAGCTCT | GCCTCCCTCGCGCCATCAGTCAGCTCTCATGCT |
| SEQ ID NO: 2217 | GCCTCCCGTAGGAGT |
| | SEQ ID NO: 2218 |
| TCAGCTGA | GCCTCCCTCGCGCCATCAGTCAGCTGACATGCT |
| SEQ ID NO: 2219 | GCCTCCCGTAGGAGT |
| | SEQ ID NO: 2220 |
| TCAGGACT | GCCTCCCTCGCGCCATCAGTCAGGACTCATGCT |
| SEQ ID NO: 2221 | GCCTCCCGTAGGAGT |
| | SEQ ID NO: 2222 |
| TCAGGAGA | GCCTCCCTCGCGCCATCAGTCAGGAGACATGCT |
| SEQ ID NO: 2223 | GCCTCCCGTAGGAGT |
| | SEQ ID NO: 2224 |
| TCAGGTCA | GCCTCCCTCGCGCCATCAGTCAGGTCACATGCT |
| SEQ ID NO: 2225 | GCCTCCCGTAGGAGT |
| | SEQ ID NO: 2226 |
| TCAGGTGT | GCCTCCCTCGCGCCATCAGTCAGGTGTCATGCT |
| SEQ ID NO: 2227 | GCCTCCCGTAGGAGT |
| | SEQ ID NO: 2228 |
| TCAGTCAG | GCCTCCCTCGCGCCATCAGTCAGTCAGCATGCT |
| SEQ ID NO: 2229 | GCCTCCCGTAGGAGT |
| | SEQ ID NO: 2230 |
| TCAGTCTC | GCCTCCCTCGCGCCATCAGTCAGTCTCCATGCT |
| SEQ ID NO: 2231 | GCCTCCCGTAGGAGT |
| | SEQ ID NO: 2232 |
| TCAGTGAC | GCCTCCCTCGCGCCATCAGTCAGTGACCATGCT |
| SEQ ID NO: 2233 | GCCTCCCGTAGGAGT |
| | SEQ ID NO: 2234 |
| TCAGTGTG | GCCTCCCTCGCGCCATCAGTCAGTGTGCATGCT |
| SEQ ID NO: 2235 | GCCTCCCGTAGGAGT |
| | SEQ ID NO: 2236 |
| TCCAACCT | GCCTCCCTCGCGCCATCAGTCCAACCTCATGCT |
| SEQ ID NO: 2237 | GCCTCCCGTAGGAGT |
| | SEQ ID NO: 2238 |
| TCCAACGA | GCCTCCCTCGCGCCATCAGTCCAACGACATGCT |
| SEQ ID NO: 2239 | GCCTCCCGTAGGAGT |
| | SEQ ID NO: 2240 |
| TCCAAGCA | GCCTCCCTCGCGCCATCAGTCCAAGCACATGCT |
| SEQ ID NO: 2241 | GCCTCCCGTAGGAGT |
| | SEQ ID NO: 2242 |
| TCCAAGGT | GCCTCCCTCGCGCCATCAGTCCAAGGTCATGCT |
| SEQ ID NO: 2243 | GCCTCCCGTAGGAGT |
| | SEQ ID NO: 2244 |
| TCCACAAG | GCCTCCCTCGCGCCATCAGTCCACAAGCATGCT |
| SEQ ID NO: 2245 | GCCTCCCGTAGGAGT |
| | SEQ ID NO: 2246 |
| TCCACATC | GCCTCCCTCGCGCCATCAGTCCACATCCATGCT |
| SEQ ID NO: 2247 | GCCTCCCGTAGGAGT |
| | SEQ ID NO: 2248 |
| TCCACTAC | GCCTCCCTCGCGCCATCAGTCCACTACCATGCT |
| SEQ ID NO: 2249 | GCCTCCCGTAGGAGT |
| | SEQ ID NO: 2250 |
| TCCACTTG | GCCTCCCTCGCGCCATCAGTCCACTTGCATGCT |
| SEQ ID NO: 2251 | GCCTCCCGTAGGAGT |
| | SEQ ID NO: 2252 |
| TCCAGAAC | GCCTCCCTCGCGCCATCAGTCCAGAACCATGCT |
| SEQ ID NO: 2253 | GCCTCCCGTAGGAGT |
| | SEQ ID NO: 2254 |
| TCCAGATG | GCCTCCCTCGCGCCATCAGTCCAGATGCATGCT |
| SEQ ID NO: 2255 | GCCTCCCGTAGGAGT |
| | SEQ ID NO: 2256 |
| TCCAGTAG | GCCTCCCTCGCGCCATCAGTCCAGTAGCATGCT |
| SEQ ID NO: 2257 | GCCTCCCGTAGGAGT |
| | SEQ ID NO: 2258 |
| TCCAGTTC | GCCTCCCTCGCGCCATCAGTCCAGTTCCATGCT |
| SEQ ID NO: 2259 | GCCTCCCGTAGGAGT |
| | SEQ ID NO: 2260 |
| TCCATCCA | GCCTCCCTCGCGCCATCAGTCCATCCACATGCT |
| SEQ ID NO: 2261 | GCCTCCCGTAGGAGT |
| | SEQ ID NO: 2262 |
| TCCATCGT | GCCTCCCTCGCGCCATCAGTCCATCGTCATGCT |
| SEQ ID NO: 2263 | GCCTCCCGTAGGAGT |
| | SEQ ID NO: 2264 |
| TCCATGCT | GCCTCCCTCGCGCCATCAGTCCATGCTCATGCT |
| SEQ ID NO: 2265 | GCCTCCCGTAGGAGT |
| | SEQ ID NO: 2266 |
| TCCATGGA | GCCTCCCTCGCGCCATCAGTCCATGGACATGCT |
| SEQ ID NO: 2267 | GCCTCCCGTAGGAGT |
| | SEQ ID NO: 2268 |
| TCCTACCA | GCCTCCCTCGCGCCATCAGTCCTACCACATGCT |
| SEQ ID NO: 2669 | GCCTCCCGTAGGAGT |
| | SEQ ID NO: 2670 |
| TCCTACGT | GCCTCCCTCGCGCCATCAGTCCTACGTCATGCT |
| SEQ ID NO: 2671 | GCCTCCCGTAGGAGT |
| | SEQ ID NO: 2672 |
| TCCTAGCT | GCCTCCCTCGCGCCATCAGTCCTAGCTCATGCT |
| SEQ ID NO: 2673 | GCCTCCCGTAGGAGT |
| | SEQ ID NO: 2674 |
| TCCTAGGA | GCCTCCCTCGCGCCATCAGTCCTAGGACATGCT |
| SEQ ID NO: 2675 | GCCTCCCGTAGGAGT |
| | SEQ ID NO: 2676 |
| TCCTCAAC | GCCTCCCTCGCGCCATCAGTCCTCAACCATGCT |
| SEQ ID NO: 2677 | GCCTCCCGTAGGAGT |
| | SEQ ID NO: 2678 |
| TCCTCATG | GCCTCCCTCGCGCCATCAGTCCTCATGCATGCT |
| SEQ ID NO: 2679 | GCCTCCCGTAGGAGT |
| | SEQ ID NO: 2680 |
| TCCTCTAG | GCCTCCCTCGCGCCATCAGTCCTCTAGCATGCT |
| SEQ ID NO: 2681 | GCCTCCCGTAGGAGT |
| | SEQ ID NO: 2682 |
| TCCTCTTC | GCCTCCCTCGCGCCATCAGTCCTCTTCCATGCT |
| SEQ ID NO: 2683 | GCCTCCCGTAGGAGT |
| | SEQ ID NO: 2684 |
| TCCTGAAG | GCCTCCCTCGCGCCATCAGTCCTGAAGCATGCT |
| SEQ ID NO: 2685 | GCCTCCCGTAGGAGT |
| | SEQ ID NO: 2686 |
| TCCTGATC | GCCTCCCTCGCGCCATCAGTCCTGATCCATGCT |
| SEQ ID NO: 2687 | GCCTCCCGTAGGAGT |
| | SEQ ID NO: 2688 |
| TCCTGTAC | GCCTCCCTCGCGCCATCAGTCCTGTACCATGCT |
| SEQ ID NO: 2689 | GCCTCCCGTAGGAGT |
| | SEQ ID NO: 2690 |
| TCCTGTTG | GCCTCCCTCGCGCCATCAGTCCTGTTGCATGCT |
| SEQ ID NO: 2691 | GCCTCCCGTAGGAGT |
| | SEQ ID NO: 2692 |
| TCCTTCCT | GCCTCCCTCGCGCCATCAGTCCTTCCTCATGCT |
| SEQ ID NO: 2693 | GCCTCCCGTAGGAGT |
| | SEQ ID NO: 2694 |
| TCCTTCGA | GCCTCCCTCGCGCCATCAGTCCTTCGACATGCT |
| SEQ ID NO: 2695 | GCCTCCCGTAGGAGT |
| | SEQ ID NO: 2696 |
| TCCTTGCA | GCCTCCCTCGCGCCATCAGTCCTTGCACATGCT |
| SEQ ID NO: 2697 | GCCTCCCGTAGGAGT |
| | SEQ ID NO: 2698 |
| TCCTTGGT | GCCTCCCTCGCGCCATCAGTCCTTGGTCATGCT |
| SEQ ID NO: 2699 | GCCTCCCGTAGGAGT |
| | SEQ ID NO: 2700 |
| TCGAACCA | GCCTCCCTCGCGCCATCAGTCGAACCACATGCT |
| SEQ ID NO: 2701 | GCCTCCCGTAGGAGT |
| | SEQ ID NO: 2702 |
| TCGAACGT | GCCTCCCTCGCGCCATCAGTCGAACGTCATGCT |
| SEQ ID NO: 2703 | GCCTCCCGTAGGAGT |
| | SEQ ID NO: 2704 |
| TCGAAGCT | GCCTCCCTCGCGCCATCAGTCGAAGCTCATGCT |
| SEQ ID NO: 2705 | GCCTCCCGTAGGAGT |
| | SEQ ID NO: 2706 |
| TCGAAGGA | GCCTCCCTCGCGCCATCAGTCGAAGGACATGCT |
| SEQ ID NO: 2707 | GCCTCCCGTAGGAGT |
| | SEQ ID NO: 2708 |
| TCGACAAC | GCCTCCCTCGCGCCATCAGTCGACAACCATGCT |
| SEQ ID NO: 2709 | GCCTCCCGTAGGAGT |
| | SEQ ID NO: 2710 |
| TCGACATG | GCCTCCCTCGCGCCATCAGTCGACATGCATGCT |
| SEQ ID NO: 2711 | GCCTCCCGTAGGAGT |
| | SEQ ID NO: 2712 |
| TCGACTAG | GCCTCCCTCGCGCCATCAGTCGACTAGCATGCT |
| SEQ ID NO: 2713 | GCCTCCCGTAGGAGT |
| | SEQ ID NO: 2714 |
| TCGACTTC | GCCTCCCTCGCGCCATCAGTCGACTTCCATGCT |
| SEQ ID NO: 2715 | GCCTCCCGTAGGAGT |
| | SEQ ID NO: 2716 |
| TCGAGAAG | GCCTCCCTCGCGCCATCAGTCGAGAAGCATGCT |
| SEQ ID NO: 2717 | GCCTCCCGTAGGAGT |
| | SEQ ID NO: 2718 |
| TCGAGATC | GCCTCCCTCGCGCCATCAGTCGAGATCCATGCT |
| SEQ ID NO: 2719 | GCCTCCCGTAGGAGT |
| | SEQ ID NO: 2720 |
| TCGAGTAC | GCCTCCCTCGCGCCATCAGTCGAGTACCATGCT |
| SEQ ID NO: 2721 | GCCTCCCGTAGGAGT |
| | SEQ ID NO: 2722 |
| TCGAGTTG | GCCTCCCTCGCGCCATCAGTCGAGTTGCATGCT |
| SEQ ID NO: 2723 | GCCTCCCGTAGGAGT |
| | SEQ ID NO: 2724 |
| TCGATCCT | GCCTCCCTCGCGCCATCAGTCGATCCTCATGCT |
| SEQ ID NO: 2725 | GCCTCCCGTAGGAGT |
| | SEQ ID NO: 2726 |
| TCGATCGA | GCCTCCCTCGCGCCATCAGTCGATCGACATGCT |
| SEQ ID NO: 2727 | GCCTCCCGTAGGAGT |
| | SEQ ID NO: 2728 |
| TCGATGCA | GCCTCCCTCGCGCCATCAGTCGATGCACATGCT |
| SEQ ID NO: 2729 | GCCTCCCGTAGGAGT |
| | SEQ ID NO: 2730 |
| TCGATGGT | GCCTCCCTCGCGCCATCAGTCGATGGTCATGCT |
| SEQ ID NO: 2731 | GCCTCCCGTAGGAGT |
| | SEQ ID NO: 2732 |
| TCGTACCT | GCCTCCCTCGCGCCATCAGTCGTACCTCATGCT |
| SEQ ID NO: 2733 | GCCTCCCGTAGGAGT |
| | SEQ ID NO: 2734 |
| TCGTACGA | GCCTCCCTCGCGCCATCAGTCGTACGACATGCT |
| SEQ ID NO: 2735 | GCCTCCCGTAGGAGT |
| | SEQ ID NO: 2736 |
| TCGTAGCA | GCCTCCCTCGCGCCATCAGTCGTAGCACATGCT |
| SEQ ID NO: 2737 | GCCTCCCGTAGGAGT |
| | SEQ ID NO: 2738 |
| TCGTAGGT | GCCTCCCTCGCGCCATCAGTCGTAGGTCATGCT |
| SEQ ID NO: 2739 | GCCTCCCGTAGGAGT |
| | SEQ ID NO: 2740 |
| TCGTCAAG | GCCTCCCTCGCGCCATCAGTCGTCAAGCATGCT |
| SEQ ID NO: 2741 | GCCTCCCGTAGGAGT |
| | SEQ ID NO: 2742 |
| TCGTCATC | GCCTCCCTCGCGCCATCAGTCGTCATCCATGCT |
| SEQ ID NO: 2743 | GCCTCCCGTAGGAGT |
| | SEQ ID NO: 2744 |
| TCGTCTAC | GCCTCCCTCGCGCCATCAGTCGTCTACCATGCT |
| SEQ ID NO: 2745 | GCCTCCCGTAGGAGT |
| | SEQ ID NO: 2746 |
| TCGTCTTG | GCCTCCCTCGCGCCATCAGTCGTCTTGCATGCT |
| SEQ ID NO: 2747 | GCCTCCCGTAGGAGT |
| | SEQ ID NO: 2748 |
| TCGTGAAC | GCCTCCCTCGCGCCATCAGTCGTGAACCATGCT |
| SEQ ID NO: 2749 | GCCTCCCGTAGGAGT |
| | SEQ ID NO: 2750 |
| TCGTGATG | GCCTCCCTCGCGCCATCAGTCGTGATGCATGCT |
| SEQ ID NO: 2751 | GCCTCCCGTAGGAGT |
| | SEQ ID NO: 2752 |
| TCGTGTAG | GCCTCCCTCGCGCCATCAGTCGTGTAGCATGCT |
| SEQ ID NO: 2753 | GCCTCCCGTAGGAGT |
| | SEQ ID NO: 2754 |
| TCGTGTTC | GCCTCCCTCGCGCCATCAGTCGTGTTCCATGCT |
| SEQ ID NO: 2755 | GCCTCCCGTAGGAGT |
| | SEQ ID NO: 2756 |
| TCGTTCCA | GCCTCCCTCGCGCCATCAGTCGTTCCACATGCT |
| SEQ ID NO: 2757 | GCCTCCCGTAGGAGT |
| | SEQ ID NO: 2758 |
| TCGTTCGT | GCCTCCCTCGCGCCATCAGTCGTTCGTCATGCT |
| SEQ ID NO: 2759 | GCCTCCCGTAGGAGT |
| | SEQ ID NO: 2760 |
| TCGTTGCT | GCCTCCCTCGCGCCATCAGTCGTTGCTCATGCT |
| SEQ ID NO: 2761 | GCCTCCCGTAGGAGT |
| | SEQ ID NO: 2762 |
| TCGTTGGA | GCCTCCCTCGCGCCATCAGTCGTTGGACATGCT |
| SEQ ID NO: 2763 | GCCTCCCGTAGGAGT |
| | SEQ ID NO: 2764 |
| TCTCACAC | GCCTCCCTCGCGCCATCAGTCTCACACCATGCT |
| SEQ ID NO: 2765 | GCCTCCCGTAGGAGT |
| | SEQ ID NO: 2766 |
| TCTCACTG | GCCTCCCTCGCGCCATCAGTCTCACTGCATGCT |
| SEQ ID NO: 2767 | GCCTCCCGTAGGAGT |
| | SEQ ID NO: 2768 |
| TCTCAGAG | GCCTCCCTCGCGCCATCAGTCTCAGAGCATGCT |
| SEQ ID NO: 2769 | GCCTCCCGTAGGAGT |
| | SEQ ID NO: 2770 |
| TCTCAGTC | GCCTCCCTCGCGCCATCAGTCTCAGTCCATGCT |
| SEQ ID NO: 2771 | GCCTCCCGTAGGAGT |
| | SEQ ID NO: 2772 |
| TCTCCACA | GCCTCCCTCGCGCCATCAGTCTCCACACATGCT |
| SEQ ID NO: 2773 | GCCTCCCGTAGGAGT |
| | SEQ ID NO: 2774 |
| TCTCCAGT | GCCTCCCTCGCGCCATCAGTCTCCAGTCATGCT |
| SEQ ID NO: 2775 | GCCTCCCGTAGGAGT |
| | SEQ ID NO: 2776 |
| TCTCCTCT | GCCTCCCTCGCGCCATCAGTCTCCTCTCATGCT |
| SEQ ID NO: 2777 | GCCTCCCGTAGGAGT |
| | SEQ ID NO: 2778 |
| TCTCCTGA | GCCTCCCTCGCGCCATCAGTCTCCTGACATGCT |
| SEQ ID NO: 2779 | GCCTCCCGTAGGAGT |
| | SEQ ID NO: 2780 |
| TCTCGACT | GCCTCCCTCGCGCCATCAGTCTCGACTCATGCT |
| SEQ ID NO: 2781 | GCCTCCCGTAGGAGT |
| | SEQ ID NO: 2782 |
| TCTCGAGA | GCCTCCCTCGCGCCATCAGTCTCGAGACATGCT |
| SEQ ID NO: 2783 | GCCTCCCGTAGGAGT |
| | SEQ ID NO: 2784 |
| TCTCGTCA | GCCTCCCTCGCGCCATCAGTCTCGTCACATGCT |
| SEQ ID NO: 2785 | GCCTCCCGTAGGAGT |
| | SEQ ID NO: 2786 |
| TCTCGTGT | GCCTCCCTCGCGCCATCAGTCTCGTGTCATGCT |
| SEQ ID NO: 2787 | GCCTCCCGTAGGAGT |
| | SEQ ID NO: 2788 |
| TCTCTCAG | GCCTCCCTCGCGCCATCAGTCTCTCAGCATGCT |
| SEQ ID NO: 2789 | GCCTCCCGTAGGAGT |
| | SEQ ID NO: 2790 |
| TCTCTCTC | GCCTCCCTCGCGCCATCAGTCTCTCTCCATGCT |
| SEQ ID NO: 2791 | GCCTCCCGTAGGAGT |
| | SEQ ID NO: 2792 |
| TCTCTGAC | GCCTCCCTCGCGCCATCAGTCTCTGACCATGCT |
| SEQ ID NO: 2793 | GCCTCCCGTAGGAGT |
| | SEQ ID NO: 2794 |
| TCTCTGTG | GCCTCCCTCGCGCCATCAGTCTCTGTGCATGCT |
| SEQ ID NO: 2795 | GCCTCCCGTAGGAGT |
| | SEQ ID NO: 2796 |
| TCTGACAG | GCCTCCCTCGCGCCATCAGTCTGACAGCATGCT |
| SEQ ID NO: 2797 | GCCTCCCGTAGGAGT |
| | SEQ ID NO: 2798 |
| TCTGACTC | GCCTCCCTCGCGCCATCAGTCTGACTCCATGCT |
| SEQ ID NO: 2799 | GCCTCCCGTAGGAGT |
| | SEQ ID NO: 2800 |
| TCTGAGAC | GCCTCCCTCGCGCCATCAGTCTGAGACCATGCT |
| SEQ ID NO: 2801 | GCCTCCCGTAGGAGT |
| | SEQ ID NO: 2802 |
| TCTGAGTG | GCCTCCCTCGCGCCATCAGTCTGAGTGCATGCT |
| SEQ ID NO: 2803 | GCCTCCCGTAGGAGT |
| | SEQ ID NO: 2804 |
| TCTGCACT | GCCTCCCTCGCGCCATCAGTCTGCACTCATGCT |
| SEQ ID NO: 2805 | GCCTCCCGTAGGAGT |
| | SEQ ID NO: 2806 |
| TCTGCAGA | GCCTCCCTCGCGCCATCAGTCTGCAGACATGCT |
| SEQ ID NO: 2807 | GCCTCCCGTAGGAGT |
| | SEQ ID NO: 2808 |
| TCTGCTCA | GCCTCCCTCGCGCCATCAGTCTGCTCACATGCT |
| SEQ ID NO: 2809 | GCCTCCCGTAGGAGT |
| | SEQ ID NO: 2810 |
| TCTGCTGT | GCCTCCCTCGCGCCATCAGTCTGCTGTCATGCT |
| SEQ ID NO: 2811 | GCCTCCCGTAGGAGT |
| | SEQ ID NO: 2812 |
| TCTGGACA | GCCTCCCTCGCGCCATCAGTCTGGACACATGCT |
| SEQ ID NO: 2813 | GCCTCCCGTAGGAGT |
| | SEQ ID NO: 2814 |
| TCTGGAGT | GCCTCCCTCGCGCCATCAGTCTGGAGTCATGCT |
| SEQ ID NO: 2815 | GCCTCCCGTAGGAGT |
| | SEQ ID NO: 2816 |
| TCTGGTCT | GCCTCCCTCGCGCCATCAGTCTGGTCTCATGCT |
| SEQ ID NO: 2817 | GCCTCCCGTAGGAGT |
| | SEQ ID NO: 2818 |
| TCTGGTGA | GCCTCCCTCGCGCCATCAGTCTGGTGACATGCT |
| SEQ ID NO: 2819 | GCCTCCCGTAGGAGT |
| | SEQ ID NO: 2820 |
| TCTGTCAC | GCCTCCCTCGCGCCATCAGTCTGTCACCATGCT |
| SEQ ID NO: 2821 | GCCTCCCGTAGGAGT |
| | SEQ ID NO: 2822 |
| TCTGTCTG | GCCTCCCTCGCGCCATCAGTCTGTCTGCATGCT |
| SEQ ID NO: 2823 | GCCTCCCGTAGGAGT |
| | SEQ ID NO: 2824 |
| TCTGTGAG | GCCTCCCTCGCGCCATCAGTCTGTGAGCATGCT |
| SEQ ID NO: 2825 | GCCTCCCGTAGGAGT |
| | SEQ ID NO: 2826 |
| TCTGTGTC | GCCTCCCTCGCGCCATCAGTCTGTGTCCATGCT |
| SEQ ID NO: 2827 | GCCTCCCGTAGGAGT |
| | SEQ ID NO: 2828 |
| TGACACAC | GCCTCCCTCGCGCCATCAGTGACACACCATGCT |
| SEQ ID NO: 2829 | GCCTCCCGTAGGAGT |
| | SEQ ID NO: 2830 |
| TGACACTG | GCCTCCCTCGCGCCATCAGTGACACTGCATGCT |
| SEQ ID NO: 2831 | GCCTCCCGTAGGAGT |
| | SEQ ID NO: 2832 |
| TGACAGAG | GCCTCCCTCGCGCCATCAGTGACAGAGCATGCT |
| SEQ ID NO: 2833 | GCCTCCCGTAGGAGT |
| | SEQ ID NO: 2834 |
| TGACAGTC | GCCTCCCTCGCGCCATCAGTGACAGTCCATGCT |
| SEQ ID NO: 2835 | GCCTCCCGTAGGAGT |
| | SEQ ID NO: 2836 |
| TGACCACA | GCCTCCCTCGCGCCATCAGTGACCACACATGCT |
| SEQ ID NO: 2837 | GCCTCCCGTAGGAGT |
| | SEQ ID NO: 2838 |
| TGACCAGT | GCCTCCCTCGCGCCATCAGTGACCAGTCATGCT |
| SEQ ID NO: 2839 | GCCTCCCGTAGGAGT |
| | SEQ ID NO: 2840 |
| TGACCTCT | GCCTCCCTCGCGCCATCAGTGACCTCTCATGCT |
| SEQ ID NO: 2841 | GCCTCCCGTAGGAGT |
| | SEQ ID NO: 2842 |
| TGACCTGA | GCCTCCCTCGCGCCATCAGTGACCTGACATGCT |
| SEQ ID NO: 2843 | GCCTCCCGTAGGAGT |
| | SEQ ID NO: 2844 |
| TGACGACT | GCCTCCCTCGCGCCATCAGTGACGACTCATGCT |
| SEQ ID NO: 2845 | GCCTCCCGTAGGAGT |
| | SEQ ID NO: 2846 |
| TGACGAGA | GCCTCCCTCGCGCCATCAGTGACGAGACATGCT |
| SEQ ID NO: 2847 | GCCTCCCGTAGGAGT |
| | SEQ ID NO: 2848 |
| TGACGTCA | GCCTCCCTCGCGCCATCAGTGACGTCACATGCT |
| SEQ ID NO: 2849 | GCCTCCCGTAGGAGT |
| | SEQ ID NO: 2850 |
| TGACGTGT | GCCTCCCTCGCGCCATCAGTGACGTGTCATGCT |
| SEQ ID NO: 2851 | GCCTCCCGTAGGAGT |
| | SEQ ID NO: 2852 |
| TGACTCAG | GCCTCCCTCGCGCCATCAGTGACTCAGCATGCT |
| SEQ ID NO: 2853 | GCCTCCCGTAGGAGT |
| | SEQ ID NO: 2854 |
| TGACTCTC | GCCTCCCTCGCGCCATCAGTGACTCTCCATGCT |
| SEQ ID NO: 2855 | GCCTCCCGTAGGAGT |
| | SEQ ID NO: 2856 |
| TGACTGAC | GCCTCCCTCGCGCCATCAGTGACTGACCATGCT |
| SEQ ID NO: 2857 | GCCTCCCGTAGGAGT |
| | SEQ ID NO: 2858 |
| TGACTGTG | GCCTCCCTCGCGCCATCAGTGACTGTGCATGCT |
| SEQ ID NO: 2859 | GCCTCCCGTAGGAGT |
| | SEQ ID NO: 2860 |
| TGAGACAG | GCCTCCCTCGCGCCATCAGTGAGACAGCATGCT |
| SEQ ID NO: 2861 | GCCTCCCGTAGGAGT |
| | SEQ ID NO: 2862 |
| TGAGACTC | GCCTCCCTCGCGCCATCAGTGAGACTCCATGCT |
| SEQ ID NO: 2863 | GCCTCCCGTAGGAGT |
| | SEQ ID NO: 2864 |
| TGAGAGAC | GCCTCCCTCGCGCCATCAGTGAGAGACCATGCT |
| SEQ ID NO: 2865 | GCCTCCCGTAGGAGT |
| | SEQ ID NO: 2866 |
| TGAGAGTG | GCCTCCCTCGCGCCATCAGTGAGAGTGCATGCT |
| SEQ ID NO: 2867 | GCCTCCCGTAGGAGT |
| | SEQ ID NO: 2868 |
| TGAGCACT | GCCTCCCTCGCGCCATCAGTGAGCACTCATGCT |
| SEQ ID NO: 2869 | GCCTCCCGTAGGAGT |
| | SEQ ID NO: 2870 |
| TGAGCAGA | GCCTCCCTCGCGCCATCAGTGAGCAGACATGCT |
| SEQ ID NO: 2871 | GCCTCCCGTAGGAGT |
| | SEQ ID NO: 2872 |
| TGAGCTCA | GCCTCCCTCGCGCCATCAGTGAGCTCACATGCT |
| SEQ ID NO: 2873 | GCCTCCCGTAGGAGT |
| | SEQ ID NO: 2874 |
| TGAGCTGT | GCCTCCCTCGCGCCATCAGTGAGCTGTCATGCT |
| SEQ ID NO: 2875 | GCCTCCCGTAGGAGT |
| | SEQ ID NO: 2876 |
| TGAGGACA | GCCTCCCTCGCGCCATCAGTGAGGACACATGCT |
| SEQ ID NO: 2877 | GCCTCCCGTAGGAGT |
| | SEQ ID NO: 2878 |
| TGAGGAGT | GCCTCCCTCGCGCCATCAGTGAGGAGTCATGCT |
| SEQ ID NO: 2879 | GCCTCCCGTAGGAGT |
| | SEQ ID NO: 2880 |
| TGAGGTCT | GCCTCCCTCGCGCCATCAGTGAGGTCTCATGCT |
| SEQ ID NO: 2881 | GCCTCCCGTAGGAGT |
| | SEQ ID NO: 2882 |
| TGAGGTGA | GCCTCCCTCGCGCCATCAGTGAGGTGACATGCT |
| SEQ ID NO: 2883 | GCCTCCCGTAGGAGT |
| | SEQ ID NO: 2884 |
| TGAGTCAC | GCCTCCCTCGCGCCATCAGTGAGTCACCATGCT |
| SEQ ID NO: 2885 | GCCTCCCGTAGGAGT |
| | SEQ ID NO: 2886 |
| TGAGTCTG | GCCTCCCTCGCGCCATCAGTGAGTCTGCATGCT |
| SEQ ID NO: 2887 | GCCTCCCGTAGGAGT |
| | SEQ ID NO: 2888 |
| TGAGTGAG | GCCTCCCTCGCGCCATCAGTGAGTGAGCATGCT |
| SEQ ID NO: 2889 | GCCTCCCGTAGGAGT |
| | SEQ ID NO: 2890 |
| TGAGTGTC | GCCTCCCTCGCGCCATCAGTGAGTGTCCATGCT |
| SEQ ID NO: 2891 | GCCTCCCGTAGGAGT |
| | SEQ ID NO: 2892 |
| TGCAACCA | GCCTCCCTCGCGCCATCAGTGCAACCACATGCT |
| SEQ ID NO: 2893 | GCCTCCCGTAGGAGT |
| | SEQ ID NO: 2894 |
| TGCAACGT | GCCTCCCTCGCGCCATCAGTGCAACGTCATGCT |
| SEQ ID NO: 2895 | GCCTCCCGTAGGAGT |
| | SEQ ID NO: 2896 |
| TGCAAGCT | GCCTCCCTCGCGCCATCAGTGCAAGCTCATGCT |
| SEQ ID NO: 2897 | GCCTCCCGTAGGAGT |
| | SEQ ID NO: 2898 |
| TGCAAGGA | GCCTCCCTCGCGCCATCAGTGCAAGGACATGCT |
| SEQ ID NO: 2899 | GCCTCCCGTAGGAGT |
| | SEQ ID NO: 2900 |
| TGCACAAC | GCCTCCCTCGCGCCATCAGTGCACAACCATGCT |
| SEQ ID NO: 2901 | GCCTCCCGTAGGAGT |
| | SEQ ID NO: 2902 |
| TGCACATG | GCCTCCCTCGCGCCATCAGTGCACATGCATGCT |
| SEQ ID NO: 2903 | GCCTCCCGTAGGAGT |
| | SEQ ID NO: 2904 |
| TGCACTAG | GCCTCCCTCGCGCCATCAGTGCACTAGCATGCT |
| SEQ ID NO: 2905 | GCCTCCCGTAGGAGT |
| | SEQ ID NO: 2906 |
| TGCACTTC | GCCTCCCTCGCGCCATCAGTGCACTTCCATGCT |
| SEQ ID NO: 2907 | GCCTCCCGTAGGAGT |
| | SEQ ID NO: 2908 |
| TGCAGAAG | GCCTCCCTCGCGCCATCAGTGCAGAAGCATGCT |
| SEQ ID NO: 2909 | GCCTCCCGTAGGAGT |
| | SEQ ID NO: 2910 |
| TGCAGATC | GCCTCCCTCGCGCCATCAGTGCAGATCCATGCT |
| SEQ ID NO: 2911 | GCCTCCCGTAGGAGT |
| | SEQ ID NO: 2912 |
| TGCAGTAC | GCCTCCCTCGCGCCATCAGTGCAGTACCATGCT |
| SEQ ID NO: 2913 | GCCTCCCGTAGGAGT |
| | SEQ ID NO: 2914 |
| TGCAGTTG | GCCTCCCTCGCGCCATCAGTGCAGTTGCATGCT |
| SEQ ID NO: 2915 | GCCTCCCGTAGGAGT |
| | SEQ ID NO: 2916 |
| TGCATCCT | GCCTCCCTCGCGCCATCAGTGCATCCTCATGCT |
| SEQ ID NO: 2917 | GCCTCCCGTAGGAGT |
| | SEQ ID NO: 2918 |
| TGCATCGA | GCCTCCCTCGCGCCATCAGTGCATCGACATGCT |
| SEQ ID NO: 2919 | GCCTCCCGTAGGAGT |
| | SEQ ID NO: 2920 |
| TGCATGCA | GCCTCCCTCGCGCCATCAGTGCATGCACATGCT |
| SEQ ID NO: 2921 | GCCTCCCGTAGGAGT |
| | SEQ ID NO: 2922 |
| TGCATGGT | GCCTCCCTCGCGCCATCAGTGCATGGTCATGCT |
| SEQ ID NO: 2923 | GCCTCCCGTAGGAGT |
| | SEQ ID NO: 2924 |
| TGCTACCT | GCCTCCCTCGCGCCATCAGTGCTACCTCATGCT |
| SEQ ID NO: 2925 | GCCTCCCGTAGGAGT |
| | SEQ ID NO: 2926 |
| TGCTACGA | GCCTCCCTCGCGCCATCAGTGCTACGACATGCT |
| SEQ ID NO: 2927 | GCCTCCCGTAGGAGT |
| | SEQ ID NO: 2928 |
| TGCTAGCA | GCCTCCCTCGCGCCATCAGTGCTAGCACATGCT |
| SEQ ID NO: 2929 | GCCTCCCGTAGGAGT |
| | SEQ ID NO: 2930 |
| TGCTAGGT | GCCTCCCTCGCGCCATCAGTGCTAGGTCATGCT |
| SEQ ID NO: 2931 | GCCTCCCGTAGGAGT |
| | SEQ ID NO: 2932 |
| TGCTCAAG | GCCTCCCTCGCGCCATCAGTGCTCAAGCATGCT |
| SEQ ID NO: 2933 | GCCTCCCGTAGGAGT |
| | SEQ ID NO: 2934 |
| TGCTCATC | GCCTCCCTCGCGCCATCAGTGCTCATCCATGCT |
| SEQ ID NO: 2935 | GCCTCCCGTAGGAGT |
| | SEQ ID NO: 2936 |
| TGCTCTAC | GCCTCCCTCGCGCCATCAGTGCTCTACCATGCT |
| SEQ ID NO: 2937 | GCCTCCCGTAGGAGT |
| | SEQ ID NO: 2938 |
| TGCTCTTG | GCCTCCCTCGCGCCATCAGTGCTCTTGCATGCT |
| SEQ ID NO: 2939 | GCCTCCCGTAGGAGT |
| | SEQ ID NO: 2940 |
| TGCTGAAC | GCCTCCCTCGCGCCATCAGTGCTGAACCATGCT |
| SEQ ID NO: 2941 | GCCTCCCGTAGGAGT |
| | SEQ ID NO: 2942 |
| TGCTGATG | GCCTCCCTCGCGCCATCAGTGCTGATGCATGCT |
| SEQ ID NO: 2943 | GCCTCCCGTAGGAGT |
| | SEQ ID NO: 2944 |
| TGCTGTAG | GCCTCCCTCGCGCCATCAGTGCTGTAGCATGCT |
| SEQ ID NO: 2945 | GCCTCCCGTAGGAGT |
| | SEQ ID NO: 2946 |
| TGCTGTTC | GCCTCCCTCGCGCCATCAGTGCTGTTCCATGCT |
| SEQ ID NO: 2947 | GCCTCCCGTAGGAGT |
| | SEQ ID NO: 2948 |
| TGCTTCCA | GCCTCCCTCGCGCCATCAGTGCTTCCACATGCT |
| SEQ ID NO: 2949 | GCCTCCCGTAGGAGT |
| | SEQ ID NO: 2950 |
| TGCTTCGT | GCCTCCCTCGCGCCATCAGTGCTTCGTCATGCT |
| SEQ ID NO: 2951 | GCCTCCCGTAGGAGT |
| | SEQ ID NO: 2952 |
| TGCTTGCT | GCCTCCCTCGCGCCATCAGTGCTTGCTCATGCT |
| SEQ ID NO: 2953 | GCCTCCCGTAGGAGT |
| | SEQ ID NO: 2954 |
| TGCTTGGA | GCCTCCCTCGCGCCATCAGTGCTTGGACATGCT |
| SEQ ID NO: 2955 | GCCTCCCGTAGGAGT |
| | SEQ ID NO: 2956 |
| TGGAACCT | GCCTCCCTCGCGCCATCAGTGGAACCTCATGCT |
| SEQ ID NO: 2957 | GCCTCCCGTAGGAGT |
| | SEQ ID NO: 2958 |
| TGGAACGA | GCCTCCCTCGCGCCATCAGTGGAACGACATGCT |
| SEQ ID NO: 2959 | GCCTCCCGTAGGAGT |
| | SEQ ID NO: 2960 |
| TGGAAGCA | GCCTCCCTCGCGCCATCAGTGGAAGCACATGCT |
| SEQ ID NO: 2961 | GCCTCCCGTAGGAGT |
| | SEQ ID NO: 2962 |
| TGGAAGGT | GCCTCCCTCGCGCCATCAGTGGAAGGTCATGCT |
| SEQ ID NO: 2963 | GCCTCCCGTAGGAGT |
| | SEQ ID NO: 2964 |
| TGGACAAG | GCCTCCCTCGCGCCATCAGTGGACAAGCATGCT |
| SEQ ID NO: 2965 | GCCTCCCGTAGGAGT |
| | SEQ ID NO: 2966 |
| TGGACATC | GCCTCCCTCGCGCCATCAGTGGACATCCATGCT |
| SEQ ID NO: 2967 | GCCTCCCGTAGGAGT |
| | SEQ ID NO: 2968 |
| TGGACTAC | GCCTCCCTCGCGCCATCAGTGGACTACCATGCT |
| SEQ ID NO: 2969 | GCCTCCCGTAGGAGT |
| | SEQ ID NO: 2970 |
| TGGACTTG | GCCTCCCTCGCGCCATCAGTGGACTTGCATGCT |
| SEQ ID NO: 2971 | GCCTCCCGTAGGAGT |
| | SEQ ID NO: 2972 |
| TGGAGAAC | GCCTCCCTCGCGCCATCAGTGGAGAACCATGCT |
| SEQ ID NO: 2973 | GCCTCCCGTAGGAGT |
| | SEQ ID NO: 2974 |
| TGGAGATG | GCCTCCCTCGCGCCATCAGTGGAGATGCATGCT |
| SEQ ID NO: 2975 | GCCTCCCGTAGGAGT |
| | SEQ ID NO: 2976 |
| TGGAGTAG | GCCTCCCTCGCGCCATCAGTGGAGTAGCATGCT |
| SEQ ID NO: 2977 | GCCTCCCGTAGGAGT |
| | SEQ ID NO: 2978 |
| TGGAGTTC | GCCTCCCTCGCGCCATCAGTGGAGTTCCATGCT |
| SEQ ID NO: 2979 | GCCTCCCGTAGGAGT |
| | SEQ ID NO: 2980 |
| TGGATCCA | GCCTCCCTCGCGCCATCAGTGGATCCACATGCT |
| SEQ ID NO: 2981 | GCCTCCCGTAGGAGT |
| | SEQ ID NO: 2982 |
| TGGATCGT | GCCTCCCTCGCGCCATCAGTGGATCGTCATGCT |
| SEQ ID NO: 2983 | GCCTCCCGTAGGAGT |
| | SEQ ID NO: 2984 |
| TGGATGCT | GCCTCCCTCGCGCCATCAGTGGATGCTCATGCT |
| SEQ ID NO: 2985 | GCCTCCCGTAGGAGT |
| | SEQ ID NO: 2986 |
| TGGATGGA | GCCTCCCTCGCGCCATCAGTGGATGGACATGCT |
| SEQ ID NO: 2987 | GCCTCCCGTAGGAGT |
| | SEQ ID NO: 2988 |
| TGGTACCA | GCCTCCCTCGCGCCATCAGTGGTACCACATGCT |
| SEQ ID NO: 2989 | GCCTCCCGTAGGAGT |
| | SEQ ID NO: 2990 |
| TGGTACGT | GCCTCCCTCGCGCCATCAGTGGTACGTCATGCT |
| SEQ ID NO: 2991 | GCCTCCCGTAGGAGT |
| | SEQ ID NO: 2992 |
| TGGTAGCT | GCCTCCCTCGCGCCATCAGTGGTAGCTCATGCT |
| SEQ ID NO: 2993 | GCCTCCCGTAGGAGT |
| | SEQ ID NO: 2994 |
| TGGTAGGA | GCCTCCCTCGCGCCATCAGTGGTAGGACATGCT |
| SEQ ID NO: 2995 | GCCTCCCGTAGGAGT |
| | SEQ ID NO: 2996 |
| TGGTCAAC | GCCTCCCTCGCGCCATCAGTGGTCAACCATGCT |
| SEQ ID NO: 2997 | GCCTCCCGTAGGAGT |
| | SEQ ID NO: 2998 |
| TGGTCATG | GCCTCCCTCGCGCCATCAGTGGTCATGCATGCT |
| SEQ ID NO: 2999 | GCCTCCCGTAGGAGT |
| | SEQ ID NO: 3000 |
| TGGTCTAG | GCCTCCCTCGCGCCATCAGTGGTCTAGCATGCT |
| SEQ ID NO: 3001 | GCCTCCCGTAGGAGT |
| | SEQ ID NO: 3002 |
| TGGTCTTC | GCCTCCCTCGCGCCATCAGTGGTCTTCCATGCT |
| SEQ ID NO: 3003 | GCCTCCCGTAGGAGT |
| | SEQ ID NO: 3004 |
| TGGTGAAG | GCCTCCCTCGCGCCATCAGTGGTGAAGCATGCT |
| SEQ ID NO: 3005 | GCCTCCCGTAGGAGT |
| | SEQ ID NO: 3006 |
| TGGTGATC | GCCTCCCTCGCGCCATCAGTGGTGATCCATGCT |
| SEQ ID NO: 3007 | GCCTCCCGTAGGAGT |
| | SEQ ID NO: 3008 |
| TGGTGTAC | GCCTCCCTCGCGCCATCAGTGGTGTACCATGCT |
| SEQ ID NO: 3009 | GCCTCCCGTAGGAGT |
| | SEQ ID NO: 3010 |
| TGGTGTTG | GCCTCCCTCGCGCCATCAGTGGTGTTGCATGCT |
| SEQ ID NO: 3011 | GCCTCCCGTAGGAGT |
| | SEQ ID NO: 3012 |
| TGGTTCCT | GCCTCCCTCGCGCCATCAGTGGTTCCTCATGCT |
| SEQ ID NO: 3013 | GCCTCCCGTAGGAGT |
| | SEQ ID NO: 3014 |
| TGGTTCGA | GCCTCCCTCGCGCCATCAGTGGTTCGACATGCT |
| SEQ ID NO: 3015 | GCCTCCCGTAGGAGT |
| | SEQ ID NO: 3016 |
| TGGTTGCA | GCCTCCCTCGCGCCATCAGTGGTTGCACATGCT |
| SEQ ID NO: 3017 | GCCTCCCGTAGGAGT |
| | SEQ ID NO: 3018 |
| TGGTTGGT | GCCTCCCTCGCGCCATCAGTGGTTGGTCATGCT |
| SEQ ID NO: 3019 | GCCTCCCGTAGGAGT |
| | SEQ ID NO: 3020 |
| TGTCACAG | GCCTCCCTCGCGCCATCAGTGTCACAGCATGCT |
| SEQ ID NO: 3021 | GCCTCCCGTAGGAGT |
| | SEQ ID NO: 3022 |
| TGTCACTC | GCCTCCCTCGCGCCATCAGTGTCACTCCATGCT |
| SEQ ID NO: 3023 | GCCTCCCGTAGGAGT |
| | SEQ ID NO: 3024 |
| TGTCAGAC | GCCTCCCTCGCGCCATCAGTGTCAGACCATGCT |
| SEQ ID NO: 3025 | GCCTCCCGTAGGAGT |
| | SEQ ID NO: 3026 |
| TGTCAGTG | GCCTCCCTCGCGCCATCAGTGTCAGTGCATGCT |
| SEQ ID NO: 3027 | GCCTCCCGTAGGAGT |
| | SEQ ID NO: 3028 |
| TGTCCACT | GCCTCCCTCGCGCCATCAGTGTCCACTCATGCT |
| SEQ ID NO: 3029 | GCCTCCCGTAGGAGT |
| | SEQ ID NO: 3030 |
| TGTCCAGA | GCCTCCCTCGCGCCATCAGTGTCCAGACATGCT |
| SEQ ID NO: 3031 | GCCTCCCGTAGGAGT |
| | SEQ ID NO: 3032 |
| TGTCCTCA | GCCTCCCTCGCGCCATCAGTGTCCTCACATGCT |
| SEQ ID NO: 3033 | GCCTCCCGTAGGAGT |
| | SEQ ID NO: 3034 |
| TGTCCTGT | GCCTCCCTCGCGCCATCAGTGTCCTGTCATGCT |
| SEQ ID NO: 3035 | GCCTCCCGTAGGAGT |
| | SEQ ID NO: 3036 |
| TGTCGACA | GCCTCCCTCGCGCCATCAGTGTCGACACATGCT |
| SEQ ID NO: 3037 | GCCTCCCGTAGGAGT |
| | SEQ ID NO: 3038 |
| TGTCGAGT | GCCTCCCTCGCGCCATCAGTGTCGAGTCATGCT |
| SEQ ID NO: 3039 | GCCTCCCGTAGGAGT |
| | SEQ ID NO: 3040 |
| TGTCGTCT | GCCTCCCTCGCGCCATCAGTGTCGTCTCATGCT |
| SEQ ID NO: 3041 | GCCTCCCGTAGGAGT |
| | SEQ ID NO: 3042 |
| TGTCGTGA | GCCTCCCTCGCGCCATCAGTGTCGTGACATGCT |
| SEQ ID NO: 3043 | GCCTCCCGTAGGAGT |
| | SEQ ID NO: 3044 |
| TGTCTCAC | GCCTCCCTCGCGCCATCAGTGTCTCACCATGCT |
| SEQ ID NO: 3045 | GCCTCCCGTAGGAGT |
| | SEQ ID NO: 3046 |
| TGTCTCTG | GCCTCCCTCGCGCCATCAGTGTCTCTGCATGCT |
| SEQ ID NO: 3047 | GCCTCCCGTAGGAGT |
| | SEQ ID NO: 3048 |
| TGTCTGAG | GCCTCCCTCGCGCCATCAGTGTCTGAGCATGCT |
| SEQ ID NO: 3049 | GCCTCCCGTAGGAGT |
| | SEQ ID NO: 3050 |
| TGTCTGTC | GCCTCCCTCGCGCCATCAGTGTCTGTCCATGCT |
| SEQ ID NO: 3051 | GCCTCCCGTAGGAGT |
| | SEQ ID NO: 3052 |
| TGTGACAC | GCCTCCCTCGCGCCATCAGTGTGACACCATGCT |
| SEQ ID NO: 3053 | GCCTCCCGTAGGAGT |
| | SEQ ID NO: 3054 |
| TGTGACTG | GCCTCCCTCGCGCCATCAGTGTGACTGCATGCT |
| SEQ ID NO: 3055 | GCCTCCCGTAGGAGT |
| | SEQ ID NO: 3056 |
| TGTGAGAG | GCCTCCCTCGCGCCATCAGTGTGAGAGCATGCT |
| SEQ ID NO: 3057 | GCCTCCCGTAGGAGT |
| | SEQ ID NO: 3058 |
| TGTGAGTC | GCCTCCCTCGCGCCATCAGTGTGAGTCCATGCT |
| SEQ ID NO: 3059 | GCCTCCCGTAGGAGT |
| | SEQ ID NO: 3060 |
| TGTGCACA | GCCTCCCTCGCGCCATCAGTGTGCACACATGCT |
| SEQ ID NO: 3061 | GCCTCCCGTAGGAGT |
| | SEQ ID NO: 3062 |
| TGTGCAGT | GCCTCCCTCGCGCCATCAGTGTGCAGTCATGCT |
| SEQ ID NO: 3063 | GCCTCCCGTAGGAGT |
| | SEQ ID NO: 3064 |
| TGTGCTCT | GCCTCCCTCGCGCCATCAGTGTGCTCTCATGCT |
| SEQ ID NO: 3065 | GCCTCCCGTAGGAGT |
| | SEQ ID NO: 3066 |
| TGTGCTGA | GCCTCCCTCGCGCCATCAGTGTGCTGACATGCT |
| SEQ ID NO: 3067 | GCCTCCCGTAGGAGT |
| | SEQ ID NO: 3068 |
| TGTGGACT | GCCTCCCTCGCGCCATCAGTGTGGACTCATGCT |
| SEQ ID NO: 3069 | GCCTCCCGTAGGAGT |
| | SEQ ID NO: 3070 |
| TGTGGAGA | GCCTCCCTCGCGCCATCAGTGTGGAGACATGCT |
| SEQ ID NO: 3071 | GCCTCCCGTAGGAGT |
| | SEQ ID NO: 3072 |
| TGTGGTCA | GCCTCCCTCGCGCCATCAGTGTGGTCACATGCT |
| SEQ ID NO: 3073 | GCCTCCCGTAGGAGT |
| | SEQ ID NO: 3074 |
| TGTGGTGT | GCCTCCCTCGCGCCATCAGTGTGGTGTCATGCT |
| SEQ ID NO: 3075 | GCCTCCCGTAGGAGT |
| | SEQ ID NO: 3076 |
| TGTGTCAG | GCCTCCCTCGCGCCATCAGTGTGTCAGCATGCT |
| SEQ ID NO: 3077 | GCCTCCCGTAGGAGT |
| | SEQ ID NO: 3078 |
| TGTGTCTC | GCCTCCCTCGCGCCATCAGTGTGTCTCCATGCT |
| SEQ ID NO: 3079 | GCCTCCCGTAGGAGT |
| | SEQ ID NO: 3080 |
| TGTGTGAC | GCCTCCCTCGCGCCATCAGTGTGTGACCATGCT |
| SEQ ID NO: 3081 | GCCTCCCGTAGGAGT |
| | SEQ ID NO: 3082 |
| TGTGTGTG | GCCTCCCTCGCGCCATCAGTGTGTGTGCATGCT |
| SEQ ID NO: 3083 | GCCTCCCGTAGGAGT |
| | SEQ ID NO: 3084 |
| TTAACCGG | GCCTCCCTCGCGCCATCAGTTAACCGGCATGCT |
| SEQ ID NO: 3085 | GCCTCCCGTAGGAGT |
| | SEQ ID NO: 3086 |
| TTAACGCG | GCCTCCCTCGCGCCATCAGTTAACGCGCATGCT |
| SEQ ID NO: 3087 | GCCTCCCGTAGGAGT |
| | SEQ ID NO: 3088 |
| TTAACGGC | GCCTCCCTCGCGCCATCAGTTAACGGCCATGCT |
| SEQ ID NO: 3089 | GCCTCCCGTAGGAGT |
| | SEQ ID NO: 3090 |
| TTAAGCCG | GCCTCCCTCGCGCCATCAGTTAAGCCGCATGCT |
| SEQ ID NO: 3091 | GCCTCCCGTAGGAGT |
| | SEQ ID NO: 3092 |
| TTAAGCGC | GCCTCCCTCGCGCCATCAGTTAAGCGCCATGCT |
| SEQ ID NO: 3093 | GCCTCCCGTAGGAGT |
| | SEQ ID NO: 3094 |
| TTAAGGCC | GCCTCCCTCGCGCCATCAGTTAAGGCCCATGCT |
| SEQ ID NO: 3095 | GCCTCCCGTAGGAGT |
| | SEQ ID NO: 3096 |
| TTATCCGC | GCCTCCCTCGCGCCATCAGTTATCCGCCATGCT |
| SEQ ID NO: 3097 | GCCTCCCGTAGGAGT |
| | SEQ ID NO: 3098 |
| TTATCGCC | GCCTCCCTCGCGCCATCAGTTATCGCCCATGCT |
| SEQ ID NO: 3099 | GCCTCCCGTAGGAGT |
| | SEQ ID NO: 3100 |
| TTATGCGG | GCCTCCCTCGCGCCATCAGTTATGCGGCATGCT |
| SEQ ID NO: 3101 | GCCTCCCGTAGGAGT |
| | SEQ ID NO: 3102 |
| TTATGGCG | GCCTCCCTCGCGCCATCAGTTATGGCGCATGCT |
| SEQ ID NO: 3103 | GCCTCCCGTAGGAGT |
| | SEQ ID NO: 3104 |
| TTCCAACC | GCCTCCCTCGCGCCATCAGTTCCAACCCATGCT |
| SEQ ID NO: 3105 | GCCTCCCGTAGGAGT |
| | SEQ ID NO: 3106 |
| TTCCAAGG | GCCTCCCTCGCGCCATCAGTTCCAAGGCATGCT |
| SEQ ID NO: 3107 | GCCTCCCGTAGGAGT |
| | SEQ ID NO: 3108 |
| TTCCATCG | GCCTCCCTCGCGCCATCAGTTCCATCGCATGCT |
| SEQ ID NO: 3109 | GCCTCCCGTAGGAGT |
| | SEQ ID NO: 3110 |
| TTCCATGC | GCCTCCCTCGCGCCATCAGTTCCATGCCATGCT |
| SEQ ID NO: 3111 | GCCTCCCGTAGGAGT |
| | SEQ ID NO: 3112 |
| TTCCGCAT | GCCTCCCTCGCGCCATCAGTTCCGCATCATGCT |
| SEQ ID NO: 3113 | GCCTCCCGTAGGAGT |
| | SEQ ID NO: 3114 |
| TTCCGCTA | GCCTCCCTCGCGCCATCAGTTCCGCTACATGCT |
| SEQ ID NO: 3115 | GCCTCCCGTAGGAGT |
| | SEQ ID NO: 3116 |
| TTCCGGAA | GCCTCCCTCGCGCCATCAGTTCCGGAACATGCT |
| SEQ ID NO: 3117 | GCCTCCCGTAGGAGT |
| | SEQ ID NO: 3118 |
| TTCCGGTT | GCCTCCCTCGCGCCATCAGTTCCGGTTCATGCT |
| SEQ ID NO: 3119 | GCCTCCCGTAGGAGT |
| | SEQ ID NO: 3120 |
| TTCCTACG | GCCTCCCTCGCGCCATCAGTTCCTACGCATGCT |
| SEQ ID NO: 3121 | GCCTCCCGTAGGAGT |
| | SEQ ID NO: 3122 |
| TTCCTAGC | GCCTCCCTCGCGCCATCAGTTCCTAGCCATGCT |
| SEQ ID NO: 3123 | GCCTCCCGTAGGAGT |
| | SEQ ID NO: 3124 |
| TTCCTTCC | GCCTCCCTCGCGCCATCAGTTCCTTCCCATGCT |
| SEQ ID NO: 3125 | GCCTCCCGTAGGAGT |
| | SEQ ID NO: 3126 |
| TTCCTTGG | GCCTCCCTCGCGCCATCAGTTCCTTGGCATGCT |
| SEQ ID NO: 3127 | GCCTCCCGTAGGAGT |
| | SEQ ID NO: 3128 |
| TTCGAACG | GCCTCCCTCGCGCCATCAGTTCGAACGCATGCT |
| SEQ ID NO: 3129 | GCCTCCCGTAGGAGT |
| | SEQ ID NO: 3130 |
| TTCGAAGC | GCCTCCCTCGCGCCATCAGTTCGAAGCCATGCT |
| SEQ ID NO: 3131 | GCCTCCCGTAGGAGT |
| | SEQ ID NO: 3132 |
| TTCGATCC | GCCTCCCTCGCGCCATCAGTTCGATCCCATGCT |
| SEQ ID NO: 3133 | GCCTCCCGTAGGAGT |
| | SEQ ID NO: 3134 |
| TTCGATGG | GCCTCCCTCGCGCCATCAGTTCGATGGCATGCT |
| SEQ ID NO: 3135 | GCCTCCCGTAGGAGT |
| | SEQ ID NO: 3136 |
| TTCGCCAT | GCCTCCCTCGCGCCATCAGTTCGCCATCATGCT |
| SEQ ID NO: 3137 | GCCTCCCGTAGGAGT |
| | SEQ ID NO: 3138 |
| TTCGCCTA | GCCTCCCTCGCGCCATCAGTTCGCCTACATGCT |
| SEQ ID NO: 3139 | GCCTCCCGTAGGAGT |
| | SEQ ID NO: 3140 |
| TTCGCGAA | GCCTCCCTCGCGCCATCAGTTCGCGAACATGCT |
| SEQ ID NO: 3141 | GCCTCCCGTAGGAGT |
| | SEQ ID NO: 3142 |
| TTCGCGTT | GCCTCCCTCGCGCCATCAGTTCGCGTTCATGCT |
| SEQ ID NO: 3143 | GCCTCCCGTAGGAGT |
| | SEQ ID NO: 3144 |
| TTCGGCAA | GCCTCCCTCGCGCCATCAGTTCGGCAACATGCT |
| SEQ ID NO: 3145 | GCCTCCCGTAGGAGT |
| | SEQ ID NO: 3146 |
| TTCGGCTT | GCCTCCCTCGCGCCATCAGTTCGGCTTCATGCT |
| SEQ ID NO: 3147 | GCCTCCCGTAGGAGT |
| | SEQ ID NO: 3148 |
| TTCGTACC | GCCTCCCTCGCGCCATCAGTTCGTACCCATGCT |
| SEQ ID NO: 3149 | GCCTCCCGTAGGAGT |
| | SEQ ID NO: 3140 |
| TTCGTAGG | GCCTCCCTCGCGCCATCAGTTCGTAGGCATGCT |
| SEQ ID NO: 3141 | GCCTCCCGTAGGAGT |
| | SEQ ID NO: 3142 |
| TTCGTTCG | GCCTCCCTCGCGCCATCAGTTCGTTCGCATGCT |
| SEQ ID NO: 3143 | GCCTCCCGTAGGAGT |
| | SEQ ID NO: 3144 |
| TTCGTTGC | GCCTCCCTCGCGCCATCAGTTCGTTGCCATGCT |
| SEQ ID NO: 3145 | GCCTCCCGTAGGAGT |
| | SEQ ID NO: 3146 |
| TTGCAACG | GCCTCCCTCGCGCCATCAGTTGCAACGCATGCT |
| SEQ ID NO: 3147 | GCCTCCCGTAGGAGT |
| | SEQ ID NO: 3148 |
| TTGCAAGC | GCCTCCCTCGCGCCATCAGTTGCAAGCCATGCT |
| SEQ ID NO: 3149 | GCCTCCCGTAGGAGT |
| | SEQ ID NO: 3150 |
| TTGCATCC | GCCTCCCTCGCGCCATCAGTTGCATCCCATGCT |
| SEQ ID NO: 3151 | GCCTCCCGTAGGAGT |
| | SEQ ID NO: 3152 |
| TTGCATGG | GCCTCCCTCGCGCCATCAGTTGCATGGCATGCT |
| SEQ ID NO: 3153 | GCCTCCCGTAGGAGT |
| | SEQ ID NO: 3154 |
| TTGCCGAA | GCCTCCCTCGCGCCATCAGTTGCCGAACATGCT |
| SEQ ID NO: 3155 | GCCTCCCGTAGGAGT |
| | SEQ ID NO: 3156 |
| TTGCCGTT | GCCTCCCTCGCGCCATCAGTTGCCGTTCATGCT |
| SEQ ID NO: 3157 | GCCTCCCGTAGGAGT |
| | SEQ ID NO: 3158 |
| TTGCGCAA | GCCTCCCTCGCGCCATCAGTTGCGCAACATGCT |
| SEQ ID NO: 3159 | GCCTCCCGTAGGAGT |
| | SEQ ID NO: 3160 |
| TTGCGCTT | GCCTCCCTCGCGCCATCAGTTGCGCTTCATGCT |
| SEQ ID NO: 3161 | GCCTCCCGTAGGAGT |
| | SEQ ID NO: 3162 |
| TTGCGGAT | GCCTCCCTCGCGCCATCAGTTGCGGATCATGCT |
| SEQ ID NO: 3163 | GCCTCCCGTAGGAGT |
| | SEQ ID NO: 3164 |
| TTGCGGTA | GCCTCCCTCGCGCCATCAGTTGCGGTACATGCT |
| SEQ ID NO: 3165 | GCCTCCCGTAGGAGT |
| | SEQ ID NO: 3166 |
| TTGCTACC | GCCTCCCTCGCGCCATCAGTTGCTACCCATGCT |
| SEQ ID NO: 3167 | GCCTCCCGTAGGAGT |
| | SEQ ID NO: 3168 |
| TTGCTAGG | GCCTCCCTCGCGCCATCAGTTGCTAGGCATGCT |
| SEQ ID NO: 3169 | GCCTCCCGTAGGAGT |
| | SEQ ID NO: 3170 |
| TTGCTTCG | GCCTCCCTCGCGCCATCAGTTGCTTCGCATGCT |
| SEQ ID NO: 3171 | GCCTCCCGTAGGAGT |
| | SEQ ID NO: 3172 |
| TTGCTTGC | GCCTCCCTCGCGCCATCAGTTGCTTGCCATGCT |
| SEQ ID NO: 3173 | GCCTCCCGTAGGAGT |
| | SEQ ID NO: 3174 |
| TTGGAACC | GCCTCCCTCGCGCCATCAGTTGGAACCCATGCT |
| SEQ ID NO: 3175 | GCCTCCCGTAGGAGT |
| | SEQ ID NO: 3176 |
| TTGGAAGG | GCCTCCCTCGCGCCATCAGTTGGAAGGCATGCT |
| SEQ ID NO: 3177 | GCCTCCCGTAGGAGT |
| | SEQ ID NO: 3178 |
| TTGGATCG | GCCTCCCTCGCGCCATCAGTTGGATCGCATGCT |
| SEQ ID NO: 3179 | GCCTCCCGTAGGAGT |
| | SEQ ID NO: 3180 |
| TTGGATGC | GCCTCCCTCGCGCCATCAGTTGGATGCCATGCT |
| SEQ ID NO: 3181 | GCCTCCCGTAGGAGT |
| | SEQ ID NO: 3182 |
| TTGGCCAA | GCCTCCCTCGCGCCATCAGTTGGCCAACATGCT |
| SEQ ID NO: 3183 | GCCTCCCGTAGGAGT |
| | SEQ ID NO: 3184 |
| TTGGCCTT | GCCTCCCTCGCGCCATCAGTTGGCCTTCATGCT |
| SEQ ID NO: 3185 | GCCTCCCGTAGGAGT |
| | SEQ ID NO: 3186 |
| TTGGCGAT | GCCTCCCTCGCGCCATCAGTTGGCGATCATGCT |
| SEQ ID NO: 3187 | GCCTCCCGTAGGAGT |
| | SEQ ID NO: 3188 |
| TTGGCGTA | GCCTCCCTCGCGCCATCAGTTGGCGTACATGCT |
| SEQ ID NO: 3189 | GCCTCCCGTAGGAGT |
| | SEQ ID NO: 3190 |
| TTGGTACG | GCCTCCCTCGCGCCATCAGTTGGTACGCATGCT |
| SEQ ID NO: 3191 | GCCTCCCGTAGGAGT |
| | SEQ ID NO: 3192 |
| TTGGTAGC | GCCTCCCTCGCGCCATCAGTTGGTAGCCATGCT |
| SEQ ID NO: 3193 | GCCTCCCGTAGGAGT |
| | SEQ ID NO: 3194 |
| TTGGTTCC | GCCTCCCTCGCGCCATCAGTTGGTTCCCATGCT |
| SEQ ID NO: 3195 | GCCTCCCGTAGGAGT |
| | SEQ ID NO: 3196 |
| TTGGTTGG | GCCTCCCTCGCGCCATCAGTTGGTTGGCATGCT |
| SEQ ID NO: 3197 | GCCTCCCGTAGGAGT |
| | SEQ ID NO: 3198 |

In some embodiments, the present invention contemplates a method comprising filtering a set of 8 nucleotide base barcodes, and using the filtered barcodes for optimizing PCR and sequencing performance. In one embodiment, the filtering comprises selecting a barcode comprising a GC content of between approximately 40-60%. In one embodiment, the filtering comprises selecting a barcode lacking consecutive triple repeats of the same base (i.e., for example, AAA, TTT, GGG, CCC). In one embodiment, the filtering comprises selecting a barcode lacking perfect self-complementarity or complementarity between the 8-base barcode and the primer. Decoding was performed using a Python translation of an existing C implementation of Hamming codes. R H Morelos-Zaragoza, The Art of Error-Correcting Coding. (John Wiley & Sons, Hoboken, N.J., 2006); and Example II.

A. Barcode Validation

Utility of some embodiments of the present invention may be illustrated by determining the bacterial composition of 286 environmental samples by PCR amplifying, sequencing, and analyzing 681,688 16S rRNA gene sequences from a single sequencing run of the Genome Sequencer FLX (454 Life Sciences, Branford, Conn.). In one particular embodiment, 286 of the 1544 candidate codewords were used to synthesize barcoded PCR primers to use in PCR reactions amplifying a region (27F-338R) of the 16S rRNA gene that were previously determined to be a suitable region of the 16S rRNA to use for phylogenetic analysis from pyrosequencing reads. Wu et al., “Quantitative multiplexing analysis of PCR-amplified ribosomal RNA genes by hierarchical oligonucleotide primer extension reaction” Nucleic Acids Res. 35(11):e82 (2007).

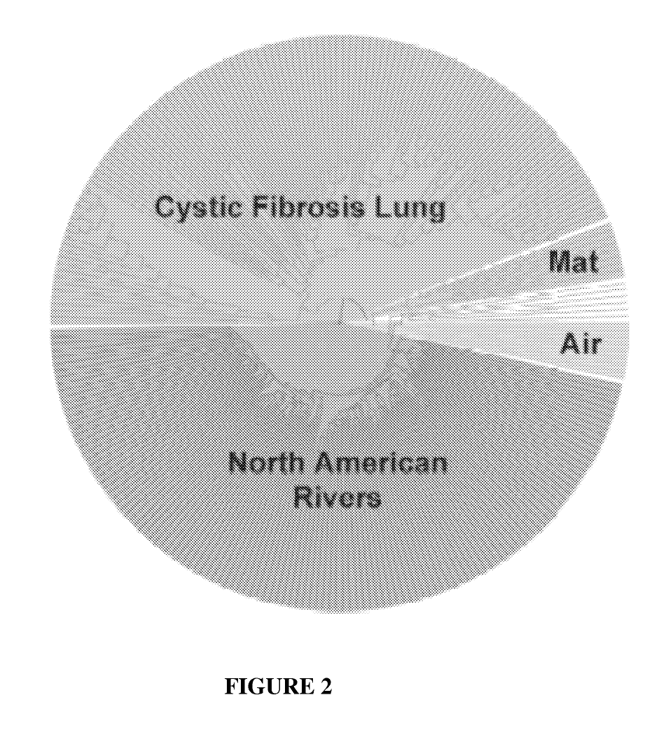

To test these barcodes a set of 1,544 barcodes from the 2,048 possible combinations was chosen based on a nucleotide-encoding scheme that provides the largest number of valid “candidate” barcodes, and then those results were filtered based on optimal PCR and sequencing performance criteria. 286 of the 1,544 candidate barcodes were incorporated into PCR primers that were then used to amplify a region of the bacterial 16S rRNA gene in 286 separate environmental samples. Purified PCR products from each of the 286 samples were then quantified and added to a master DNA pool in equimolar ratios prior to pyrosequencing. Each of the resulting 437,544 sequences was assigned to a sample based on its barcode, aligned based on operational taxonomic units (OTUs) at 96% identity, assembled into a phylogenetic tree and clustered based on similarities in bacterial phylogenetic diversity. The results of this clustering correlated perfectly with sample type—all lung samples clustered together, as did all North American river samples, two African river samples, the microbial mat sample, air samples and hot spring samples. See, FIGS. 2 and 3. These results demonstrate that the tagged barcoding system allows phylogenetic analysis of microbial communities from hundreds of samples in a single sequencing run.

For each sample, the 16S rRNA gene was amplified using the composite forward primer

(SEQ ID NO: 3199)
5′-GCCTTGCCAGCCCGCTCAGTCAGAGTTTGATCCTGGCTCAG-3′:

the underlined sequence is 454 Life Sciences® primer B, and the sequence in italics is the broadly conserved bacterial primer 27F. A two-base linker sequence (‘TC’) that was not observed in >250,000 aligned 16S rRNA sequences was inserted between the 454 Life Sciences® primer B and 27F to help mitigate any effect the composite primer might have on PCR efficiency. The reverse primer was 5′-GCCTCCCTCGCGCCATCAGNNNNNNNN-CATGCTGCCTCCCGTAGGAGT-3′ (SEQ ID NO: 3200): the underlined sequence is 454 Life Sciences® primer A, and the sequence in italics is the broad range bacterial primer 338R. NNNNNNNN designates the unique eight-base barcode used to tag each PCR product, with ‘CA’ inserted as a linker between the barcode and rRNA primer. Total DNA was extracted from samples of a human lung, river water, a Guerrero Negro microbial mat, particles filtered from air, and hot spring water using a modified bead-beating solvent extraction and amplifed by PCR. Dojka et al., Appl Environ Microbiol 64 (10), 3869 (1998).

Briefly, PCR reaction conditions were as follows: 8 μl 2.5X HotMaster PCR Mix (Eppendorf), 0.3 μM each primer, and 10-100 ng template DNA in a total reaction volume of 20 μl. PCR was performed with an Eppendorf Mastercycler: 2 min at 95° C., followed by 30 cycles of 20 s at 95° C. (denaturing), 20 s at 52° C. (annealing) and 60 s at 65° C. (elongation). Four independent PCR reactions were performed for each sample, along with a no template (water) negative control. For each of 286 samples, the four replicate PCR reactions were combined, purified with Ampure magnetic purification beads (Agencourt), quantified with the Quant-iT PicoGreen dsDNA Assay Kit (Invitrogen) and a fluorospectrometer (Nanodrop ND3300), and combined in equimolar ratios to create a master DNA pool with a final concentration of 21.5 ng/μl, which was sent for pyrosequencing with primer A at 454 Life Sciences (Branford, Conn). Margulies et al., Nature 437(7057):376 (2005); Sogin et al., Proc Natl Acad Sci USA 103(32): 12115 (2006). After removal of low-quality sequences and trimming of primer sequences, 437,544 sequences remained, each representing between ˜240-280 bases of 16S rRNA sequence. The quality determination of each sequencing read was based on criteria previously described. Huse et al., Genome Biol 8:R143 (2007). See, Example III.

Each remaining sequence was assigned to a sample based on the barcodes by:
i) picking Operational Taxonomic Units (OTUs) at 96% identity;
ii) aligning one sequence representing each of the 25,351 OTUs with NAST. DeSantis et al., Nucleic Acids Res 34(Web Server Issue), W394 (2006). In comparison, a recent study of 202 globally diverse environments identified only 21,752 OTUs at the 97% level. Lozupone et al., Proc Natl Acad Sci USA 104(27):11436 (2007).
iii) building a “relaxed neighborjoining” tree with clearcut. Sheneman et al., Bioinformatics 22(22):2823 (2006)., and
iv) clustering the samples based on their similarities in bacterial phylogenetic diversity with UniFrac Lozupone et al., BMC Bioinformatics 7:371 (2006); and Lozupone et al., Appl Environ Microbiol 71(12):8228 (2005).

The clustering correlated perfectly with sample types wherein; i) all lung samples clustered together; ii) all North American river samples clustered together; iii) all microbial mat samples clustered together; iv) all air samples clustered together; v) all hot spring samples clustered together; and both African river water samples clustered together. See, FIG. 2.

The clustering was further analyzed to identify distributions of different divisions of bacteria in each of in each of the major sample classes. See, FIG. 3. The samples differ from one another, for example, the cystic fibrosis lung samples are dominated by Firmicutes and gamma-Proteobacteria (mostly Pseudosmona), whereas the Guerrero Negro microbial mat is dominated by Bacteroidetes, Proteobacteria, and Chloroflexi. The results indicate that the pyrosequencing reads provide data comparable to that obtained by traditional approaches.

Nineteen DNA samples were analyzed in triplicate with three independent barcode primers, and in each case the replicate samples clustered together in the UniFrac analysis. This suggests that these barcoded primers amplified equivalently in PCR. 1345 sequences (0.3%) had decoding errors, of which 1241 (92.2%) could be corrected to valid barcodes.

These results directly demonstrated that a tagged barcoding strategy can be used to obtain sequences ranging from approximately the hundreds to approximately the tens of thousands of samples in a single sequencing run. For example, nearly the total number of 16S rRNAs determined to date by Sanger sequencing can be sequenced in a single run using the compositions and methods disclosed herein. Subsequently, a phylogenetic analyses of microbial communities may be perform using the pyrosequencing data.

### Experimental

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

### Example I

### Generation of Error-Correcting Nucleotide Barcodes and Primers

For each sample, a 16S rRNA gene was amplified using a composite forward primer

(SEQ ID NO: 3199)
5′-GCCTTGCCAGCCCGCTCAGTC [symbol] [symbol] [symbol] -3′

wherein the underlined sequence is 454 Life Sciences® primer B, and the bold sequence is the broadly conserved bacterial primer 27F.

Next, a two-base linker sequence (‘TC’) was inserted that was not observed in >250,000 aligned 16S rRNA sequences between the 454 primer B and 27F to help mitigate any effect the composite primer might have on PCR efficiency.

The reverse primer was 5′-GCCTCCCTCGCGCCATCAGNNNNNNNNCA- [symbol] [symbol] [symbol] -3′ (SEQ ID NO: 3200) wherein: i) the underlined sequence is 454 Life Sciences' primer A; ii) the bold sequence is the broad-range bacterial primer 338R; iii) the sequence NNNNNNNN designates the unique eight-base barcode used to tag each PCR product; and iv) ‘CA’ inserted as a linker between the barcode and rRNA primer.

The first 286 barcodes identified in Table 1 were used in the collection of data presented herein.

### Example II

### Barcode Identification Decoding Software

This example presents exemplary software that enables Hamming coding/decoding for pyrosequencing reads and the associated unit tests. This particular program is a command-line application where command-line access depends on the operating system, for example:

Macintosh/Apple OS: Utilities/Terminal:

Microsoft Windows: Start/Run then enter “cmd.exe” in the dialog box:

Linux: Terminal or Shell.

A Python and Numpy packages, available from python.org and numpy.scipy.org, can be downloaded and installed in order to run this software using the Python and the Numpy extension module.

### Example III

### Representative PCR Conditions

PCR reaction conditions were as follows: 8 μl 2.5X HotMaster PCR Mix (Eppendorf), 0.3μM each primer, and 10-100 ng template DNA in a total reaction volume of 20 μl. PCR used an Eppendorf Mastercycler: 120 s at 95° C., followed by 30 cycles of 20 s at 95° C. (denaturing), 20 s at 52° C. (annealing) and 60 s at 65° C. (elongation).

### Example IV

### Processing 454 Reads

Sequences were processed as previously described. Huse et al., Genome Biol 8(7):R143 (2007). In general, the basic steps included, but were not limited to:
1. The read length distribution was examined, and the major peak was identified. Sequences shorter than 237 nt or longer than 283 nt were dropped which were approximately +/−2 standard deviations from the mean of the major peak. This step was performed manually, by inspection of the histogram.
2. Dropped reads with an average quality score less than 25.
3. Dropped reads that contained any ambiguous characters.
4. Split sequence read: first 8 nt provide the barcode (“prefix”). The remainder of the sequence (“suffix”) is used for downstream analyses.
5. Dropped sequences where the suffix does not start with the linker and primer sequence CATGCTGCCTCCCGTAGGAGT.
6. Checked whether the barcode is present in the list of valid barcodes:
   a. If valid, remap to original sample id, assign unique sequence id to the read.
   b. If not, try to correct barcode using the Hamming decoder software in accordance with Example II.
      i. If corrected, remap to original sample id, assign unique sequence id to the read, and record the position and type of the error.
      ii. If not corrected, drop sequence.

### Example V

### OTU Picking Algorithm

OTUs were chosen using the following algorithm:
1. Identify similar sequences using megablast₂. Parameters: E-value 1e-8, minimum coverage 99%, minimum pairwise identity 96%.
2. Find sets of sequences that are connected to one another using BLAST hits at this level.
3. Choose OTUs as follows:
   a. Connected components are candidate OTUs.
   b. The candidate OTU is considered valid if the average density of connections is above 70% (i.e. if 70% of the possible pairwise connections between sequences in the set exist). If the density is lower than this, split up connected component by picking a connected subgraph where the density is above threshold, until no sequences remain in the connected component.
4. A representative sequence was chosen from each OTU by selecting the sequence with the largest number of hits to other sequences in the OTU. Ties were broken by choosing one of the longest sequences within the OTU at random.

### Example VI

### NAST Alignment and Lane Mask

1. The representative set of sequences was aligned using NAST₃ with the following parameters:
   a. Minimum alignment length of 200, and 70% sequence identity.
   b. The template used was the “core_set_aligned.fasta.imputed” (i.e., for example, as posted Aug. 11, 2007 on greengenes.lbl.gov/Download/ Sequence_Data/Fasta_data_files/.
2. The file PH_lanemask, as posted Jul. 18, 2007 greengenes.lbl.gov/Download/Sequence_Data/lanemask_in_{—}1s_and_{—}0s, was used to screen out hypervariable regions of the sequence.

### Example VII

### Tree Building and UniFrac Clustering

1. A relaxed neighbor-joining tree was built using clearcut₄, using the Kimura correction but otherwise with default comparisons.
2. Unweighted UniFrac was run using the resulting tree and the counts of each sequence in each environment. Lozupone et al., Appl Environ Microbiol 71(12): 8228 (2005); and Lozupone et al., BMC Bioinformatics 7:371 (2006).

### Example VIII

### Taxonomy Assignment

Taxonomy was assigned using the best BLAST hit against Greengenes₈, using an E value cutoff of 1e-10, and the Hugenholtz taxonomy. Altschul et al., J Mol Biol 215:403 (1990); and DeSantis et al., Appl Environ Microbiol 72:5069 (2006).

What is claimed is:

1. A pyrosequencing compatible primer comprising a first region containing a unique error-detecting/correcting hamming barcode.

2. The pyrosequencing compatible primer of claim 1, wherein the primer further comprises a second region complementary to a bacterial 16S rRNA gene.

3. A method of assigning sequence data to individual samples from a mixture of samples, comprising:
a) providing:
i) a pyrosequencing compatible primer comprising a first region containing a unique error-detecting/correcting barcode and a second region complementary to a target nucleic acid molecule, and
ii) a target nucleic acid molecule,
b) amplifying said target nucleic acid molecule with said primer,
c) pooling a plurality of said amplification product, and
d) pyrosequencing said pooled amplification products to determine their respective nucleotide sequences.

4. The method of claim 3, wherein said plurality of amplification products are pooled in equimolar ratios.

5. The method of claim 3, wherein said unique error-detecting/correcting barcode is a Hamming code.

6. The method of claim 3, wherein said target nucleic acid molecule comprises a portion of the 16S rRNA gene.

7. The method of claim 3, further comprising identifying amplification products with unique barcode sequence errors.

8. The method of claim 3, further comprising correcting the unique barcode sequence of amplification products containing correctable unique barcode sequence errors.

9. The method of claim 3, further comprising discarding the nucleotide sequence of amplification products containing non-correctable unique barcode sequence errors.

10. The method of claim 3, further comprising step e) aligning the nucleotide sequences of said amplification products to generate a phylogenetic tree.

11. A method comprising:
a) providing:
i) a plurality of samples comprising nucleic acid sequences;
ii) a plurality of primers error correcting or error-detecting sequence tags wherein said primers are at least partially complementary to said nucleic acid sequences:
iii) a parallel sequencing technique capable of simultaneously characterizing said nucleic acid sequences from said plurality of samples;
b) amplifying said plurality of nucleic acid samples using said plurality of primers; and
c) analyzing said sequence tags of said amplified nucleic acids.

12. The method of claim 11, wherein said sequence tag identifies a sample assignment thereby identifying one of said samples from which said nucleic acid was derived.

13. The method of claim 12, wherein said sequence tag identifies the presence of an error in said nucleic acid, thereby establishing a probability that said sample assignment is incorrect.

14. The method of claim 12, wherein said sequence tag identifies the absence of any error in said nucleic acid, thereby establishing a probability that said sample assignment is correct.

15. The method of claim 11, wherein said sequence technique comprises pyrosequencing.
